(19) United States
(12) Patent Application Publication
(10) Pub. No.: US 2018/0055434 A1
(43) Pub. Date: Mar. 1, 2018

(54) SYSTEMS AND METHODS FOR COGNITIVE TESTING

(71) Applicant: Dart NeuroScience, LLC, San Diego, CA (US)

(72) Inventors: Philip Cheung, San Diego, CA (US); Marjorie Rose Principato, San Diego, CA (US); John Austin McNeil, La Jolla, CA (US)

(73) Assignee: Dart NeuroScience, LLC, San Diego, CA (US)

(21) Appl. No.: 15/804,791

(22) Filed: Nov. 6, 2017

Related U.S. Application Data
(63) Continuation-in-part of application No. PCT/US16/31056, filed on May 5, 2016.
Application No. PCT/US16/31056 is a continuation-in-part of application No. PCT/US16/31047, filed on May 5, 2016.
Application No. PCT/US16/31047 is a continuation-in-part of application No. PCT/US16/31051, filed on May 5, 2016.
(60) Provisional application No. 62/157,456, filed on May 5, 2015.
(60) Provisional application No. 62/157,456, filed on May 5, 2015.
(60) Provisional application No. 62/157,456, filed on May 5, 2015.

(51) Int. Cl. A61B 5/16; A01K 29/00; G09B 5/06

(52) U.S. Cl. CPC A61B 5/16; G06F 19/3475; G09B 5/065; A01K 29/005

(57) ABSTRACT

Systems, apparatuses, devices, networks, and methods for testing of animals, and, in particular, cognitive testing. Testing can include a modular hardware controller configured to include at least two modular interfaces, allowing interconnection of one or more child controller circuit boards. The child controller boards may collectively control an environment within the testing chamber, and receive input from or provide output to a testing chamber of an animal testing enclosure. Features are provided to execute testing protocols and collect results, including those using a script-based domain specific language, as well as to adjust a specific test execution using feedback from the testing system to ensure compliance with testing protocols.

### RELATED APPLICATIONS

Any and all priority claims identified in the Application Data Sheet, or any correction thereto, are hereby incorporated by reference under 37 CFR 1.57, the entire contents of which are incorporated herein by reference in their entireties.

### BACKGROUND

### Field

The described technology relates to behavioral testing and training of animals, and more specifically, to systems, apparatuses, devices, networks, and methods for the electronic control of cognitive testing of animals.

### Description of Related Art

Cognition is the process by which an animal acquires, retains, and uses information. It is broadly represented throughout the brain, organized into different domains that govern diverse cognitive functions such as attention, learning, memory, motor skills, language, speech, planning, organizing, sequencing, and abstracting.

Cognitive dysfunction, including the loss of cognitive function, is widespread and increasing in prevalence. Such dysfunction is typically manifested by one or more cognitive deficits, such as memory impairments (impaired ability to acquire new information or to recall previously stored information), aphasia (language/speech disturbance), apraxia (impaired ability to carry out motor activities despite intact motor function), agnosia (failure to recognize or identify objects despite intact sensory function), and disturbances in executive functioning (i.e., planning, organizing, sequencing, abstracting). Cognitive deficits are present in a wide array of neurological conditions and disorders, including age-associated memory impairments, neurodegenerative diseases, and psychiatric disorders, trauma-dependent losses of cognitive function, genetic conditions, mental retardation syndromes, and learning disabilities.

Cognitive testing can be used in numerous applications, such as measuring or assessing a cognitive or motor function, and evaluating the efficacy of a compound or therapeutic in treating a cognitive disorder. Cognitive testing may include training protocols to enhance cognitive function in healthy subjects and improve cognitive function in subjects with cognitive deficits.

Electronic and computer-based approaches to cognitive testing are limited in several ways. Apparatuses and systems implementing such testing are typically based on a centrally-controlled architecture that is subject to output degradation over time, i.e. lack of feedback based control of the environment. A centrally controlled architecture can also be difficult, slow, and expensive to modify in response to desired changes in testing or training devices. Electronic and computer-based approaches can also be unreliable due to poorly controlled variables in the test environment during execution of a test or during a sequence of individual tests. In addition, the software used to carry out cognitive testing is typically based on a static design that impedes—if not precludes—the ability to modify and improve experiments and tests. Thus, there remains a considerable need for methods, systems, devices, networks, and apparatuses that can improve the consistency, reliability, execution, and control of cognitive testing.

### SUMMARY

Systems, apparatuses, devices, networks, and methods disclosed herein each have several aspects, no single one of which is solely responsible for its desirable attributes. Without limiting the scope of this disclosure, for example, as expressed by the claims which follow, its more prominent features will now be discussed briefly. After considering this discussion, and particularly after reading the section entitled “Detailed Description” one will understand how the features being described provide cognitive test execution and control, as well as a modular architecture for an animal testing enclosure.

In some embodiments, cognitive testing of animals may be used to validate the physiological effects of a variety of pharmaceuticals. The cognitive testing may be performed using a testing station that provides a test environment to a subject animal under test. To provide a test environment that is reliable and repeatable, while preventing distractions to the subject animal under test, the testing station may, in some embodiments, enclose the animal being tested within a sealed environment. This enclosure is designed to create a consistent environment, devoid of external stimuli that might introduce variations into the results of the testing process. Within this environment may be one or more devices that can provide controlled stimuli to the animal under test. For example, an electronic display may be provided within the enclosure to facilitate visual stimulus for the animal. One or more input devices may also be included within the enclosure. For example, a touch screen device may receive input from the animal under test. In some cases, input from the animal may be the result of one or more visual representations being displayed on the electronic display. The enclosure may also include a device for introducing a reward, such as a food pellet, to the animal upon the completion of one or more tasks. The enclosure may also include one or more devices for controlling the environment within the enclosure. For example, environmental control of the enclosure may be performed in order to, for example, ensure a constant light level, temperature, or humidity within the enclosure. The environmental control may utilize one or more lights, fans, ducts, or vents to facilitate airflow into and out of the enclosure. Some testing stations may control noise within the enclosure as part of the environmental control. For example, in some testing stations, one or more audio devices, such as speakers, may be utilized to introduce sound, such as white noise, into the enclosure. White noise may be utilized, for example, to reduce an animal under test's perception of sound from outside the enclosure, which could cause distractions to the animal and thus variations in the test results. Disclosed are methods and apparatus for cognitive testing. In specific embodiments, cognitive testing may be used as treatment for memory disorders or to determine the effectiveness of treatments for memory disorders, or to recommend subsequent cognitive exercises. Potential treatments may be tested by administering the treatment to a test subject and observing the subject's performance in various memory games. The test results are compared to those of untreated test subjects and/or the same subject prior to treatment. These games typically require subjects to remember associations between images displayed on a touchscreen. In a game, one or more images may be displayed on the screen. Some subset of these images may be “correct”, and some may be “incorrect”. If the subject chooses the correct image, they may receive a reward, and if they choose the incorrect one, they may not. Over time, the positive reinforcement system can lead to better performance during the experiments, with subjects getting more answers correct. One goal of the testing is for the subject to reach a higher level of accuracy in fewer iterations of game play. Doing so would mean that the treatment under test may have improved the subject's memory.

To implement these experiments, a software system may be provided that manages the layout of the experiment (e.g., which images are displayed, which images are correct, etc.) and a hardware system that displays the images and dispenses rewards. When an experiment is run, the software system sends control messages to the hardware system to tell it how to behave to execute the desired test protocol. The protocol may include setting environmental conditions such as light, noise level, or temperature. The protocol may include displaying one or more images on a display device. The protocol may include receiving a response from the subject such as a touch on a touchscreen or an audio response. The protocol may include triggering a reward or feedback device such as a pellet dispenser or a tone generator. Consistently following the protocol in a provable and reproducible fashion can be important to results obtained from the testing.

Servicing of existing hardware for animal testing apparatuses is extremely expensive and results in significant downtime of a test station. Intermittent test station failures are also common. For example, in some cases, a reward may not be delivered at the appropriate time to the animal under test, potentially introducing an uncontrolled variable into the test results. Lock-ups or freezes in the electronic control of the test stations under use are also relatively common, resulting in additional lost experimentation time, or in some instances, invalidation of a whole series of tests (a study). Furthermore, the architecture of the existing solutions inhibited the integration of new hardware into the test environment, resulting in an overall lack of flexibility. Additionally, the software controlling the above hardware is difficult to control and change.

As a result, previous attempts at enhancing the functionality of existing systems were slow and cumbersome. For example, in one case, an existing system was enhanced to add a feature allowing for repetition of a question when an animal under test (in this case, a monkey) selected an incorrect choice. Due to the lack of modularity in the existing system, six hours of effort was required to reverse engineer the existing system's design and implement the new feature. In view of these issues, the present disclosure provides improved and more accurate control of the testing environment. The methods, apparatus, and devices disclosed herein allow researchers to reproduce near identical testing environments over a variety of tests utilizing a variety of animal subjects. This may be accomplished with minimal effort and error. Furthermore, the system allows additional functionality with less testing downtime. Researchers are then able, for example, to perform tests faster and draw more conclusive results as to a drugs' effectiveness. Accordingly, the system can help researchers decrease the time to market for their drugs. The present disclosure also provides a less expensive solution that performs more reliably than the current commercially available systems. The disclosed improved controller system will enable researchers to gather larger amounts of data that is also more reliable.

The disclosed methods, systems, and devices may make use of a new architecture also disclosed herein that may consist of several modular components. In some embodiments, these components include a central modular controller that includes a motherboard to provide electronic control of the test station. In some aspects, the modular controller may be a modular hardware controller. The modular hardware controller may include, for example, a circuit board (such as a mother board), including an electronic hardware board processor and a bus providing communication between the electronic hardware board processor and an input/output interface for example. The motherboard may include a modular interface connector, including a bus interface, which may connect to one or more modular physical child (or secondary) controller boards that plug into the bus interface on the central modular controller. Each of the physical child controller boards may perform a specific function. For example, a first physical child controller board may provide environmental control of an animal testing enclosure that is part of the test station. Another physical child controller board may control dispensation of a reward to the animal under test. In some aspects, the reward may take the form of a food pellet. Another physical child controller board may control a level of sound within the enclosure. Depending on the design of the cognitive test, any other child controllers can be used, such as ones that track the identity or location of an object or subject (e.g., infrared devices, radio-frequency tags, etc.) or that control response levers, joy sticks, force-feedback devices, multiple displays, cameras, and other devices known in the art, such as those that measure physiological parameters, such as eye dilation, brain activity (e.g., an electroencephalogram), blood pressure, and heart rate.

In some aspects, the master controller and child controllers may not utilize a motherboard/daughterboard configuration. Instead, in some embodiments, each of the master controller and child controllers may be implemented on general purpose computers. In some aspects, the hardware for both the master controller and child controllers may be equivalent. In some aspects, the master controller and child controllers may communicate via an external bus, such as a universal serial bus, local area network, wireless area network, 1394 “Firewire” bus, or other communications interface technology.

The modularization of the architecture proposed herein greatly enhances the flexibility of integration when compared to existing solutions. With the new architecture, as new technologies become available for use in an animal cognitive testing environment, a physical child controller board to control the new technology could be quickly developed and integrated with the controller motherboard. Such an enhancement may not require any changes to the motherboard nor any changes to any of the preexisting physical child controller boards.

The flexibility of the animal testing system is enhanced by designing the controller to be programmable. This programmability may enable not only the controller itself to be controlled, but also enable one or more of the physical child controller boards connected to the controller to be controlled via a programmatic interface.

To solve this problem, make the control software easier to modify, and to provide greater flexibility, a domain specific language (DSL) was developed to control the animal testing system discussed herein. The DSL was designed by a behaviorist for a behaviorist. In certain embodiments, the domain specific language includes built in knowledge of a concurrent discrimination flow. In certain embodiments, the domain specific language includes native support for experimental stages, called intervals. In certain embodiments, the language also supports action primitives, which are operations performed within a particular type of interval. The DSL may also include native support for transitions between different intervals. The DSL can be applied to any cognitive test.

After implementation of the domain specific language, the feature discussed above that provides for repetition of incorrectly answered questions was added to the new system. The time to implement this solution was reduced from the six hours discussed above for the legacy system to fifteen (15) minutes in the new programmatically controlled and modular system disclosed herein.

In certain embodiments, the system includes advanced monitoring of the subjects that are being tested. For example, one or more video recording devices may be configured to record all of some of the cognitive test. In certain embodiments, the systems may be configured to automatically detect and/or identify portions of a recorded video that may be of more interest to investigators than other portions. That is to say the video recording may be indexed along with other recorded events. For example, the system may be configured to detect relatively long periods of inactivity and select the corresponding video recording for this time period. Thus, portions of a recording can be more efficiently investigated by a test administrator. In other embodiments, additional features such as eye-tracking functionality may be added and similarly linked with other data that is collected by the system.

In certain embodiments, the system can further include an indicator in electronic communication with a primary controller to measure an output of the testing unit associated with the one or more parameters. The operation setting of the testing unit can be further based, at least in part, on the results obtained from the one or more indicators or sensors, thereby providing feedback control of the testing unit. This feedback control can be provided by manipulation of the one or more operating parameters sent by the primary controller and/or the feedback control can be provided by manipulation of the operation setting established by the secondary controller.

In certain embodiments, the primary controller can be adapted such that it is capable of electronic communication with an additional secondary controller to provide the testing device with an additional functionality. This functionality can be implemented to the testing device by adding the additional secondary controller while not requiring replacement of the primary controller. In certain embodiments, the secondary controller includes one or more of a reward dispenser, a tone recorder, a noise controller, and an environment controller. Particularly when the tested animal is a non-human animal, the secondary controller can include a reward dispenser. Other ways of rewarding animals, such as providing positive reinforcement to human animals, can also be provided.

In certain aspects, a system for cognitive testing includes a testing apparatus comprising an enclosed space for housing a subject, a central main controller (main controlling means), at least one child controller electronically coupled to the main controller, the at least one child controller electronically coupled to and configured to control at least one environmental device, and at least one sensor disposed within the testing chamber and electronically coupled to the at least one child controller.

In certain aspects, a system for cognitive testing includes a primary controller configured to receive commands from a central hub, the primary controller including a processor and a non-transitory computer readable memory containing instructions for one or more cognitive tests, one or more secondary controllers configured to control one or more parameters of the cognitive test, wherein the testing command causes the primary controller to send the instructions to the one or more secondary controllers which execute the instructions, and one or more sensors configured to verify the performance of the one or more secondary controllers.

The system may also include a device for the controlled dispensing of rewards which may include a base, a mount securing the base to a reward container, the mount configured to secure the reward container at an angle with respect to the base, a pathway coupled to the reward container, the pathway having an entry configured to receive a reward and an exit for dispensing the reward, a gate disposed within the pathway, the gate configured to allow a reward into the pathway, and a feedback sensor configured to output a signal when a reward enters the pathway. In the embodiments of the disclosure, an animal under test may be a non-human animal such as a non-human primate (e.g., a macaque), a non-human mammal (e.g., a dog, cat, rat, or mouse), a non-human vertebrate, or an invertebrate (e.g., a fruit fly). The system may be dynamically adjusted to perform first cognitive testing for a first animal type using a first sequence of testing commands and to perform second cognitive testing for a second animal type using a second sequence of testing commands. In some embodiments, an animal under test can be a human.

One aspect disclosed is an animal testing system for a non-human primate, The system includes a floor, one or more walls coupled to the floor, a top portion supported by the one or more walls, wherein the one or more walls, the floor, and the top portion define a testing chamber, an opening, the opening being sized and shaped to allow the non-human primate to enter the testing chamber, a door being configured to selectively cover the opening, a grate supported by the one or more walls at a location above the floor, an interface positioned above the grate and comprising: a display device disposed so as to be viewable by the non-human primate, a touch device configured to sense contact by the non-human primate, a speaker configured to selectively emit an auditory signal, a pick-up area disposed so as to be accessible by the non-human primate, a dispense light configured to selectively emit a visual signal, a second speaker to emit a controlled background noise to reduce external distractions, a microphone to measure the level of sound produced, house lights to illuminate the testing chamber, and a light sensor configured to measure a light level within the testing chamber; a reward dispenser supported by the top portion and operably connected to the pick-up area; a main controller circuit board supported by the top portion and comprising a plurality of modular interface connectors; and a plurality of child controller circuit boards operably connected to the plurality of modular interface connectors.

In some aspects, a first of the plurality of child controller circuit boards is configured to send control signals to the reward dispenser in response to receipt of a command from the main controller circuit board over the corresponding modular interface connector, and send a response to the main controller circuit board based on feedback signals received from the reward dispenser. In some of these aspects, a second of the plurality of child controller circuit boards is an environmental controller circuit board, and the environmental controller circuit board is configured to control one or more of house lights and a fan within the testing chamber in response to receipt of a command from the main controller circuit board. In some of these aspects, the system also includes a temperature sensor configured to measure a temperature of the testing chamber, and wherein the environmental controller circuit board is further configured to receive the temperature measurement from the temperature sensor and adjust the temperature within the enclosure via the fan. In some of these aspects, the system also includes a light sensor configured to measure ambient light of the testing chamber, and wherein the environmental controller circuit board is further configured to receive the light measurement from the light sensor and adjust the light-level within the enclosure via the house lights.

In some aspects, a third of the plurality of child controller circuit boards is a tone controller circuit board, and wherein the tone controller circuit board is configured to control the speaker in response to receipt of a command from the main controller circuit board. In some of these aspects, a fourth of the plurality of child controller circuit boards is a noise controller circuit board, and the noise controller circuit board is configured to control a level of white noise within the testing chamber via a speaker and a microphone in response to receipt of a command from the main controller circuit board. In some of these aspects, a fifth of the plurality of child controller circuit boards is a display controller circuit board, and wherein the display controller circuit board is configured to control the display device and the touch device. In some of these aspects, the system also includes a subject camera, wherein a sixth child controller circuit board of the plurality of child controller circuit boards is a video controller circuit board, and wherein the video controller circuit board is configured to control video recording of a subject within the testing chamber via the subject camera.

In some aspects of the system the main controller circuit board is configured to present a challenge to the non-human primate in the form of a visual stimulus on the display device and receive input indicating a response from the non-human primate from the touch device.

In some aspects, the system also includes one or more wheels. The wheels may be attached to the floor of the enclosure. In some aspects, the grate is positioned horizontally within the testing chamber at a distance less than 12 inches from the floor. In some aspects, the system includes one or more reinforcement braces attaching the top portion to at least one of the walls.

In some aspects, the animal testing is limited to human testing. In some aspects, the animal testing is limited to non-human testing. In some aspects, animal testing includes cognitive testing. In some aspects, the cognitive testing is used to measure a cognitive or motor function in a subject. In some aspects, the cognitive testing is used to measure a change in a cognitive or motor function in a subject brought about by heredity, disease, injury, or age. In some aspects, the cognitive testing is used to measure a change in a cognitive or motor function in a subject undergoing therapy or treatment of a neurological disorder. In some aspects, cognitive testing includes a training protocol. In some aspects, the training protocol comprises cognitive training. In some aspects, the training protocol comprises motor training. In some aspects, the training protocol comprises process specific tasks. In some aspects, the training protocol comprises skill based tasks. In some aspects, the training protocol is for use in enhancing a cognitive or motor function. In some aspects, the training protocol is for use in rehabilitating a cognitive or motor deficit associated with a neurological disorder. In some aspects, the cognitive deficit is a deficit in memory formation. In some aspects, the deficit in memory formation is a deficit in long term memory formation. In some aspects, the neurological disorder is a neurotrauma. In some aspects, the neurotrauma is stroke or traumatic brain injury. In some aspects, the training protocol is an augmented training protocol and further comprises administering an augmenting agent in conjunction with training.

In one innovative aspect, a system for cognitive testing is provided. The system includes an output device configured to change state during a cognitive test. The system also includes an instruction receiver configured to receive a test instruction including an instruction type and an instruction parameter. The system includes an interpreter in data communication with the instruction receiver and the output device. The interpreter is configured to generate a control amount indicating a quantity of adjustment to the output device by comparing at least a portion of environmental information for an area in which the cognitive test is performed with the instruction parameter. The interpreter is also configured to generate a control message indicating a change in state of the output device using the test instruction and the control amount. The interpreter is further configured to transmit the control message to the output device.

In some implementations of the system, the environmental information for the area includes one of: light, sound, vibration, temperature, humidity, or output level of the output device. Some implementations of the system include a feedback device to detect the environmental information for the area in which the cognitive test is performed.

Some embodiments of the instruction receiver are configured to receive a list of test instructions including the test instruction, the list of test instructions being specified in a domain specific language and parse the test instruction from the list of test instructions using the domain specific language. In such implementations, the interpreter may be configured to identify a format for the control message for the output device using a cognitive testing activity specified in the domain specific language.

Some implementations of the system include a data store configured to store a control message format for a cognitive testing activity for controlling the output device. In such implementations, the interpreter may be configured to generate the control message by at least identifying the control message format for the output device using the cognitive testing activity indicated by the test instruction. Some systems include a second output device configured to change state during the cognitive test. In such implementations, the data store is further configured to store a second control message format for the cognitive testing activity for controlling the second output device. Interpreters included in these implementations may be configured to generate a second control amount indicating a second quantity of adjustment to the second output device by comparing at least a portion of the environmental information with the instruction parameter and generate a second control message indicating a change in state of the second output device using the test instruction and the second control amount. The second control message is different from the control message. The interpreters may also be configured to transmit the second control message to the second output device.

Some implementations of the system include an interpreter that is further configured to receive a response to the state change and store the response in a data store.

Some systems may include a testing coordination server in data communication with a plurality of instruction receivers including the instruction receiver. The testing coordination server may transmit and receive messages to and from at least one of the plurality of instruction receivers associated with a plurality of sequences of testing instructions. The testing coordination server may receive a study design to determine a sequence of testing instruction to be run for a given test subject. The study design may be implemented by logic comprising a fixed list of activities. The study design may be implemented by logic comprising an algorithm that takes into account prior test results to select a test to be run for the given test subject.

For example, the study design may be implemented by logic expressed in a domain specific language to select a test to be run for the given test subject. The test may be selected to further the understanding of cognitive response of the test subject to given conditions or treatments or to determine the cognitive profile of the given test subject. The test to be run may include an assay to diagnose or identify a change in cognitive function brought about by heredity, disease, injury, or age.

The test to be run may include an assay to monitor or measure a response of the given test subject to therapy such as when a subject is undergoing rehabilitation.

The test to be run may include an assay for drug screening. The drug screening may include a protocol for identifying agents that can enhance long term memory. For example, the test to be run may include a training protocol to improve the cognitive function of the test subject either alone or in conjunction with a pharmaceutical treatment.

For example, the training protocol may include instructions to configure the system to perform cognitive training, motor training, process-specific tasks, or skill-based tasks.

In some implementations, the training protocol may include an augmented cognitive training protocol. The augmented cognitive training protocol may include instructions to configure the system to rehabilitate a cognitive or motor deficit such as a neurotrauma disorder (e.g., a stroke, a traumatic brain injury (TBI), a head trauma, or a head injury). The augmented cognitive training protocol includes instructions to configure the system to enhance a cognitive or motor function.

In some implementations, the output device may be configured to adjust an environmental condition in the area in which the cognitive test is performed. Examples of environmental conditions that may be adjusted by the output device include light, sound, temperature, or humidity. The test instruction may include a set-point for the environmental condition. The interpreter may be configured to periodically receive a detected value for the environmental condition and generate a second command message for the output device, the second command message may include information to adjust the environmental condition to the set-point. The information to adjust the environmental condition may be determined based on a comparison of the set-point and the detected value.

In some implementations, the output device may control presentation of a sensory stimulus such as a visual stimulus, an auditory stimulus, a mechanical stimulus, an olfactory stimulus, or a taste stimulus.

In some implementations, the output device may be configured to transmit a message to the interpreter. Examples of such messages initiated by the output device include an acknowledgement, a notification event, an environmental measurement, status confirmation, or warning. The interpreter may be implemented with an interrupt-driven state machine configured to handle these messages from the output device. After receipt at the interpreter, the interrupt-driven state machine may implement a cognitive test protocol.

The interrupt-driven state machine may be configured to identify a subsequent test instruction to execute based on the message received from the output device.

The cognitive test protocol may be expressed in a domain specific language defining a sequence of testing commands in terms of stimuli sets, intervals, response event tests, and system events. The domain specific language may include instructions to cause lookup of previous test results. Generating the control message may be further based on the previous test results obtained. For example, the difficulty or duration of a test may be adjusted based on the previous test results.

The cognitive testing performed by the system may measure a cognitive or motor function in a subject. In addition or in the alternative, the testing may measure a change in a cognitive or motor function in a subject brought about by heredity, disease, injury, or age.

In addition or in the alternative, the cognitive testing may measure a change in a cognitive or motor function in a subject undergoing therapy or treatment of a neurological disorder.

The cognitive test may include instructions to configure the system to present a training protocol such as motor training, process-specific tasks or skill-based tasks. The training protocol may include instructions to configure the output hardware to one or more states to enhance a cognitive or motor function.

The training protocol may include instructions to configure the output hardware to one or more states to rehabilitate a cognitive or motor deficit associated with a neurological disorder such as memory formation, long-term memory formation, neurotrauma (e.g., stroke or traumatic brain injury).

In some implementations, the output device may include or be implemented as a reward dispenser. In such implementations, the test command identifies a quantity of reward to dispense. The reward may include an edible reward such as a pellet, liquid, or paste. An edible reward can also include candy or other food items. In some implementations, the reward may be an inedible reward such as a toy, a coin, or printed material (e.g., coupon, sticker, picture, etc.). In some implementations, the reward may be experiential (e.g., song, video, etc.).

In another innovative aspect, a method of cognitive testing is provided. The method includes receiving a cognitive test configuration indicating a testing unit for performing a cognitive test. The method includes generating a command to adjust a testing hardware element included in the testing unit using the cognitive test configuration and calibration information indicating a state of the testing hardware element. The method also includes transmitting the command to the testing unit.

Examples of the calibration information include light, sound, vibration, temperature, humidity, or output level of the testing hardware element or the testing unit. The method may include receiving the calibration information from the testing unit indicating the state of a testing hardware element included in the testing unit.

The cognitive test configuration may be specified in a domain specific language. The method, in such implementations, may include identifying a format for the command for the testing hardware element using a cognitive testing activity specified in the domain specific language.

Some implementations of the method may include storing the calibration information for the testing hardware element in a data store. These methods may also include determining if the calibration information deviates from a calibration threshold for the testing hardware element and generating an alert for the testing hardware element. For example, the alert may indicate a possible malfunction for the testing hardware element.

The method may include generating a termination command to end the cognitive test. The termination command may include an indication of the possible malfunction for the testing hardware element.

The method may include identifying a resource included in the cognitive test configuration. The method may also include determining the resource is not accessible by the testing unit and transferring the resource to a location accessible by the testing unit.

Some implementations of the method may include receiving a response to the command and storing the response in a data store.

In a further innovative aspect, an apparatus for cognitive testing is provided. The apparatus includes means for receiving a cognitive test configuration indicating a testing unit for performing a cognitive test. The apparatus further includes means for generating a command to adjust a testing hardware element included in the testing unit using the cognitive test configuration and calibration information indicating a state of the testing hardware element. The apparatus also includes means for transmitting the command to the testing unit.

Examples of the calibration information include: light, sound, vibration, temperature, humidity, or output level of the testing hardware element or the testing unit. Some implementations of the apparatus may include means for receiving the calibration information from the testing unit indicating the state of a testing hardware element included in the testing unit. The means for receiving the calibration information may include one or more sensors configured to detect levels of light, temperature, moisture, vibration, or other output level of the testing hardware element or testing unit.

The means for receiving the cognitive test configuration may receive cognitive test configuration in a domain specific language. In such implementations, the apparatus may include means for identifying a format for the command for the testing hardware element using a cognitive testing activity specified in the domain specific language. The means for identifying the format may include an interpreter.

Some implementations of the apparatus may include means for storing the calibration information for the testing hardware element in a data store and means for determining the calibration information deviates from a calibration threshold for the testing hardware element. The means for storing may include a data store or other memory device. The means for determining the calibration information deviates may include a comparison circuit configured to receive the sensed value and a threshold value and provide an output indicating deviation. In some implementations, the means for determining the calibration information deviates may be included in or co-implemented with the interpreter. Where a deviation is detected, the apparatus may include means for generating an alert for the testing hardware element, the alert indicating a possible malfunction for the testing hardware element.

The apparatus may include means for generating a termination command to end the cognitive test. The termination command may include an indication of the possible malfunction for the testing hardware element. The means for generating the termination command may include the interpreter or a message generator configured to receive the possible malfunction and provide a machine-readable message including at least an indication of the possible malfunction.

The apparatus may include means for identifying a resource included in the cognitive test configuration. The apparatus may include means for determining the resource is not accessible by the testing unit and means for transferring the resource to a location accessible by the testing unit. The means for identifying the resource may include the interpreter. The means for determining the resource is not accessible may include one or more sensors configured to detect presences of the identified resource. The resource may be a physical resource (e.g., computing device, item in the testing area, etc.) or a digital resource (e.g., media file, file system location, etc.). The means for transferring may include actuators, motors, servos and other hardware elements to maneuver a physical resource. Where the resource is a digital resource, the means for transferring may include a file transfer client (e.g., file transfer protocol (FTP) client) or other electronic communication device configured to transfer the resource.

The apparatus may include means for receiving a response to the command and means for storing the response in a data store.

In a further innovative aspect, a system for cognitive testing of an animal is provided. The system includes means for providing a sequence of testing commands to a cognitive testing apparatus. The system includes means for receiving a response from the cognitive testing apparatus, the response associated with at least one testing command included in the sequence of testing commands. The system includes means for adjusting the cognitive testing apparatus to provide feedback regarding the response. In some implementations, the means for providing a sequence of testing commands includes a central hub. In some implementations, the means for receiving a response and adjusting the cognitive testing apparatus includes a main controller and one or more independent child controllers.

In some implementations of the system, the means for providing the sequence of testing commands further includes a meta hub configured to communicate, via a network, with a plurality of central hubs, the plurality of central hubs including the central hub.

The meta hub may reside on an internet server and the central hub, the main controller, and the independent child controllers may run on software downloaded to the cognitive testing apparatus at a predetermined secure testing facility.

In some implementations, the meta hub may reside on an internet server and the central hub, the main controller, and the independent child controllers may run on software downloaded to the cognitive testing apparatus of a subject under test. For example, the cognitive testing apparatus may be a portable electronic communication device such as a smartphone or a table computer.

The meta hub may execute in a first thread on an internet server, and the central hub and the main controller may execute in a second thread on the internet server, and a virtual display controller can provide output to a web browser on a remote computer in data communication with the internet server.

The internet server may be implemented as a server cluster configured to share load for at least 10,000 threads to execute corresponding instances of the central hub and the main controller.

In a further innovative aspect, a system for cognitive testing is provided. The system includes an output device configured to indicate or change state during a cognitive test. The system includes an instruction receiver configured to receive a test instruction including an instruction type and an instruction parameter. The system further includes an interpreter in data communication with the instruction receiver and the output device, the interpreter configured to generate a control message about a state of the output device. In some implementations of the system, the interpreter may be configured to generate a control amount indicating a quantity of adjustment to the output device by comparing at least a portion of environmental information for an area in which the cognitive test is performed with the instruction parameter. In some implementations of the system, the interpreter may be configured to generate a control message indicating a change in state of the output device using the test instruction and the control amount. In some implementations of the system, the interpreter may be configured to transmit the control message to the output device.

In a further innovative aspect, a system for cognitive testing is provided. The system includes an output device configured to indicate or change state during a cognitive test. The system includes an instruction receiver configured to receive a test instruction including an instruction type and an instruction parameter. The system includes an interpreter in data communication with the instruction receiver and the output device, the interpreter configured to adjust a specific test execution using feedback from at least one device included in the system. The system for cognitive testing may include additional features discussed above with reference to other innovative systems for cognitive testing.

In some embodiments, the animal under test may be a non-human animal such as a non-human primate (e.g., a macaque), a non-human mammal (e.g., a dog, cat, rat, or mouse), a non-human vertebrate, or an invertebrate (e.g., a fruit fly). The system may be dynamically adjusted to perform first cognitive testing for a first animal type using a first sequence of testing commands and to perform second cognitive testing for a second animal type using a second sequence of testing commands. In other embodiments, the animal under test may be a human, for example, a human in a clinical trial, a human undergoing cognitive assessment, or a human undergoing a training protocol to enhance a cognitive function or improve a cognitive deficit.

One aspect is a system for cognitive testing of an animal, comprising: a central hub processor configured to provide a testing command for a testing station that is configured to accommodate the animal; a plurality of secondary controllers configured to control the testing station, wherein the testing command is associated with one of the plurality of secondary controllers; and a main controller configured to i) receive the testing command from the central hub processor, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers, wherein the one of the plurality of secondary controllers is configured to control the testing station based at least in part on the operating parameter.

In some embodiments, the above system further comprises a printed circuit board, wherein the main controller is supported by the printed circuit board, wherein the plurality of secondary controllers comprise a logical secondary controller positioned within the printed circuit board and a physical secondary controller positioned outside and electrically connected to the printed circuit board. In some embodiments, the logical secondary controller comprises at least one of the following: a display controller configured to control data interface between the animal and the testing station; and a video controller configured to control video streams to and from the testing station. In some embodiments of the above system, the physical secondary controller comprises at least one of the following: a tone controller configured to control a success or failure tone for the cognitive testing; a noise controller configured to control noise levels in the testing station; a reward dispensing controller configured to control reward dispensing in the testing station; and an environmental controller configured to control a testing environment of the testing station.

In some embodiments of the above system, the operating parameter is configured to control the logical and physical secondary controllers to perform their respective control operations on the testing station. In some embodiments, the main controller and the secondary controller are located in the testing station. In some embodiments, the above system further comprises: a central hub simulator configured to simulate an operation of the central hub processor; and a main controller simulator configured to simulate an operation of the main controller. In some embodiments of the above system, the one of the plurality of secondary controllers is configured to control at least one hardware component of the testing station and/or at least one environmental condition in the testing station based at least in part on the operating parameter. In some embodiments of the above system, the at least one hardware component comprises an input device, an output device, a data processing device and a reward dispensing device of the testing station. In some embodiments of the above system, the at least one environmental condition comprises temperature, humidity, light or sound in the testing station.

In some embodiments of the above system, the testing command comprises computer-readable instructions associated with the one of the plurality of secondary controllers. In some embodiments, the main controller is configured to determine the one of the plurality of secondary controllers based on the computer-readable instructions, and generate the operating parameter for the one of the plurality of secondary controllers to control at least one hardware component of the testing station and/or at least one environmental condition in the testing station. In some embodiments of the above system, the animal is a non-human primate. In some embodiments of the above system, the animal is a human.

Another aspect is a system for cognitive testing of an animal, comprising: a main controller configured to receive a testing command from a central hub processor, wherein the testing command is associated with one of a plurality of secondary controllers configured to control a testing station that accommodates the animal, wherein the main controller is further configured to i) determine the one of the plurality of secondary controllers associated with the received testing command, ii) generate an operating parameter for the one of the plurality of secondary controllers based at least in part on the received testing command and iii) provide the generated operating parameter to the one of the plurality of secondary controllers.

In some embodiments, the main controller comprises: a first interface circuit configured to interface data communication between the central hub processor and the main controller; a second interface circuit configured to interface data communication between the main controller and the secondary controller; and a processor configured to determine the one of the plurality of secondary controllers associated with the received testing command and generate the operating parameter for the one of the plurality of secondary controllers based at least in part on the received testing command. In some embodiments, the above system further comprises a memory storing information indicative of commands received from the central hub processor and associated with the plurality of secondary controllers, wherein the processor is configured to determine the one of the plurality of secondary controllers based at least in part on the information stored in the memory.

In some embodiments of the above system, the second interface circuit comprises a plurality of serial ports to be connected to the plurality of secondary controllers, and wherein the processor is configured to detect the one of the plurality of secondary controllers by scanning the serial ports. In some embodiments, the above system further comprises a printed circuit board, wherein the main controller is supported by the printed circuit board, wherein the at least one secondary controller comprises a logical secondary controller positioned within the printed circuit board and a physical secondary controller positioned outside and electrically connected to the printed circuit board.

Another aspect is a system for cognitive testing of an animal, comprising: a plurality of secondary controllers configured to control a testing station that is configured to accommodate the animal; and a main controller configured to i) receive a testing command from a central hub processor, wherein the testing command is associated with one of the plurality of secondary controllers, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers, wherein the one of the plurality of secondary controllers is configured to control the testing station based at least in part on the operating parameter.

In some embodiments, the system further comprises a printed circuit board, wherein the main controller is supported by the printed circuit board, wherein the plurality of secondary controllers comprise a logical secondary controller positioned within the printed circuit board and a physical secondary controller positioned outside and electrically connected to the printed circuit board. In some embodiments, the logical secondary controller comprises at least one of the following: a display controller configured to control data interface between the animal and the testing station; and a video controller configured to control video streams to and from the testing station, and wherein the physical secondary controller comprises at least one of the following: a tone controller configured to control a success or failure tone for the cognitive testing; a noise controller configured to control noise levels in the testing station; a reward dispensing controller configured to control reward dispensing in the testing station; and an environmental controller configured to control a testing environment of the testing station.

Another aspect is a method of cognitive testing of an animal, comprising: providing a plurality of secondary controllers configured to control a testing station that accommodates the animal; receiving a testing command from a central hub processor, wherein the testing command is associated with one of the plurality of secondary controllers; determining the one of the plurality of secondary controllers associated with the received testing command; generating an operating parameter for the one of the plurality of secondary controllers based at least in part on the received testing command; and providing the generated operating parameter to the one of the plurality of secondary controllers.

In some embodiments of the above method, the determining comprises: determining whether the received testing command relates to a logical secondary controller function or a physical secondary controller function; and determining a corresponding physical controller when the received testing command relates to the physical secondary controller function. In some embodiments, the above method further comprises: second determining, when the received testing command relates to the logical secondary controller function, whether the received testing command relates to a display controller function or a video controller function; recognizing a display controller as the one of the plurality of secondary controllers when the received testing command relates to the display controller function; and recognizing a video controller as the one of the plurality of secondary controllers when the received testing command relates to the video controller function. In some embodiments, the cognitive testing is used to measure a cognitive or motor function of the animal. In the above method, the cognitive testing is used to measure a change in a cognitive or motor function of the animal brought about by heredity, disease, injury, or age. In some embodiments, the cognitive testing is used to measure a change in a cognitive or motor function of the animal undergoing therapy or treatment of a neurological disorder. In some embodiments, the cognitive testing includes a training protocol.

In some embodiments, the training protocol comprises cognitive training. In some embodiments, the training protocol comprises motor training. In some embodiments, the training protocol comprises process-specific tasks. In some embodiments, the training protocol comprises skill-based tasks. In some embodiments, the training protocol is for use in enhancing a cognitive or motor function of the animal. In some embodiments, the training protocol is for use in rehabilitating a cognitive or motor deficit associated with a neurological disorder. In some embodiments, the cognitive deficit is a deficit in memory formation. In some embodiments, the deficit in memory formation is a deficit in long-term memory formation. In the above method, the neurological disorder is a neurotrauma. In some embodiments, the neurotrauma is stroke or traumatic brain injury. In some embodiments, the above method further comprises screening for drugs that increase the efficiency of the training protocol. In some embodiments of the method, the training protocol is an augmented training protocol that further comprises administering an augmenting agent in conjunction with training.

Another aspect is a system for cognitive testing of an animal, comprising: means for receiving a testing command from a central hub processor, wherein the testing command is associated with one of a plurality of secondary controllers configured to control a testing station that accommodates the animal; means for determining the one of the plurality of secondary controllers associated with the received testing command; means for generating an operating parameter for the one of the plurality of secondary controllers based at least in part on the received testing command; and means for providing the generated operating parameter to the one of the plurality of secondary controllers.

Another aspect is one or more processor-readable storage devices having processor-readable code embodied on the processor-readable storage devices, the processor-readable code for programming one or more processors to perform a method of cognitive testing of an animal, the method comprising: providing a plurality of secondary controllers configured to control a testing station that accommodates the animal; receiving a testing command from a central hub processor, wherein the testing command is associated with one of the plurality of secondary controllers; determining the one of the plurality of secondary controllers associated with the received testing command; generating an operating parameter for the one of the plurality of secondary controllers based at least in part on the received testing command; and providing the generated operating parameter to the one of the plurality of secondary controllers.

Another aspect is a system for cognitive testing of an animal, comprising: a central hub processor being in data communication with at least one of a main controller and a plurality of secondary controllers configured to control a testing station that accommodates the animal, wherein the central hub processor is configured to send a testing command to the main controller, wherein the testing command is associated with one of the plurality of secondary controllers and configured to control the main controller to i) determine the one of the plurality of secondary controllers associated with the testing command, ii) generate an operating parameter for the one of the plurality of secondary controllers based at least in part on the testing command and iii) provide the generated operating parameter to the one of the plurality of secondary controllers.

In some embodiments of the above system, the testing command comprises computer-readable instructions associated with the one of the plurality of secondary controllers, and the central hub processor is configured to control the main controller to determine the one of the plurality of secondary controllers based on the computer-readable instructions, and generate the operating parameter for the one of the plurality of secondary controllers to control at least one hardware component of the testing station and/or at least one environmental condition in the testing station.

Another aspect is a system for cognitive testing of a non-human animal subject, comprising: a central hub processor configured to provide a sequence of testing commands; a main controller configured to receive the testing commands from the central hub processor and parse the received testing commands; and one or more independent child controllers configured to execute the testing commands, receive responses to the testing commands from the non-human animal subject, and provide feedback regarding the responses.

In some embodiments of the above system, the central hub processor is located on a separate computer and configured to communicate data with the main controller over a network. In some embodiments of the above system, the central hub processor and the main controller are located on the same computer. In some embodiments of the above system, the one or more independent child controllers include a physical child controller. In some embodiments of the above system, the physical child controller comprises an Arduino microcontroller. In the above system, the one or more independent child controllers include a virtual child controller. In some embodiments of the above system, the virtual child controller is located on the main controller. In some embodiments of the above system, the virtual child controller is located on a web browser. In the above system, the web browser is located on the main controller. In some embodiments of the above system, the web browser is located on a separate computer and configured to communicate data with the main controller over a network.

Another aspect is a computer network for cognitive testing of non-human animal subjects, comprising: a plurality of cognitive testing systems, wherein each cognitive testing system comprises a main controller and a plurality of secondary controllers configured to control a testing station that accommodates a non-human animal subject, wherein the main controller is configured to i) receive a testing command, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers, and wherein the one of the plurality of secondary controllers is configured to control the testing station based at least in part on the operating parameter; and a meta hub processor being in data communication with the plurality of cognitive testing systems and configured to automatically coordinate information regarding multiple test subjects and multiple sequences of testing commands among the plurality of cognitive testing systems.

Another aspect is a system for cognitive testing of an animal, comprising: means for providing a sequence of testing commands to the animal; means for parsing the testing commands to different controllers; means for receiving a response to the sequence of testing commands from the animal; and means for providing feedback regarding the response to the animal.

In some embodiments of the above system, the means for providing a sequence of testing commands comprises a central hub processor, wherein the means for parsing comprises a main controller, and wherein the means for receiving a response and the means for providing feedback comprise one or more independent child controllers.

Any of the features of an aspect is applicable to all aspects identified herein. Moreover, any of the features of an aspect is independently combinable, partly or wholly with other aspects described herein in any way, e.g., one, two, or three or more aspects may be combinable in whole or in part. Further, any of the features of an aspect may be made optional to other aspects. Any aspect of a method can comprise another aspect of a cognitive testing system, a cognitive testing network, or a cognitive testing computer network, and any aspect of a cognitive testing system, a cognitive testing network, or a cognitive testing computer network can be configured to perform a method of another aspect.

### BRIEF DESCRIPTION OF THE DRAWINGS

The above-mentioned aspects, as well as other features, aspects, and advantages of the present technology will now be described in connection with various implementations, with reference to the accompanying drawings. The illustrated implementations, however, are merely examples and are not intended to be limiting. Throughout the drawings, similar symbols typically identify similar components, unless context dictates otherwise. Note that the relative dimensions of the following figures may not be drawn to scale.

FIG. 1A show an exemplary system for cognitive testing according to one exemplary embodiment.

FIG. 1AA is a modular architecture for cognitive testing of animals according to a preferred embodiment of the present disclosure.

FIG. 1B is an example configuration utilizing the modular architecture of FIG. 1AA.

FIG. 1C shows two further example configurations utilizing the modular architecture of FIG. 1AA.

FIG. 1D shows another example configuration utilizing the modular architecture of FIG. 1AA.

FIG. 1E shows another example configuration utilizing the modular architecture of FIG. 1AA.

FIG. 1F shows another example configuration utilizing the modular architecture of FIG. 1AA.

FIG. 1G shows another example configuration utilizing the modular architecture of FIG. 1AA.

FIG. 2A shows examples of a reward dispenser and the child controllers shown in FIG. 1AA.

FIG. 2B shows a first side of an exemplary dedicated printed circuit board for the reward dispenser controller illustrated in FIG. 2A.

FIG. 2C shows a second side of the exemplary dedicated printed circuit board for the environmental controller (e.g. pellet dispenser controller) illustrated in FIG. 2A.

FIG. 3A shows an environment controller 103b that may be coupled to a main controller 102 in one exemplary embodiment.

FIG. 3B shows a circuit schematic of one embodiment of the environment controller 103b for use in the modular architecture illustrated in FIG. 2A.

FIG. 4 shows an example printed circuit board (PCB) layout of one embodiment of an environmental controller of FIG. 3B.

FIG. 5A is an illustration of a reward dispenser supported by a mount.

FIG. 5B shows the mount from FIG. 5A.

FIG. 6 shows one example of a reward dispenser architecture system that may be employed with the reward dispenser of FIGS. 2A and/or 5A.

FIG. 7 is an exemplary front view of a reward dispenser door.

FIG. 8 is an exemplary rear view of a reward dispenser door.

FIG. 9 shows a schematic of a printed circuit board for the reward dispenser controller of FIG. 5A.

FIG. 10 shows an example layout of a printed circuit board for the reward dispenser controller of FIG. 5A.

FIG. 11 is an example method for dispensing a pellet using the reward dispenser architecture of FIG. 6.

FIG. 12A illustrates an example architecture for the noise controller circuit board of FIG. 2A.

FIG. 12B illustrates an exemplary embodiment of the modular architecture disclosed herein.

FIG. 13 shows an example schematic for the noise controller of FIG. 2A.

FIG. 14 shows an example printed circuit board layout for the noise controller of FIG. 2A.

FIG. 15 shows an example architecture for the tone controller circuit board of FIG. 2A.

FIG. 16 shows an example schematic for the tone controller circuit board of FIG. 2A.

FIG. 17A shows an example printed circuit board layout for the tone controller of FIG. 2A.

FIG. 18 is a block diagram of a networked system configuration (e.g. Internet based configuration) including subject computers and a global server that employs aspects of the modular architecture illustrated in FIG. 1AA, configured for electronically controlled animal testing.

FIGS. 19-20 are flowcharts of a method that may be performed using the configuration of FIG. 18.

FIG. 21 is a block diagram of a hardware based system configuration including a main controller computer and a global/lab server for implementing aspects of the modular architecture illustrated in FIG. 1AA.

FIG. 22 is a flowchart of a method that may be performed using the system configuration of FIG. 21.

FIG. 23 is a data flow diagram of a study design and test process.

FIG. 24 is an exemplary timing diagram for a reward dispenser.

FIG. 25 illustrates exemplary positioning for a noise controller speaker, microphone, and sound meter.

FIG. 26 shows a flowchart of an example concurrent discrimination test protocol.

FIG. 27 is a listing illustrating an example protocol configuration for a concurrent discrimination experiment protocol.

FIG. 28 illustrates a process flow diagram of an example method of cognitive testing.

FIG. 29 shows a message flow diagram of protocol command execution.

FIG. 30 shows a message flow diagram of child controller event handling.

FIG. 31 shows a message flow diagram of protocol command execution for a study.

FIG. 32 shows a message flow diagram of dynamic environmental calibration.

FIG. 33 shows a message flow diagram of dynamic environmental error detection.

FIG. 34 shows a user interface diagram for a testing system dashboard.

FIG. 35 shows a user interface diagram for testing unit registration and status.

FIG. 36 shows a user interface diagram for viewing test subject information.

FIG. 37 is a block diagram of a cognitive testing system having a modular architecture according to one embodiment.

FIG. 38 is a block diagram of the main controller of FIG. 37 according to one embodiment.

FIG. 39 illustrates an example look-up table of the memory of FIG. 38.

FIG. 40 is a flowchart for an example cognitive testing operation or procedure performed by the processor of FIG. 38.

FIG. 41 shows an example procedure of the determining state of FIG. 40.

FIG. 42 illustrates a cognitive testing simulation system for simulating the central hub and the main controller according to one embodiment.

FIG. 43 illustrates a cognitive testing simulation system for internally simulating the central hub and the main controller according to another embodiment.

FIG. 44 illustrates an exemplary arrangement for a noise controller speaker, a microphone, and a sound meter.

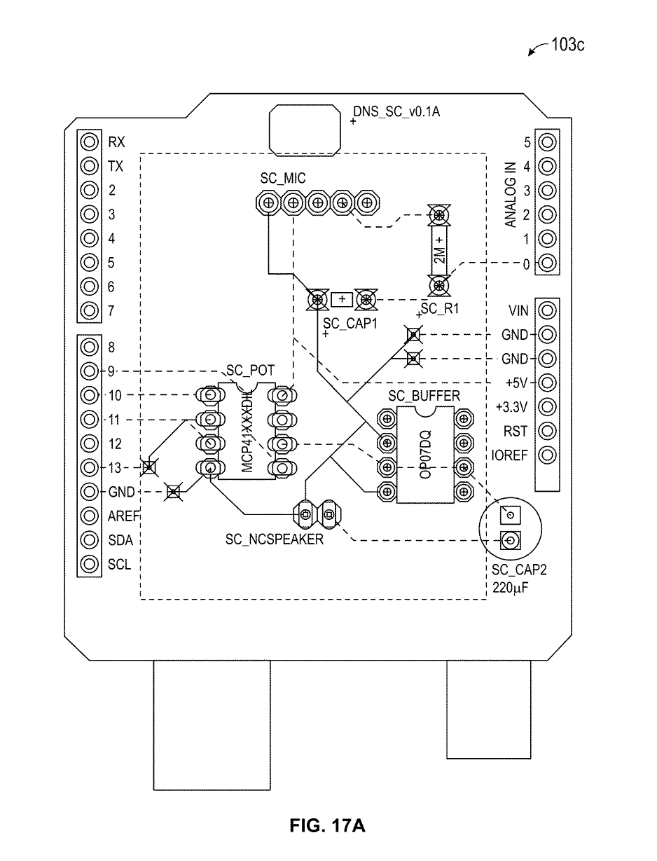

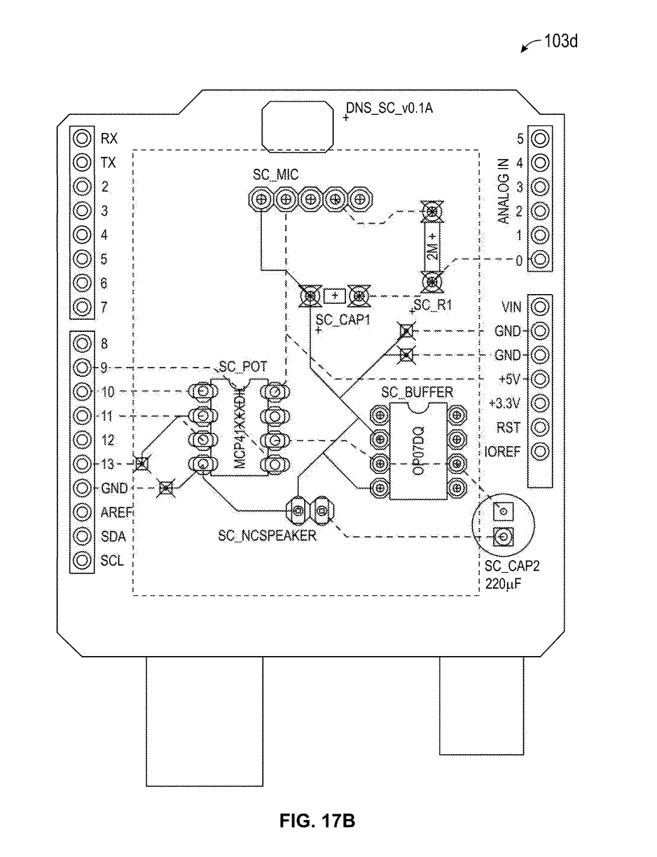

FIG. 17B shows an example printed circuit board layout for a noise controller, such as the noise controller 103d.

### DETAILED DESCRIPTION

Disclosed are methods and systems for cognitive testing.

The architecture disclosed herein can include several modular components. These components include a central controller that includes a mother board to provide electronic control of the test station. The mother board may include a bus interface, which may connect to one or more modular physical child controller boards that plug into the bus interface on the mother board. Each of the physical child controller boards may perform a specific function. For example, a first physical child controller board may provide environmental control of an animal testing enclosure that is part of the test station. Another physical child controller board may control dispensation of a reward to the animal under test. In some embodiments, the reward may take the form of a food pellet. Another physical child controller board may control a level of sound within the enclosure. Depending on the design of the cognitive test, any other child controllers can be used, such as ones that track the identity or location of an object or subject (e.g., infrared devices, radio-frequency tags, etc.) or that control response levers, joy sticks, force-feedback devices, additional displays, cameras, and other devices known in the art, including those that measure physiological parameters, such as eye dilation, brain activity (e.g., EEG), blood pressure, and heart rate.

The modularization of the architecture greatly enhanced the flexibility of integration when compared to existing solutions. With the new architecture, as new technologies become available for use in an animal cognitive testing environment, a physical child controller board to control the new technology could be quickly developed and integrated with the controller mother board. Such an enhancement may not require any significant changes to the mother board nor any changes to any of the preexisting physical child controller boards.

The flexibility of the animal testing system was also greatly enhanced by designing the controller to be programmable. This programmability may enable not only the controller itself to be controlled, but also enable one or more of the physical child controller boards connected to the controller to be controlled via a programmatic interface.

Furthermore, enhancing the functionality of the existing systems was slow and cumbersome. For example, in one case, an existing system was enhanced to add a feature allowing for repetition of a question when an animal under test (in this case, a monkey) selected an incorrect choice. Due to the lack of modularity in the existing system, six hours of effort were required to reverse engineer the existing system's design and implement the new feature.

To solve this problem and provide greater flexibility, a domain specific language (DSL) was developed to control the animal testing system discussed herein. The DSL was designed by a behaviorist for a behaviorist. In certain embodiments, the domain specific language includes built in knowledge of a concurrent discrimination flow. In certain embodiments, the domain specific language includes native support for experimental stages, called intervals. In certain embodiments, the language also supports action primitives, which are operations performed within a particular type of interval. The DSL may also include native support for transitions between different intervals. The DSL can be applied to any cognitive test.

After implementation of the domain specific language, the feature discussed above that provides for repetition of incorrectly answered questions was added to the new system. The time to implement this solution was reduced from the six hours discussed above for the legacy system to about 15 minutes in the new programmatically controlled and modular system disclosed herein.

The disclosed technology relates to electronic control of an animal test station. The electronic control system includes modular components, allowing the system to be easily enhanced and modified without disrupting the overall system design of the electronic control system. Features are described which include use of a script-based domain specific configuration language to represent test protocols. Additional features execute the tests across test systems (e.g., multiple test stations) and collect results. Dynamic test control features adjust a specific test execution using feedback from the testing system, such as a hardware component included in the system, to ensure the test complies with the protocol. The features can help reduce environmental and procedural variability to ensure the cognitive variable(s) under test are isolated and controlled locally to comply with the protocol.

Although the invention has been described with reference to the above example, it will be understood that modifications and variations are encompassed within the spirit and scope of the invention. Accordingly, the invention is limited only by the following claims.

Embodiments will be described with respect to the accompanying drawings. Like reference numerals refer to like elements throughout the detailed description. In this disclosure, the term “substantially” includes the meanings of completely, almost completely or to any significant degree under some applications and in accordance with those skilled in the art. The term “connected” includes an electrical connection.

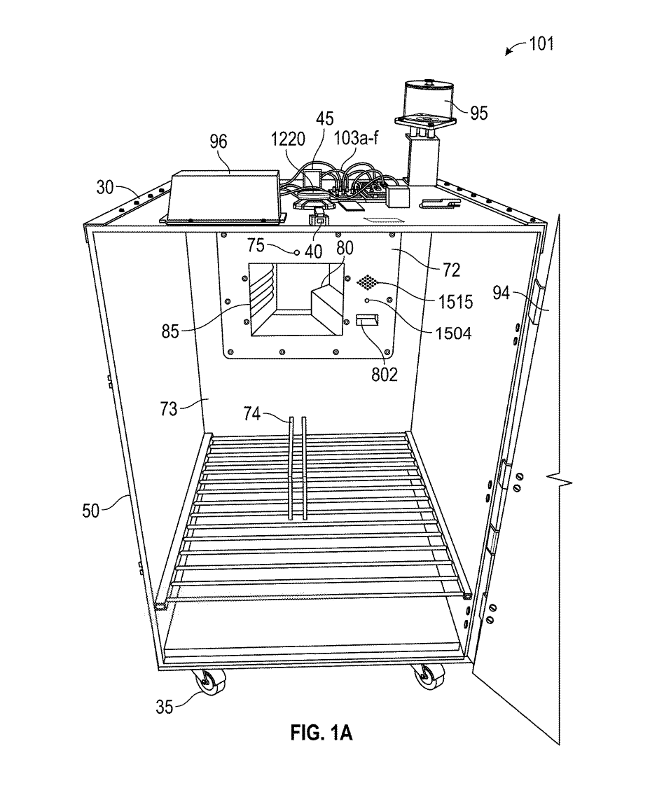

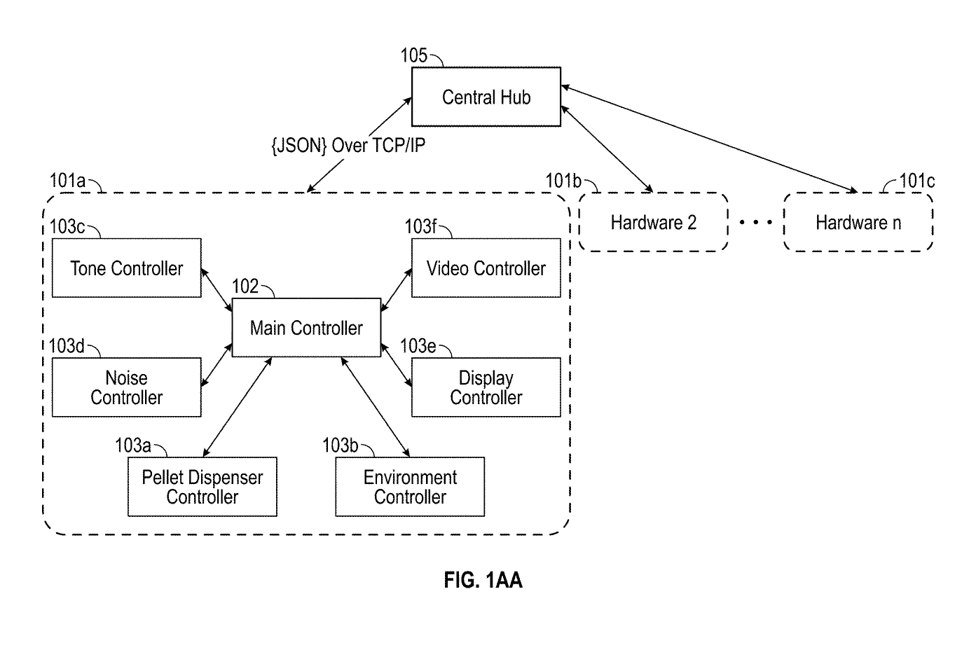

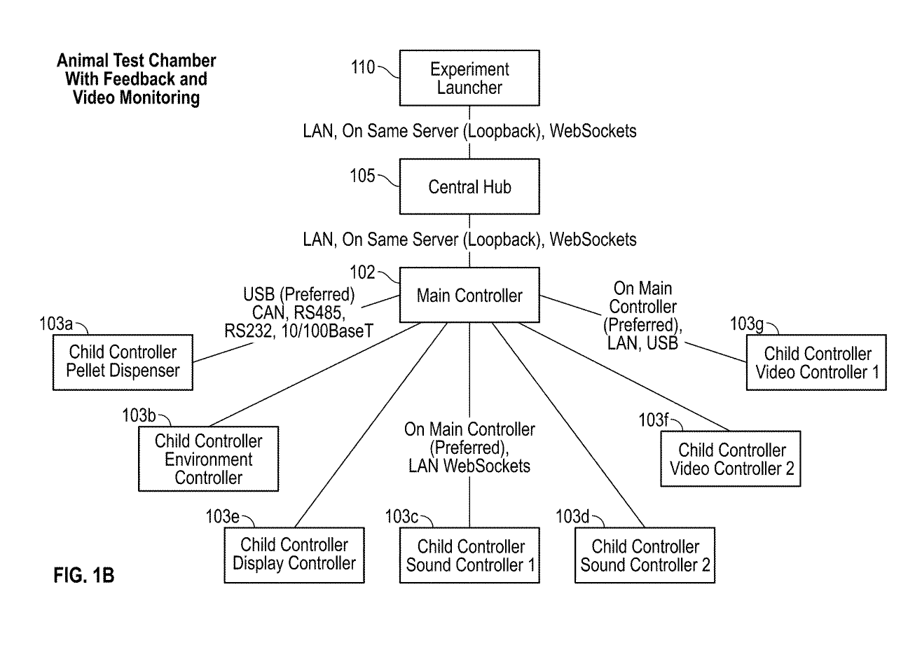

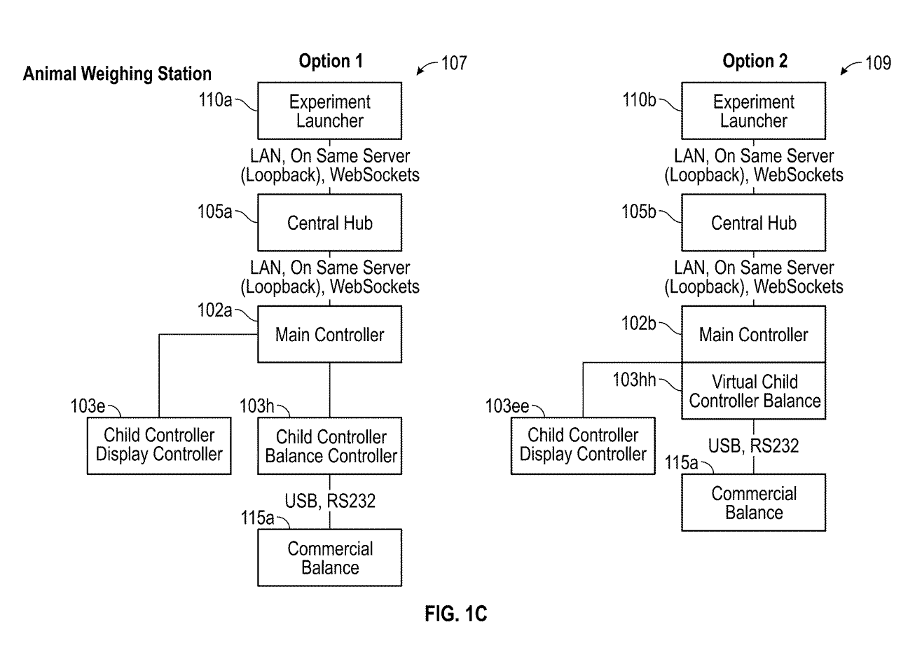

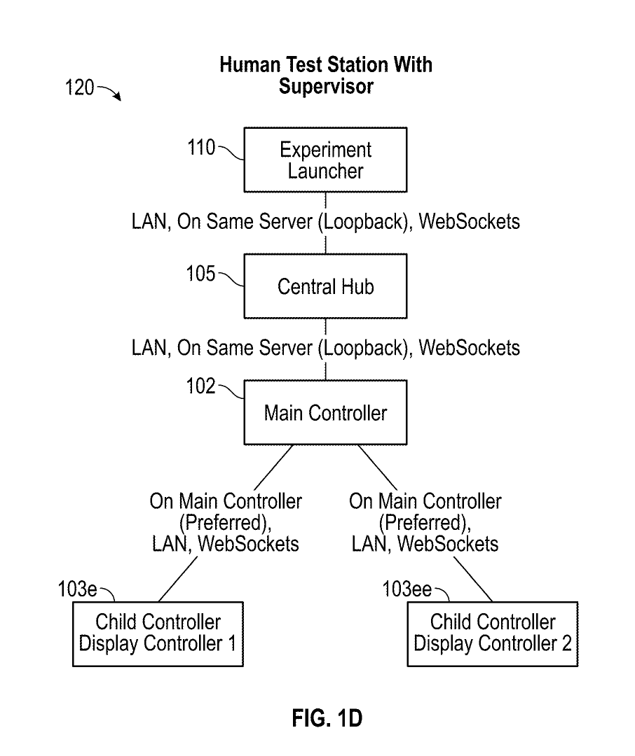

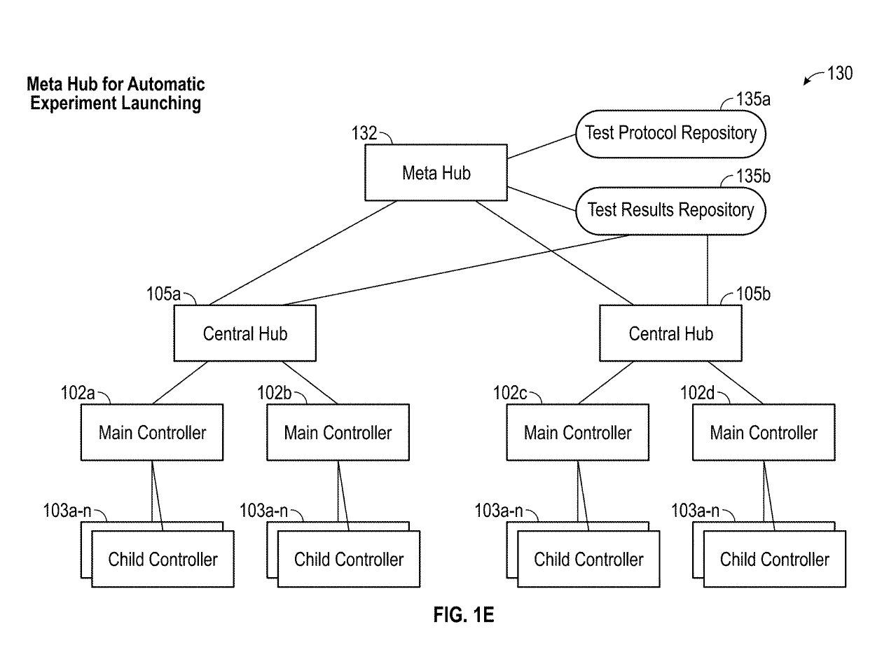

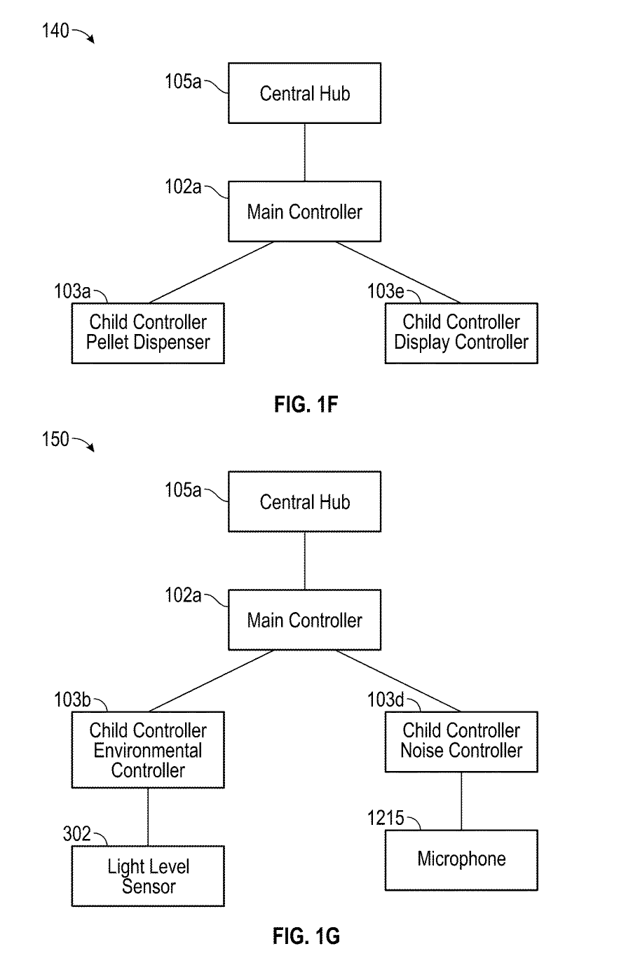

FIG. 1A depicts a system 101 for cognitive testing of a subject according to one embodiment. The illustrated embodiment 101 may include features useful in cognitive testing of non-human primates. Some examples of non-human animals that may be tested using the disclosed systems, methods and apparatus include mammals such as dogs, cats, rates, and primates, such as macaques. Invertebrates may also be tested in some aspects. For example, fruit flies may be utilized as test subjects in some aspects. The disclosed methods and systems may also be utilized in test environments utilizing human test subjects.

As shown, the system includes an enclosure 50 that may be comprised of a floor, a top portion, and one or more walls. The top portion may be supported by the one or more walls. The enclosure may also include an opening in one of the one or more walls, the opening being sized and shaped to allow a subject under test, such as a non-human primate, to enter the testing chamber 73. The enclosure 50 may be reinforced at one or more edges by reinforcement braces 30. The enclosure 50 may include a door 94, which is configured to selectively cover the opening.

A test subject, such as a non-human primate may be placed within a testing chamber 73 of the enclosure 50 during a test, and may be supported by a grate 74. The grate 74 may support the animal under test while allowing animal waste to fall below the grate to the bottom of the enclosure. The grate may be positioned within the enclosure below the interface and at a height that allows animal access to the interface 72, and also supplies sufficient room for animal waste to collect underneath during the duration of a test. For example, in some aspects, the grate 74 may be positioned at 1, 2, 3, 4, 5, 6, 7, 8, 9, 10, 11, 12, or 50 inches from the bottom of the enclosure, or at any position within that range.

The enclosure door 94 can be closed such that the testing chamber 73 may be environmentally controlled. The door 94 is shown in an open position in FIG. 1A. The door 94 may be secured by a latch 40.

Continuing with FIG. 1A, a testing interface 72 is located within the testing chamber 73. The testing interface 72 may include a display/touch interface 85. The display/touch interface 85 may include an integrated display device and/or touch screen device in some embodiments. In certain embodiments, the display/touch interface 85 may be configured to display one or more images to a subject under test. The display/touch interface 85 may, via an integrated touch screen for example, also detect when an animal touches the display/touch interface 85. A touch by the test subject may be detected in various environments using electrical capacitance, infrared, heat detection, or similar technology. In other embodiments, the display/touch interface 85 may detect touches using one or more force actuators. For example, a force measuring device may be positioned within the testing chamber 73. In certain embodiments, the display/touch interface 85 may be reinforced and/or protected by a durable but pliable coating or screen protector to increase its effective usage time. In certain embodiments, the display/touch interface 85 is configured to conduct a self-check to confirm that it is working properly.

In some aspects, the interface 72 may include an input device that receives input via a mouse for example. In these aspects, the input may take the form of a position change indication as the mouse is moved from a first position to a second position, or a button click of the mouse (such as a left, right, or center button click). In some aspects, the interface 72 may include a pointer input device. Input received in these aspects may indicate a position on the display/touch device 85 that was tapped or otherwise indicated by the pointer.

The interface 72 may also include a speaker 1515. In some aspects, the speaker 1515 may be used to generate one or more tones within the testing chamber 73. For example, the tones may be utilized to communicate correct and/or incorrect responses to challenges presented on the display/touch device 85.

The interface 72 may also include a pick-up area 802. The pick-up area 802 may provide a means for an animal under test to obtain a reward, such as a food pellet, from the testing environment upon satisfying particular test conditions. A particular test condition may include, for example, a correct response to a challenge presented on the display of the display/touch interface 85.

The interface 72 may also include an optional status or alternate feedback light 1504. In some aspects, the dispense light 1504 may be configured to illuminate when a reward, such as a food pellet, is dispensed to the pick-up area 802. In some aspects, the interface 72 may also include a light sensor 75. The light sensor 75 may be configured to determine a light level within the testing chamber 73 and provide the information to an environmental child controller circuit board, discussed below. In some aspects, the interface 72 may also include a screen camera 80.

A subject camera 45 may be mounted to the enclosure 50, for example, in the embodiment of FIG. 1A, the subject camera 45 is mounted to the top of the enclosure 50. The subject camera 45 may be configured to record images of the subject under test.

A second speaker 1220 may be mounted to the enclosure 50, for example, in the example shown in FIG. 1A, the speaker 1220 is mounted to the top of the enclosure 50. The speaker 1220 may be utilized, in some aspects, to generate white noise within the enclosure 50. The white noise may serve to reduce acoustical distractions emanating from outside the testing chamber 73.

A reward dispenser 95 is shown mounted to the top of the enclosure 50. The reward dispenser 95 may be configured to dispense a reward to a subject under test within the enclosure via the pick-up area 802.

The system 101 may also include four wheels 35 mounted to a bottom portion of the enclosure 50. The wheels may facilitate ease of movement of the system 101.

An environment of the testing chamber 73 may be electronically controlled. To facilitate the electronic control of the environment, the example embodiment of FIG. 1A shows a main controller 102 under a main controller cover 96.

In the illustrated embodiment, one or more child controller circuit boards 103a-g may be mounted to the top of the enclosure 50. The child controller circuit boards 103a-g may provide electronic control to various components of the system 101. For example, a display controller board 103e may provide electronic control of the interface 85. For example, the display controller board 103e may cause a display of the interface 85 to display one or more images to a subject under test within the enclosure 50. In some aspects, the reward dispenser 95 may be operably coupled to a reward controller 103a.

In some aspects, one or more measurement sensors may be positioned within the testing chamber. The measurement sensors may be configured to obtain measurements from the animal under test. For example, some aspects of the enclosure 50 may include a blood pressure measuring device, eye tracking device, force detector, or electroencephalography measuring device within the testing chamber 73. The blood pressure measuring device may be configured to measurement the blood pressure of an animal under test. For example, as the animal performs a test, their blood pressure may vary based on, for example, stress imposed on the animal by the test itself, a reward provided to the animal, or one or more environmental conditions of the test chamber 73. One of the child controller circuit boards 103a-g may be configured to control and/or receive data from the blood pressure measuring device. For example, blood pressure measurements may be received by one of the child controller circuit boards 103a-g and provided to a main controller 102, discussed below, in some aspects. In other aspects, including one or more of the eye tracking device, force detecting device, or electroencephalography measurement device, one or more child controller boards 103a-g may similarly be configured to control and/or receive data from these devices.

FIG. 1AA shows a modular architecture for cognitive testing of animals. The architecture can include multiple animal testing stations 101a-c. In some aspects, the system 101 depicted in FIG. 1A may be any one of the systems 101a-c shown in FIG. 1AA. Each animal testing station 101a-c includes at least a main controller 102. The main controller may include a hardware processor. The main controller 102 may include a modular bus architecture, including one or more modular bus interfaces that enable it to interface with a variety of ancillary devices, each of which may be under separate control. Each modular bus interface may provide for physical and electrical connection between the main controller 102 and a child controller 103a-g. The main controller's hardware processor may be configured to communicate with the one or more child controllers 103a-g by generating electrical signals across the modular bus interface(s). For example, via the modular bus interface between the main controller hardware processor and a hardware board processor on one or more of the connected child controllers, communication and control signals may be passed between a processor of the main controller 102 and a processor on the child controller(s) 103a-g.

Each child controller 103a-g connected to a main controller 102 may either receive input from, or generate output to, the enclosure 50 and specifically the testing chamber 73.

For example, child (or secondary) controllers 103a-g of FIG. 1AA are shown to include a reward dispenser 103a, environmental controller 103b, tone controller 103c, noise controller 103d, display controller board 103e, and video controller board in the illustrated aspect of FIG. 1AA. In various aspects, the number of child controllers 103a-n may vary. The controllers may be implemented as control circuit boards.

In some embodiments, one or more of the child controllers 103a-g may control other devices, such as weight scales (or balances), eye trackers, and any other devices associated with cognitive testing or training. In some embodiments, the child controllers coordinate rehabilitation devices, such as devices and equipment that help restore cognitive or motor function in individuals recovering from stroke, spinal cord injury, traumatic brain injury, or other neurological disorders. Such devices can be used for rehabilitation therapy in a clinic or at-home.

As shown in FIG. 1AA, each animal testing station 101a-c may be in communication with a central hub 105. The central hub 105 may provide information management and control for one or more of the animal testing stations 101a-c. In some aspects, the central hub 105 may be a computer that runs software configured to translate experiment protocols into hardware commands. The central hub 105 may include a main controller computer, such as a general purpose hardware processor, memory, and storage. The memory may store instructions that configure the processor to perform the functions discussed herein with respect to the central hub.

In some aspects, the main controller 102 may be coupled to or in communication with a server. The main controller computer may also be coupled to the testing stations 101a-c via a network that can communicate with the server. In some embodiments, the central hub 105 may include a web-based interface that is coupled to one or more servers that are coupled to the testing stations 101a-c.

In some aspects, device control functionality may be implemented on the main controller 102, e.g., a general purpose computer, instead of on a child controller 103. For example, some devices may benefit from a more fully featured processing environment which may be available on the main controller 102 as compared to a less sophisticated environment available on some child controllers 103. In some aspects, a display controller 103e may be an example of a component that benefits from implementation on and tighter integration with, the hardware available on the main controller 102. In some aspects, although a particular hardware component may be controlled via firmware and/or software executing on the main controller 102, the control software for this component may be implemented so as to be separate from other software also running on the main controller board 102. With this design, future architectures may make different choices, without necessarily being required to extensively rewrite or reconfigure the control software for that particular component. For example, in some aspects, the display controller 103e may be implemented as an object-oriented class that runs on the main controller 102 instead of as a separate circuit board. Another embodiment may choose to run the object oriented class on a separate child controller. While some changes may be required to adapt the object oriented class to the child controller environment, the number of changes required to make this transition may be reduced due to the original design's choice to implement the software for that controller in a modular way.

In some aspects, device control functionality may be implemented on the central hub 105. For example, in some aspects, the central hub 105 may directly coordinate a testing session for one or more of the testing stations 101a-c. For example, the central hub 105 may initiate commands for one or more of setting a noise level or temperature or humidity of an individual enclosure within a testing station 101a-c, displaying a prompt on an electronic display within a testing station 101a-c, or receiving an input response to a prompt via a touch device, or other input device, from a testing station 101a-c.

In some aspects, a test execution management process may be developed so as to run on either the main controller 102 or the central hub 105. For example, a python class may be developed that coordinates a testing session. Coordination of a testing station(s) 101a-c may include overall management and control of the session, indicating control of house lights, enclosure temperature (or humidity) and noise level, display of prompts and reception of test answers, dispensing of rewards, such as pellets, etc. The reward may include an edible reward such as a pellet, liquid, or paste. In other embodiments, an edible reward can include candy or other food items. In some implementations, the reward may be an inedible reward such as a toy, a coin, or printed material (e.g., coupon, sticker, picture, etc.). In some implementations, the reward may be experiential (e.g., song, video, etc.).

The python class may be combined and run on either the main controller 102 or the central hub 105 in some aspects. To enable this capability, each of the main controller 102 and central hub 105 may support a common set of APIs that provide for control of any of the physical child controllers 103a-g, from either the main controller 102 or the central hub 105. When run from the central hub 105, the APIs may provide for an ability to specify which of the testing stations 101a-c are being controlled. When the test execution management process is run from a particular main controller 102, there may be no need to specify which testing station 101a-c is being controlled.

In some aspects, device control functionality may be implemented on the mother board of the main controller 102 instead of on a child controller 103e-f. For example, some devices may benefit from a more fully featured processing environment which may be available on the main controller 102 as compared to a less sophisticated environment available on some child controllers 103e-f. In some aspects, a display controller 103e may be an example of a component that benefits from implementation on the hardware available on the main controller 102.

In some aspects, although a particular hardware component may be controlled via firmware and/or software executing on the main controller 102, the control software for this component may be implemented so as to be separate from other software also running on the main controller 102. With this design, future architectures may make different choices, without necessarily being required to reinvent the control software for that particular component. For example, in some aspects, the display controller 103e may be implemented as an object-oriented class that runs on the main controller 102. Examples of such logical controllers are described below. In other embodiments, the display controller 103e can be a physical child controller.

In certain aspects, device control functionality may be implemented on the central hub 105. For example, in some aspects, the central hub 105 may directly coordinate a testing session for one or more of the testing stations 101a-c. For example, the central hub 105 may initiate commands for one or more of setting a noise level or temperature of an individual enclosure within a testing station 101a-c, displaying a prompt on an electronic display within a testing station 101a-c, receiving an input response to a prompt via a touch device, or other input device, from a testing station 101a-c.

In some aspects, the main controller 102 includes a processor and a non-transitory computer readable memory containing instructions for one or more cognitive tests. That is to say, in certain embodiments the exact steps in for one or more cognitive tests are stored at the main controller level 102 and not at the central hub level 105. Such tests may be updated or changed. In this way, the central hub 105 may be used to simply initiate a cognitive test selected form a plurality of pre-programed tests that reside in the memory of the main controller 102. The main controller 102 can send then instructions to one or more child controllers 103 which execute the commands.

One or more sensors can be configured to detect and/or confirm that the command was executed correctly. For example, the main controller 102 may instruct the reward controller 103a to dispense a reward. A sensor may be configured to confirm that a reward was indeed dispensed. If an error is detected, the sensor may inform the reward controller 103a and/or the main controller 102 and appropriate action may be taken.

In some aspects, a test execution management process may be developed so as to run on either the main controller 102 or the central hub 105. For example, a java class may be developed that coordinates a testing session. Coordination of a testing station(s) 101a-c may include overall management and control of the session, indicating control of house lights, enclosure temperature and noise level, display of prompts and reception of test answers, dispensing of rewards, such as pellets, etc.

In some aspects, a high level programming language may be utilized to control the main controller 102. For example, in some aspects, the main controller 102 may execute a java virtual machine. In these aspects, a java class may be compiled and run on either the main controller 102 or even on the central hub 105 in some aspects. To enable this capability, each of the main controller 102 and central hub 105 may support a common set of APIs that provide for control of any of the child controllers 103a-g, from either the main controller 102 or the central hub 105. When run from the central hub 105, the APIs may provide for an ability to specify which of the testing stations 101a-c are being controlled. When the test execution management process is run from a particular main controller 102, there may be no need to specify which testing station 101a-c is being controlled.

### Modularity

One of the most important design principles for this system is modularity. More specifically, the system was designed such that all of the components could work independently of one another. This design improves the system's stability because each function performed by the testing environment can work without interference from other functions. Furthermore, each individual component can be easily replaced or added without affecting any other subsystem This allows the system to be easily adapted to various different experiments or even entirely different testing environments.

Using this system, researchers can devise new experiments without being constrained by the system architecture. To make the testing unit modular, the unit was divided into various subsystems, called child controllers 103a-n. The child controllers 103a-n may comprise Arduino microcontrollers. Arduino microcontrollers are robust enough to allow it to perform a variety of functions while still being common and easy to program. By using Arduinos for most subsystems, the entire testing environment is greatly simplified while still allowing for highly specialized child controllers 103a-n. The Arduino uses USB serial communication, allowing it to interface with any main controller 102. This modularity makes it easy to construct and maintain each individual subsystem. Arduinos are readily available for purchase and can easily be fitted with small printed circuit boards (PCBs), also known as shields, which will handle the electronics necessary by the system. To build or repair the system, one can upload the correct program to the Arduino and install the appropriate shield to the top of the Arduino.

Components that affect the testing environment, such as speakers or LED lights, can connect to the PCB shields with detachable Japan Solderless Terminal (JST) or Molex connectors. The ease of attaching or detaching component makes the system easier to maintain. JST and Molex connectors do not require any tools to attach. Furthermore, the connectors are chosen so that it is difficult to plug components in incorrectly. For example, it is impossible to plug in a JST or Molex backward. Such connectors help to fulfill the goal of modularity since they allow for any compatible components to be installed in their place. For example, if a future project required a different sized speaker, the shield could remain unchanged and the new speaker would simply plug into the appropriate Molex headers. To further aid with maintenance, all of the subsystems Arduinos may be mounted onto a removable polyvinyl chloride (PVC) plate. In certain embodiments, this plate consists of four mounting locations where each Arduino can simply plug into the PVC plate.

Although some aspects of the methods, apparatus, and systems described herein are directed to testing environments for non-human primates, other aspects of the systems, methods and apparatus could very easily function as a testing environment for various other potential test subjects (e.g. rodents, humans, etc.) by adapting some of the current child controllers 103 or introducing new child controllers. For instance, adapting the system to perform rat testing may be accomplished in some aspects by adapting the firmware so that the child controllers 103 can interface with the new hardware. This means that the Arduino implementation of some commands may change to ensure functionality over the interface between the Arduino and rat specific hardware. If the new rat-specific hardware utilized many components common to the testing environments provided as examples herein, the system would require almost no change.

### Main Controller

The main controller 102 is configured to be the head of each testing unit. In certain embodiments, the main controller 102 runs on a personal computer along with the logical child controllers 103. The main controller 102 can be configured to accept requests from a central hub 105 and, in turn, to dispatch commands to the appropriate child controller 103. The main controller 102 may also send feedback to the central hub 105 such as touch screen input form the test subject, system health checks, and status updates.

The main controller 102 is generally configured to instruct one or more child controllers 103 from the central hub 105. For example, when the central hub 105 sends a “dispense” command, the main controller 102 is asked to dispense a reward. In turn, the main controller 102 may be configured to delegate this task to the appropriate child controller 103a (e.g. the reward controller).

The main controller 102 may be configured to communicate with one or more child controllers 103a-n using predefined serial Universal Asynchronous Receiver/Transmitter (UART) messages. For example, passing the string “60%” can set either the light or sound level to 60% intensity. This standard allows for additional controllers to be easily added if an experiment or testing protocol needs functionality that is currently not provided. As long as the new subsystem follows the established standards, the main controller 102 will only need small changes to communicate with the new child controller 103a-n. Furthermore, adding this new child controller 103a-n would have no effect on any of the other subsystems.

In some aspects, the main controller may be configured to automatically detect whether a child controller circuit board is installed in one or more modular interface connectors attached to the main controller circuit board. For example, in some aspects, a main controller 102 may transmit a command over one of its modular interface connectors and then wait for a response to be received within a predetermined time period. If a response is received within the time period, then the main controller may determine a child controller circuit board is installed on that modular interface connector. If no response is received, the main controller may determine that no child controller circuit board is installed on that modular interface connector. Thus, in some aspects, the main controller 102 may sense the presence or absence of the child controller circuit board by generating electrical signals over a modular interface connector, the electrical signals defining a command, and sensing whether a response to the command is received over the second modular interface connector, and adapting an animal testing capability based on the sensing.

### Logical Child Controllers

In certain embodiments, the child controllers 103a-n may comprise logical child controllers as opposed to physical child controllers. For example, the child controllers 103a-n may be Python classes, which are controlled by the main controller 102. That is to say, the logical child controllers may include separate logical blocks that run on the same physical hardware as the main controller 102. Thus, in certain embodiments, the child controllers 103a-n run on the same PC as the main controller 102. The functions performed by the child controllers 103a-n may be handled by the classes themselves. In this way, the main controller 102 may simply make function calls to interact with the child controllers 103a-n. Thus, the main controller 102 may pass the actual functionality to a distinct child controller 103a-n.

Exemplary logical child controllers 103 may include a display controller 103e and/or a video controller 103f The display controller 103e may be configured to control a touchscreen (e.g. a display screen 185a and a touch sensor 185b) and the video controller 103f may be configured to control video recording and/or processing. In certain embodiments, the display controller 103e runs in its own thread on the main controller 102. In certain embodiments, the display controller 103e includes a monitor, which displays images during the session as well as the touch input, which provides feedback on which image the subject touched. Similarly, in certain embodiments, the video controller 103f runs in its own thread on the main controller 102. In certain embodiments, the video controller 103f includes one or more video recording devices, which record all or some of a testing session.

### The Environment Controller

The environment controller 103b may be configured to control various aspects of the environment, such as internal lighting, temperature, fan speed, ambient noise, and the like. In certain embodiments, the environment controller 103b is configured to control the non-auditory aspects of the cognitive testing environment. For example, the environment controller 103b may be configured to control one or more dimmable lights. In certain embodiments, the environment controller 103b is configured to control a separate indicator light that is designed to notify a test subject that the test has begun. In certain embodiments, the environment controller 103b is configured to control a fan disposed within the testing chamber to circulate air through the environment to prevent temperature spikes and/or carbon dioxide buildup. In certain embodiments, the environment controller 103b is configured to monitor the status of a mechanical lever (not shown) that a subject may response to depending on the cognitive procedure that is being performed.

In some aspects, the environment controller 103b may be configured to monitor the relative times and or time periods when a fan or other HVAC system is operating. Noises generated by such systems may cause distractions to testing subjects and/or may skew response times and/or results. The environment controller 103b may further be coupled to one or more sensors for monitoring relative noise levels, light levels, temperature levels, wind speed, and the like. In this way, a more controlled and consistent testing environment may be established and confirmed. For example, the main controller 102 may instruct the environment controller 103b to set the temperature within the testing chamber to a particular temperature or temperature range. A temperature sensor may be configured to determine the actual light level within the testing environment. The temperature sensor may communicate directly with the environment controller 103b and/or directly with the main controller 102. In some embodiments, the environment controller 103b may confirm the temperature level by relaying information from the temperature sensor to the main controller 102. For example, if the HVAC systems are malfunctioning and/or deteriorated over time, when the environment controller 103b commands a certain temperature or temperature level, the temperature may not in fact reach the commanded level. In some aspects, if the HVAC systems are malfunctioning and/or deteriorated over time, the systems may run for longer periods of time than with previous testing, providing an environmental variable (e.g. longer periods of noise from the HVAC) that may now be determined and potentially compensated for to provide a uniform testing environment each during each test.

### Display Controller

In some embodiments, a display controller 103e is coupled to the display interface 185 which may include a touch screen configured to receive touches from a subject. In certain embodiments, the display controller 103e is implemented on the main controller 102 rather than an Arduino because, the display controller 103e may require a fully featured computer to be better suited for displaying images on a screen. In order to keep the goal of modularity, the main controller 102 may implement the display controller 103e as a separate class, which is instantiated. While the control mechanism for the display controller 103e may run on the same physical hardware as the main controller 102, the two systems may be logically separated. The separation of functionality into the display controller 103e may enable the main controller 102 code to be less complicated.

To handle display processing and input/output (I/O) from the touchscreen, the main controller 102 may use Pygame. This library may provide a simple cross-platform interface to a Simple DirectMedia Layer (“SDL”) library. Pygame may be used to display images onto the screen. Pygame is an open source library built on top of Python made specifically for graphics applications. Pygame is multi-platform, thus it was found to be advantageous because it interfaces with many different graphics handlers such as X11 or framebuffer allowing the display controller 103e to run on a variety of computer architectures, including single board computers such as a Raspberry Pi device. However, Pygame is not thread safe, meaning that the module has to be run by the main thread, otherwise unexpected errors may occur. Therefore, the main thread may be configured to launch the display controller 103e, which uses the Pygame library. Having the Pygame event listener loop running on a separate thread is also an option but may result in a less stable system.

As mentioned above, the display controller 103e may be a Python class whose constructor initializes Pygame. The class may have two methods: showImages( ) and loop( ). The display controller 103e may call the showImages( ) when the TCP request handler thread receives a show image command. The method may then load specified images into the Pygame surface and update the display. The event listener, loop( ), may be called at the end of the main( ) function. This may be configured as an infinite loop listening to Pygame events like mouse clicks or program termination. Thus, in certain embodiments, when a mouse click is detected or screen touch is detected, the pixel and/or screen coordinates may be sent back to the open TCP socket connection. The display controller 103e may also determine which displayed image, if any was selected and/or touched by a test subject.

Images displayed by the display controller 103e may be stored locally with a display controller class. In this way, the main controller 102 can simply tell the display controller 103e which image to display rather than sending the entire image to the display controller 103e. Any images that are stored on the local machine can be used with the display controller 103e since the controller may simply reads from a file rather than creating an image itself. Thus slide decks of images used during an experiment may be easily changed without having to change the display controller 103e. The display controller 103e may be configured to display png, jpg, and bmp images; pdf, doc, and ppt files; and/or other types of image formats or other formats able to display images. In some embodiments, a plurality of images is displayed on the touch screen. A subject is rewarded if the correct image is touched.

### Video Controller

The video controller 103f may be used to monitor an experiment while it is in progress. In certain embodiments, the video controller 103f may control one or more video recording devices. In certain embodiments, the video controller 103f can control an environment camera 75 (in FIG. 1A) positioned such that the entire testing environment may be recorded to provide researchers with a general overview of what the subject is doing during a test. The video controller 103f may be further configured to control a screen camera 80 (in FIG. 1A) positioned above the touch screen to provide a more detailed view of the subject's interaction with the display. In this way, researchers can verify the experiment in real time or at a later date to ensure that the received data corresponds to the subject's actions.

In certain embodiments, the cameras used to capture video of the testing environment are Logitech C920 USB webcams or other similar plug-and-play cameras known in the art. This may allow the cameras to be installed using minimal overhead. For example, the Logitech C920 also has a built-in hardware H.264 encoder, which outputs HD video. Such hardware encoding may reduce the size of the data stream and lesson the processing load on the main controller 102 which does not have to process the video before sending it to the central hub 105. In this architecture, the video controller 103f may initialize a stream and passes it directly to the central hub 105. In this way, almost all of the processing work is removed from the main controller 102, making the video controller 103f independent of the main controller 102. Such a configuration also reduces the required bandwidth for communicating with the central hub 105.

In certain embodiments, the video controller 103f may create a constraint for the hardware running the main controller 102. This is because HD video streaming in real time requires a lot of bandwidth, even with encoding, the computer running the main controller 102 needs to have a sufficiently large data bus. Thus, hardware connections may include two USB connections each handling a stream into the main controller 102 and an Ethernet connection between the main controller 102 and the central hub 105, which can transmit both streams as well as all other communications.

In certain embodiments, the main controller 102 may be configured to interface with one or more web-based systems (e.g. using VLC, an open source, multiplatform media player available at www.videolan.org as of the filing date of this application). VLC is available for a wide variety of systems, including PC, Mac and Linux using both x86 and ARM computer architectures. In this way, video streams may be broadcast using, for example, Real Time Streaming Protocol (RTSP), allowing the central hub 105, or any connected client, to view the video in real time or at a later date from a remote location. The central hub 105 may provide a port on the main controller 102 to broadcast each video stream. The central hub 105 may access the streams by listening to those ports at the IP address of the main controller 102.

### Physical Child Controllers

Physical child controllers (e.g., 103a-g) may be configured to be subordinate control units overseen by the main controller 102 in the system architecture. In certain embodiments, the physical child controllers 103a-g include Arduino microprocessors with a PCB connected on top. The PCBs may then connect to the different hardware pieces of the testing environment. Each child controllers 103a-g may be mapped to a distinct subsystem based on its function. In this way, each command may correspond to one of child controllers 103a-g and multiple commands can be smoothly executed at the same time by each of child controllers 103a-g.

In some aspects, each of child controllers 103a-g may include the same general architecture. The child controllers 103a-g may also share command interpretation. That is to say, in certain embodiments, each child controller 103a-g is configured to receive commands from the main controller 102 using serial communication via a USB UART for example. Such commands may take the form of a single ASCII character that the child controller 103a-d has been programmed to recognize. The child controllers 103a-g may also be configured to communicate back to the main controller 102. The child controllers 103a-g may be programmed to receive an identify command and reply with the type of controller they are. The child controllers 103a-g may also receive and interpret sensor data in order to return the completion status of a command.

FIG. 1B is an example configuration utilizing the modular architecture of FIG. 1AA. The configuration of FIG. 1B includes the central hub 105, main controller 102, and child controllers 103a-g. In some embodiments, as shown, an experiment launcher 110 may be in communication with the central hub 105 over a network, such as a LAN. Alternatively, the experiment launcher 110 and central hub 105 may be collocated on the same computer, such as a server. In some aspects, the communication between the central hub 105 and the experiment launcher 110 may be performed via a socket-based or HTTP over TCP/IP connection, and thus when the two components are collocated a loopback connection may be employed.

Similarly, communication between the central hub 105 and the main controller 102 may be performed over a local area network (LAN) in some aspects. In certain embodiments, communication between the central hub 105 and the main controller 102 may utilize a socket-based connection, thus both separate installations and collocated installations of the central hub 105 and main controller 102 may be supported.

In certain embodiments, as illustrated in FIG. 1B, the main controller 102 may be in communication with the child controllers 103a-g via a variety of interface technologies, including USB, CAN, RS 485, RS 232, 10/100 Base T. In some aspects, the main controller 102 may communicate with a first child controller via a first interface technology, such as USB, and communicate with a second child controller via a second interface technology, such as 10/100 Base T.

As noted in FIG. 1B, in some aspects, a child controller 103 may not be a physical circuit board, but may instead be a set of software instructions stored in a memory that configure an electronic hardware processor to perform functions associated with the child controllers discussed herein. In these embodiments, a child controller may be virtualized so as to run on the main controller 102 hardware. For example, control of some physical hardware may not require dedicated components, but can be accomplished by hardware already present on the main controller 102. In these embodiments, one or more child controller(s) 103a-g may run as part of the main controller 102. For example, a software module (not shown) may be configured to control a first hardware component. The software module may be further configured to run on the main controller 102 in some aspects, and on a separate child controller 103 in other aspects. In some other embodiments, processor instructions implementing a child controller may be configured to run within a web browser, for example, as a browser plug-in. Alternatively, in some aspects, the processor instructions may be implemented using JavaScript 5. A child controller implemented using JavaScript 5 may run within a browser that fetches a document including the child controller HTML document and the JavaScript controller code. Alternatively, the child controller may run within a software environment that processes JavaScript 5 in these aspects. In some cases this may be a traditional “browser,” however, the disclosed embodiments contemplate other non-browser JavaScript 5 processing engines as well. In some aspects, the browser or other child controller execution environment may itself run on the main controller hardware, or it may run on a separate computer, that communicates with the main controller over a network.

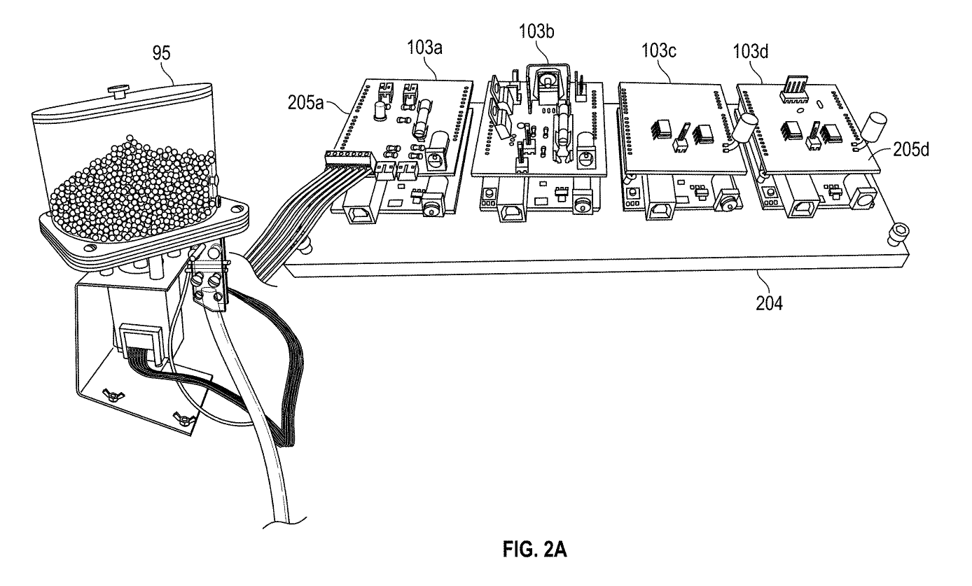

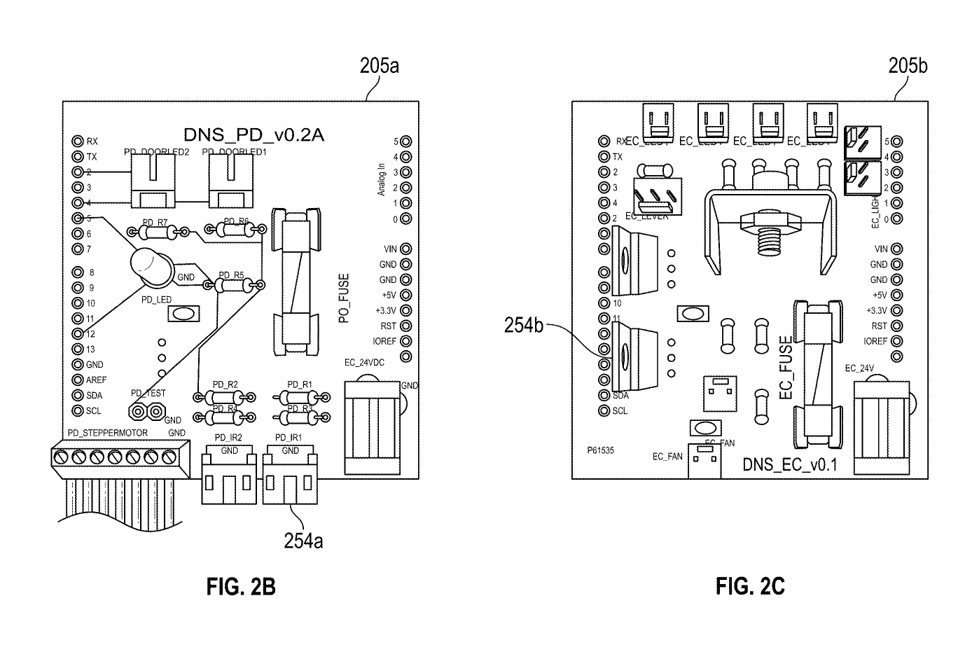

In some aspects, a virtualized child controller and a child controller implemented on a dedicated circuit board, such as those shown in FIG. 2A below, may utilize an equivalent messaging interface to communicate with the main controller 102. For example, the syntax and protocols used to send commands to the physical child controllers and virtual child controllers may be equivalent or identical in some aspects. For example, in some aspects, a messaging infrastructure may utilize a layered model. When communicating to a physical child controller, a first communication layer may provide for communication across the physical bus and modular interface between the main controller 102 and child controller 103. Above the first communication layer may be a second communication layer, for example, the Internet Protocol may be used in some aspects as the second communication layer. This second communication layer may be common for both communication between the main controller 102 and a physical child controller 103, and between the main controller 102 and a virtualized child controller 103.

FIG. 1C shows two further example configurations utilizing the modular architecture of FIG. 1AA. FIG. 1C shows a first configuration 107 including an experiment launcher 110a, central hub 105a, and a main controller 102a. The main controller 102a is in communication with two child controllers, a display controller 103e and a balance controller 103h. The child controller 103h is in communication with a commercial balance 115a. The experiment launcher 110a may be a hardware computer including a hardware processor and hardware memory (not shown). The hardware memory may store instructions that configure the hardware processor to perform one or more functions associated with the experiment launcher. The experiment launcher may, for example, start sequences of testing commands, display status of the test sequences as they run on a management console separate from the test stations themselves, and record the results of the tests in a data store, such as a test result repository database (also not shown in FIG. 1C).

In some aspects, the experiment launcher also records information about animal subjects in a particular test. For example, the experiment launcher may record one or more of a subject name, an identifier number, a weight, a sex, or a species. In some aspects, the experiment launcher may obtain the subject information from a test subject database. In some aspects, before an animal is placed in a test enclosure, a barcode attached to the animal (for example, via a neck or wrist band) is scanned. In some aspects, the animal may be tattooed with the barcode. Alternatively, in some aspects, a fingerprint reader or iris scanner may obtain the identification from the animal. The barcode information is retrieved by the experiment launcher and used as a key to search the test subject database, which returns information relating to the test subject, for example, as discussed above. This information may then be stored along with the test results in the test result repository database. In some aspects, the test results may be uploaded from the main controller 102 and/or the central hub 105. By storing the subject information with the test results, the ability to archive the test results and review a complete and comprehensive record of the test is better assured. In some aspects, the experimental launcher may parse the test results before storing them in the test result repository. For example, the results may be parsed into test result values that are suitable for display and analysis to scientists. These results may then be either displayed on an electronic display or stored in the test result repository in their parsed form.

In some aspects, the experiment launcher may aggregate test results from multiple tests, for example, from the same test system or across multiple test systems, and generate one or more reports, or test result values based on the aggregated data. For example, in some aspects, the experiment launcher 110a may determine a number of animals across a set of n tests that acknowledged or answered a particular challenge correctly. A percentage of animals successfully answering the challenge may then be determined by the experiment launcher 110a. This aggregated result may then be stored to the test result repository and/or displayed on an electronic display.

In some aspects, the experiment launcher communicates with the central hub over a web sockets connection, for example, using either TCP or UDP protocol for the communication. In some aspects, the experiment launcher and central hub may run on the same physical computer. In these aspects, a loopback TCP connection may be utilized for communication.

FIG. 1C also shows a configuration 109. The configuration 109 includes an experiment launcher 110b, central hub 105b, and main controller 102b. Whereas the balance controller 103h included hardware separate from the main controller 102a in the configuration 107, in the configuration 109, a virtual balance child controller 103h h runs on the main controller hardware 102b, and is thus virtualized within the main controller 102b. In other embodiments, any other child controller can be virtualized on the main controller.

FIG. 1D shows another example configuration utilizing the modular architecture of FIG. 1AA. FIG. 1D shows a configuration 120 that includes the experiment launcher 110, central hub 105, main controller 102, a display controller 1 103e, and a display controller 2 103ee. In the configuration 120 of FIG. 1D, the two display controllers 103e and 103ee are implemented differently. The display controller 103e may be implemented in a web browser. In certain embodiments, the display controller 103e may utilize JavaScript and/or Web Sockets may be utilized in a QT application (The QT Company of Finland http://www.qt.io), PyGame (www.pygame.org) or some other graphical user interface tool kit, or other user interface, including any human-control interface.

The child controller 103ee may be provided via a display port on the main controller 102, a tablet (not shown), a smartphone (not shown), or a separate computer (not shown). More generally the child controller may be provided via a port on any suitable client or end user portable electronic communication device.

FIG. 1E shows another example configuration utilizing the modular architecture of FIG. 1AA. FIG. 1E shows a configuration 130 including a meta hub or meta hub processor 132, test protocol repository 135a and a test results repository 135b, two central hubs 105a-b, four main controllers 102a-d, and up to an arbitrary number “n” controller boards 103a-n. In some embodiments, the meta hub can reside in the cloud, in a server room, or on a remote computer. The central hub and main controller can reside on the local computer in each testing station and interface electronically with the child controllers by a USB port or similar connector. In the configuration 130, the meta hub 132 may provide for coordination of multiple dynamically selected tests for each subject. For example, in certain embodiments, one or more main controllers 102 may generate test result data. The test result data may be communicated from the main controller(s) to the central hub 105a or 105b and optionally to the meta hub 132. The meta hub 132, or in some other embodiments one of the central hubs 105a-b, may then determine a next action based on the test results. For example, in some aspects, one or more of the meta hub 132 and/or the central hub(s) 105a-b may determine a next test or experiment to perform based on the test results.

In some aspects of the configuration 130, the central hubs 105a-b may be configured with the ability to run scripting language files. For example, in some aspects, the central hubs 105a-b may be configured to run scripts including sequences of testing commands to change the configuration and/or state of testing hardware, as described in further detail herein. In some implementations, the scripts may be expressed in a domain specific language.

In some aspects of the configuration 130 the meta hub may be configured with the ability to run scripting language files. Some of these scripts may be “study” scripts, which may control the execution of multiple tests that are part of the study. The “study” script may also indicate conditional logic that varies which tests are run as part of a study based on results of particular tests. In some implementations, the scripts may be expressed in a domain specific language.

The meta hub 132 may be configured as a repository of logic to implement a study. The study may be comprised of a plurality of tests. When the meta hub 132 receives a query from the central hub 105 as to which test should be run, the meta hub 132 may consult a database defining one or more studies, and parameters passed to it from the central hub, such as a subject name. An exemplary database is the test protocol repository 135a. The meta hub then determines which particular test should be run by the requesting central hub 105. This information is then passed back to the central hub 105a. The meta hub 132 may also provide a test script for execution by the central hub 105 to the central hub 105. The central hub 105 then executes the test script provided by the meta hub 132.

In some aspects of the system 130, the meta hub 132 may be configured with the ability to run scripting language files. For example, in some aspects, the meta hub 132 may be configured to run scripts. Some of these scripts may be “study” scripts, which may control the execution of multiple tests that are part of the study. The “study” script may also indicate conditional logic that varies which tests are run as part of a study based on results of particular tests.

After a “study” script is launched on the meta hub, one of the central hubs 105a-105b, may query the meta hub 132 for information on which specific test should be performed as part of the study. As one implementation, the study script can be a Zaius script. The study script will handle this request and respond.

The script executed by the central hub 105 may cause the central hub 105 to send commands to one or more of the child controllers 103 and receive results back from the child controllers 103. Results of the various commands performed by the test script may be stored in a log file. After the test script completes, the central hub 105 sends test result logs back to the meta hub 132 to be saved. The central hub 105 then requests additional test script(s) from the meta hub 132. The meta hub 132 may then determine whether there are additional test scripts for the central hub 105 to run, or if the testing is complete. This information will then be passed back to the central hub 105.

FIG. 1F shows another example configuration 140 utilizing the modular architecture of FIG. 1AA. The configuration 140 includes a central hub 105a, a main controller 102a, a pellet dispenser controller or reward dispenser controller 103a, and a display controller 103e.

In some aspects, the central hub 105a may communicate with the main controller 102a, which in turn communicates with the two child controllers 103a and 103e to control the a reward dispenser and an electronic display. For example, in some aspects, a DSL script including sequences of test commands may run on the central hub 105a. The DSL script may include a command to initiate a reward dispense command. The reward dispense command is sent from the central hub 105a to the main controller 102a. Upon receiving the reward dispense command, the main controller 102a looks up the command in a configuration table, and determines the appropriate command syntax for the reward dispense command that can be forwarded to the child controller 103a. The child controller 103a then executes the command, and sends a command complete indication to the main controller 102a. The main controller 102a forwards the command complete command to the central hub 105a. The central hub 105a triggers an event, such as specified in the sequence of commands included in the DSL script, upon receiving the command complete indication. This causes an event handler within the DSL script to be executed. By this method, application level DSL code can be specified for execution in the script to handle completion of the command.

In contrast with the synchronous reward dispense command processing described above, some aspects may utilize a more event driven, asynchronous (e.g., unsolicited event; triggered) processing model. For example, control of touch inputs from an electronic display may be asynchronous in nature. For example, when a touch event occurs on an electronic display, the child controller 103e may generate an asynchronous notification event for the main controller 102a. The main controller 102a may forward the new event to the central hub 105a. The central hub 105a then triggers an event handler in a DSL script. If the application level script includes a handler for the event, control may be transferred to the application level code, which may handle the event processing. If no application level handler is defined for the event, the system may provide a default event handler for touch events. For example, the default touch event handler may perform no operation upon receiving the touch event, or may log the event.

FIG. 1G shows another example configuration 150 utilizing the modular architecture of FIG. 1AA. FIG. 1G shows a configuration that utilizes a central hub 105a, main controller 102a, environmental controller 103b, and a noise controller 103d. The environmental controller 103b is physically connected to a light level sensor 302 and the noise controller 103d is physically connected to a microphone 1215.

In some aspects, the central hub 105a may communicate with the main controller 102a, which in turn communicates with the two child controllers 103b and 103d to control the light sensor 302 and microphone 1215. For example, in some aspects, a script (e.g., Zaius script) including sequences of test commands may run on the central hub 105a. The script running on the central hub 105a may initiate a calibration command (as an example command) and communicate this calibration command to the main controller 102a. As part of the example calibration command, the main controller 102a may command the environmental controller 103b to turn on lights at a predetermined level. The main controller 102a then requests a light level measurement be made by the environmental controller 103b. An adjustment to the light level (either up or down) may then be made based on the light level measurement. The main controller 102a may then request a further light level measurement from the light level sensor 302 via the environmental controller 103b. Depending on the results, the light level may be adjusted up and/or down. This cycle may be repeated until an acceptable light level is achieved. The main controller 102a may then send a calibration set point to the central hub 105a. The central hub may store the set point and use one or more stored set points for subsequent tests. White noise sound levels and tone sound levels may be calibrated in a similar manner. The microphone 1215 on the noise controller 103d can be used to set the level of white noise in some aspects. The same microphone 1215 can be used in some aspects to sense tones generated by the tone controller 103c. The main controller 102a can coordinate the two controllers 103b and 103d to allow the microphone 1215 on one controller to help set the level on a second controller.

In some aspects, an asynchronous event processing model is used. For example, again referring to FIG. 1G, the child controller 103b may sense that a light level detected by the light level sensor 302 is too low (below a threshold). The child controller 103b may generate an event notification based on the low light level. The event notification may be sent by the environmental controller 103b to the main controller 102a. The main controller 102a may forward the event to the central hub 105a. The central hub 105a may then invoke an event handler defined by a script running on the central hub 105a. The event handler may be invoked in response to receiving the event from the main controller 102a. The event handler may be defined in an application level script, such as a

DSL script (e.g., Zaius script) including a sequence of test commands implementing the desired test protocol and/or test event handling protocols. In this example, control may then be transferred to the script to continue processing the event. For example, in some aspects, a script event handler may then adjust the light level up, or may abort a test if the light level is such that the results of a running test may be corrupted by the low light level.

FIG. 2A shows a reward dispenser 95 and examples of the child controllers 103 shown in FIG. 1AA. As discussed above, the child controllers 103a-g may be designed to control a variety of devices, including, for example, the reward dispenser 95 (via controller 103a), an enclosure environment (via controller 103b, which may control devices such as fans and heaters), tone generators (via controller 103c), and/or enclosure noise levels (via controller 103d).

Each child controller 103a-g discussed above may be mounted to a polyvinyl chloride (PVC) Plate 204. The plate 204 may include one or more mounting locations. Each mounting location may be configured to secure a physical child controller. For example, in some aspects, each mounting location may comprise one or more nylon standoffs that provide for screwing the physical child controller(s) down to the nylon standoffs. In some embodiments, the PVC plate 204 may be configured to be removed from the system 101 in one piece to aid with system maintenance. In some embodiments, the PVC plate 204 may be configured to be removed from the system 101 in one piece to aid with system maintenance. In another embodiment, a larger plate may include in addition to the child controllers 103a-n, the noise speaker, enclosure lights, video cameras, and microphone 1215.

Each child controller 103a-g may also include a bus interface for communication with the main controller 102, illustrated above in FIG. 1AA. In some aspects, a Universal Serial Bus (USB) can be used for communication between any of the physical child controllers 103a-g and the main controller 102. Other bus architectures are also contemplated.

In some aspects, a hardware architecture across the child controllers 103a-g may be similar or identical. This may provide for reproducibility of results and provide for a reduced cost to maintain the multiple physical child controllers. In some aspects, the physical child controllers 103 may be implemented with any microprocessor. In some aspects, an Arduino microcontroller may be used. The Arduino is a robust microcontroller than can perform a variety of functions while still being easy to obtain and program. Use of the same microprocessor for multiple physical child controllers simplifies the testing environment. Despite the commonality across hardware for the different physical child controllers, each child controller can still be dedicated to a particular component based on the firmware developed for each child controller.

For example, the Arduino microprocessor in particular can be connected to printed circuit boards (PCBs) that are also referred to as shields 205a-d (205b-c not indicated in FIG. 2A for clarity). The shields 205a-d may be customized with hardware necessary to perform a particular task, such as control of a particular device. An appropriate firmware program can be uploaded to an Arduino processor resident on one or more of the shields 205a-d. The firmware may enable the Arduino to control the dedicated hardware provided on the connected shield.

The child controllers 103a-d may also share a common interface with the main controller 102 (as shown above, e.g., in FIG. 1AA). In some aspects, serial communication with the main controller 102 may be provided. A command language between the main controller 102 and a child controller 103 may consist of one or more American Standard Code for Information Interchange (ASCII) characters in some aspects. Firmware running on the child controllers are then programmed to recognize these ASCII character based commands.

FIG. 2B shows an example of a dedicated printed circuit board (shield) 205a for the reward dispenser controller circuit board 103a of FIG. 2A. FIG. 2C shows an example of a dedicated printed circuit board (shield) 205b for the environmental controller 103b of FIG. 2A. In certain embodiments, to prevent errors in the assembly of the child controllers 103a-b, the shields 205a-b were designed with different connectors. For example, shield 205a for the reward dispenser child controller 103a includes a white JST connector 254a while the shield 252b for the physical child controller for the environmental controller 103b includes a Molex connector 254b.

Aspects of the reward dispenser controller circuit board 103a may include one of more of the following functions: dispensing a reward, turning a light on the dispenser on or off, detecting if a dispensing pathway is jammed, or detecting a level of a reward reservoir.

Equipment specific to the role of a particular child controller may connect to the shields 205a-b with additional connectors, for example, Japan Solderless Terminal (JST) or Molex connectors, in some aspects. The ease of attaching or detaching components makes the system easy to maintain. The JST and/or Molex connectors do not require any tools to attach or detach equipment. Furthermore, the connectors are chosen such that it is difficult to plug components in incorrectly. For example, neither the JST nor Molex connector may be connected in a backwards fashion. These connectors provide for system modularity by enabling a variety of devices to be connected to the shields 205a-b. For example, a first product may require a speaker sized for a particular enclosure. A second product may require an enclosure of greater size, or an enclosure to be used in a different environment, such that the size of the speaker needs to be larger. With the use of the above design, the shields 205a-b could remain unchanged for the second product, with a simple modification to the size of the speaker. The connector to the larger speaker would simply plug into the appropriate Molex headers on the existing shield.

The modularity described above provides many advantages. For example, during testing, it was discovered that a first design of a joint tone/sound controller board produced a pause in the white noise whenever a success or failure tone was produced. To solve this problem, the original sound controller was split into two controllers, with a first controller controlling the white noise and a second controller controlling the tones. The modular design of the system enabled this change with only minor changes to the main controller 102. For example, the main controller 102 maintains a list of child controllers and function calls associated with each child controller. The list of function calls available for each separate sound controller was modified to focus on either white noise related functions or tone related functions. After this change was made, the controller was able to interface with each of the separate white noise and tone controllers separately.

Testing protocol may be specified to control testing hardware to conduct an experiment. For example, a testing protocol may first initialize the environment to a known light and sound level. Then, the test protocol may present a stimulus to the subject, record a response, and terminate. As the types of devices available to stimulate the subject and record responses of the subject expand, the possible protocols are limited only by the researcher's imagination and ability to configure the hardware. The features of the present application provide a flexible protocol definition that permit efficient integration of heterogeneous testing hardware to perform experiments with fewer variables than existing systems. Testing hardware may be heterogeneous at a physical level such that two different elements are included for the same purpose. For example, different display devices may be used in two different testing units. Testing hardware may be heterogeneous at an operational level such that two elements of the same physical model may deteriorate in function over time at different rates. For example, an audio speaker included in a first testing unit may not present sound at the same level as an audio speaker included in a second testing unit even though the speakers are identical models.

As an example, one of the experiments that may be used for cognitive testing is generally referred to as concurrent discrimination. Images (referred to as stimuli) may be displayed in pairs. Typically, within each pair, one stimulus is “correct”, while the other is “incorrect.” If the subject selects the correct image, they may be rewarded with a treat and a pleasant tone. If the subject selects the incorrect image or fails to select an image within a specified time limit, they may not receive a treat, and they may hear a less pleasant tone. This process can be then repeated until a desired number of correct answers is achieved. The pairs may be shown in different locations around the screen, or in different orders, but each time a given pair is shown, the same stimulus is “correct.” Ideally, the test subject will learn and remember all of the correct stimuli over several iterations. The more quickly the subject's performance on concurrent discrimination improves, the more likely it is that the treatment administered to the subject was effective.

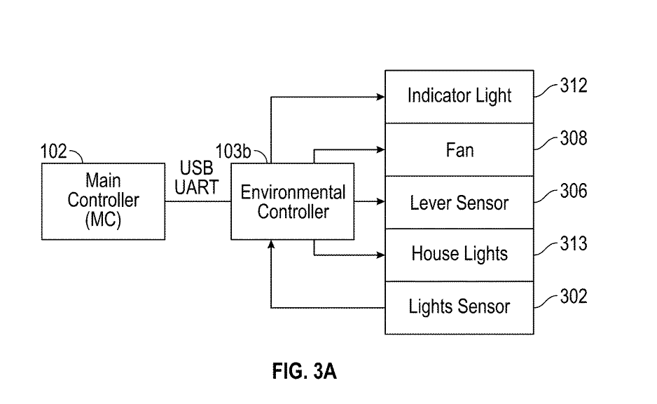

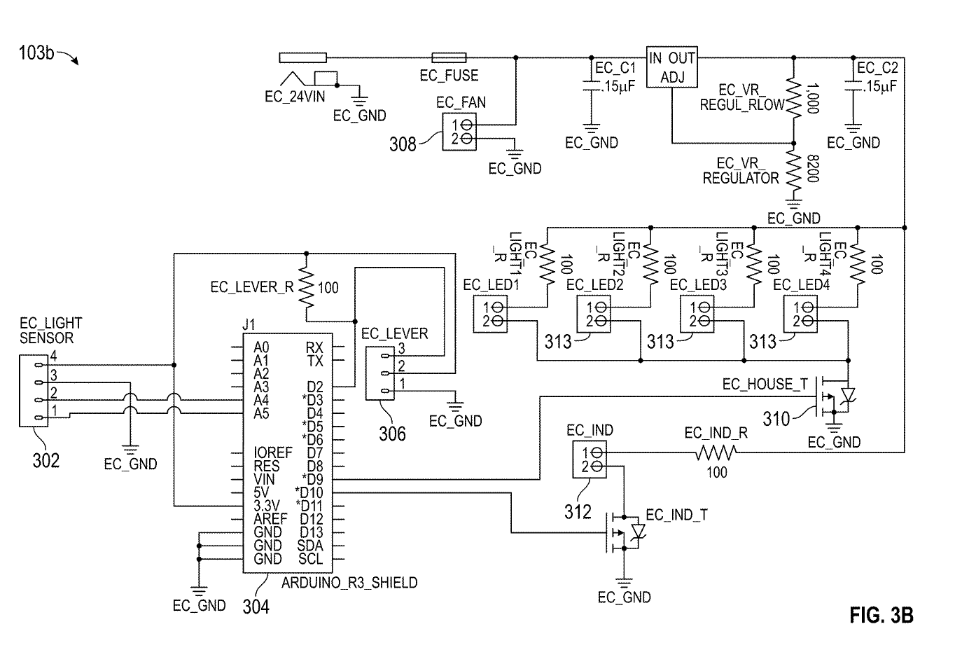

FIG. 3A shows an environment controller 103b coupled to a main controller 102 in one exemplary embodiment. As shown, in some aspects, the environmental controller 103b may also be coupled to one or more devices that either affect the internal environment of the testing chamber or sense a condition of the internal environment. As shown, for example in FIG. 3A, the environment controller 103b is coupled to an indicator light 312, a fan 308, a lever sensor 306, house lights 313, and a light sensor 302. In some embodiments, a temperature sensor, such as a thermometer (not shown) is also included. The temperature sensor may be configured to determine the temperature inside the testing chamber 73. The house lights 313 may include lights configured to provide illumination for testing apparatus environment. For example, where the testing apparatus comprises an enclosure, the house lights 313 may be configured to illuminate the interior of the enclosure.

In certain embodiments, the environment controller 103b may be configured to accept commands form the main controller 102. In certain embodiments, after it receives a command form the main controller 102, the environment controller 103b executes the command and returns a success or failure message to the main controller 102.

In certain embodiments a separate sensor, independent from the environment controller 103b, can confirm the success or failure of the performance of the environment controller 103b. For example, the main controller 102 may instruct the environment controller 103b to turn the lights to a particular brightness level. The light sensor 302 may be configured to determine the actual light level within the testing environment. The light sensor 302 may communicate directly with the environment controller 103b and/or directly with the main controller 102. In some embodiments, the environment controller 103b may confirm the light level by relaying information from the light sensor 302 to the main controller 102. For example, if the house lights 313 are malfunctioning and/or deteriorated over time, when the environment controller 103b commands the light to be at a certain brightness level, the light may not in fact reach the commanded level. As such, the independent light sensor 302 may be used to confirm with the environment controller 103b and/or the main controller 102 that the desired brightness level is in fact reached. In this way, less variability in brightness will occur between tests over time and between subjects. Table 1, below shows exemplary commands, functions, and responses for the main controller 102, environment controller 103b, and sensors 302 and 306.

TABLE 1
Sample Commands and Responses

| | Function | | |
|---|---|---|---|
| Command | performed by | Response by | |
| from main | environment | environment | Sensor |
| controller | controller | controller | Output |
| “a” | Turn off | “indicator | “lux value:” |
| | indicator | light off” | |
| | light | | |
| “b” | Turn on | “indicator | “lux value:” |
| | indicator | light on” | |
| | light | | |
| “I” | Identify | “environment- | N/A |
| | | controller- | |
| | | DEVICE_ID- | |
| | | vCODE_VERSION” | |
| “l” | Set light | “set lux to | “lux value:” |
| | level to | VALUE” | |
| | lux | (success) | |
| | | “lux too low” | |
| | | (dimmed, but | |
| | | not enough) | |
| | | “no headroom” | |
| | | (did not reach | |
| | | max selected | |
| | | value) | |
| (space) | Set light | “set to | “lux value:” |
| | level to | VALUE” | |
| | pulse width | | |
| | modulation | | |
| | (PWM) | | |
| “%” | Set light | “%: VALUE” | “lux value:” |
| | level to | | |
| | percent | | |
| other | Not a | “invalid | N/A |
| | command | input” | |

FIG. 3B is a circuit schematic of one embodiment of the environmental child controller 103b from FIG. 2A. The environmental controller 103b may control non-auditory aspects of the testing environment. The controller 103b includes a light sensor 302, processor 304, in some aspects, an Arduino processor, lever sensor 306, fan 308, house lights 313, and an indicator light 312. In some aspects, the house lights 313 may be dimmable. In some aspects, the lights may be light emitting diodes (LEDs) or another type of light emitting device. In the schematic of FIG. 3B, the house lights operate at 12 V and thus include a transistor circuit 310 to be driven by the processor 304, which outputs 3.3V. The processor 304 generates pulse width modulation (PWM) signals to control the house lights 310. There is a 100 ohm resister as a current limiter for each house light.

The indicator light 312 may be a single light, such as a light emitting diode (LED), and may be positioned on a panel next to the touch screen. The purpose of the indicator light 312 may be to indicate that a testing session is beginning. In the schematic of FIG. 3B, the indicator light 312 may be a 12 volt LED, powered by 12V on the printed circuit board (PCB). A negative-positive-negative (NPN) transistor circuit driven by an Arduino output pin powers the indicator light 312, while a 100 ohm resistor server as a current limiter in the circuit.

The fan 308 may provide for airflow within the testing environment. The fan 308 may also create white noise that is useful in isolating the testing environment from outside noise. In the schematic of FIG. 3B, the fan 308 is directly connected to a 24V input to the environmental controller's PCB. Therefore, the fan 308 is always on when the PCB is connected to 24V, whether the environmental child controller 103b is on or off.

The lever sensor 306 may be in electrical communication with a lever, which may function as an input device. Input received from the lever, via the lever sensor 306, may be in addition to input received from another input device, such as a touch screen. The lever sensor logic of the schematic of FIG. 3B applies a 3.3V signal and a ground (GND) signal to serve as the rails of the lever sensor 306. The output of the lever sensor 306 goes to ground when pressed and is 3.3V otherwise. The Arduino processor of the environmental controller board 103b registers a lever press when the input pin is grounded.

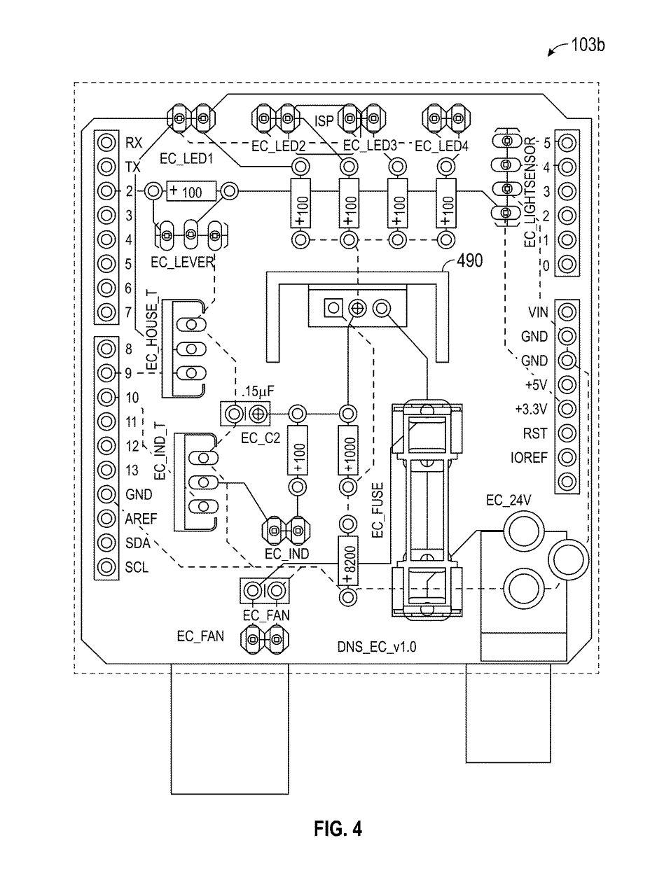

FIG. 4 shows an example printed circuit board (PCB) layout of one embodiment of the environmental controller 103b. The layout shown in FIG. 4 provides adequate spacing of components away from a heat sink 490. In some aspects, the environment controller 103b may receive commands from the main controller 102. The environment controller 103b may perform one or more actions to execute the received command, and then provide a response to the main controller 102, such as a status indication.

### The Reward Dispenser and Controller

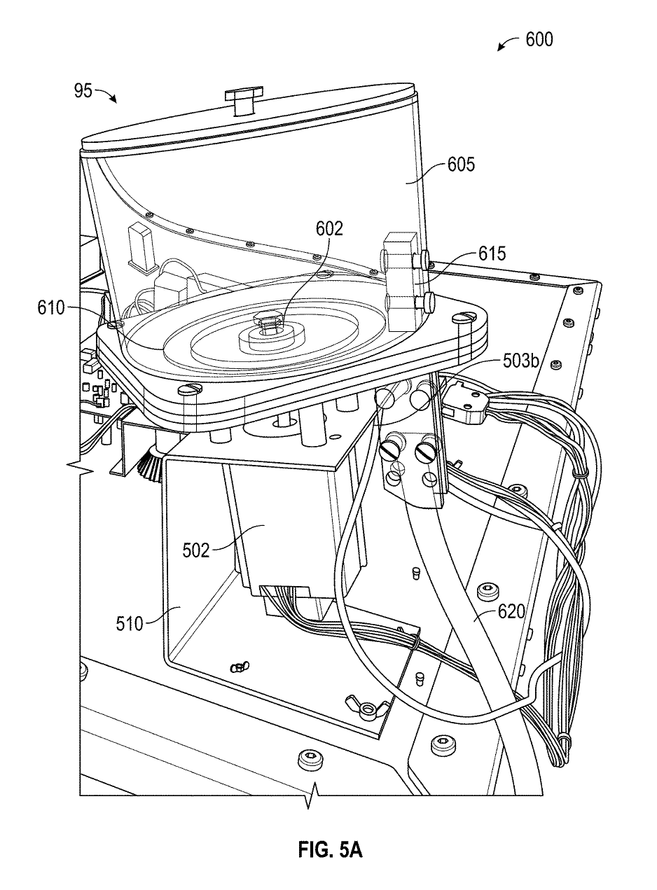

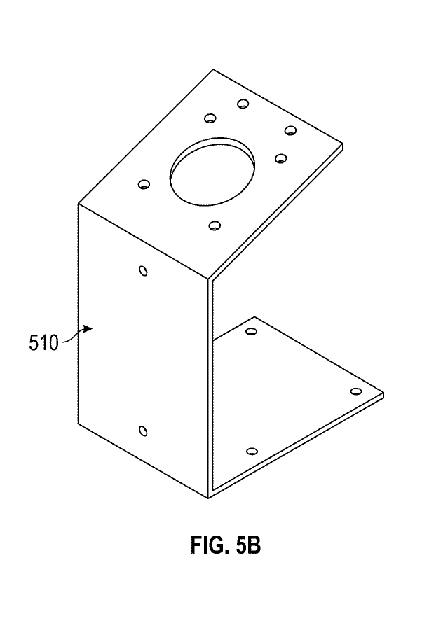

An exemplary reward (food pellet, liquid or paste) dispenser 95 is shown in FIG. 5A. In other embodiments, an edible reward can also include candy or other food items. In some implementations, the reward may be an inedible reward such as a toy, a coin, or printed material (e.g., coupon, sticker, picture, etc.). In some implementations, the reward may be experiential (e.g., song, video, etc.).

As shown, the reward dispenser 95 may include a reward container 605 coupled to a pathway 620. The reward dispenser 95 may be coupled to a mount 510. The mount 510 may be configured to secure the reward dispenser 95 at an angle with respect to the horizontal. It has been found that such an angle may decrease the failure rate of dispensing pellets. Further details of the mount 510 can be seen in FIG. 5B. In some aspects, the mount is configured to secure the reward dispenser 95 at an angle of about ten degrees with respect to horizontal.

Continuing with FIG. 5A, a dispensing plate 602 of the dispenser 95 may include holes 610 that allow the bottom-most pellets to be dispensed first. A limiter 615 may be configured to allow only one pellet to be dispensed at a time. Once dispensed from the container 605, the pellet travels along a pathway 620 to the pick-up area 802 (not shown). In some aspects, the dispensing plate 602 includes grooves configured such that a single pellet fits into each grooves.

In certain embodiments, a motor 502 is configured to step over the width of one hole and a single pellet is dispensed into the pathway 620 containing the dispenser infrared (IR) sensor. Each hole may contain exactly one pellet at any given time so that only one pellet is dispensed from the closest non-empty hole. As shown in FIG. 5A, the limiter 615 may comprise an acrylic block that allows only one pellet to pass by it at a time to ensure that only one pellet is dispensed.

The pathway 620 of a reward dispenser 95 may be monitored by one or more sensors. In some aspects, the sensor is an infrared (IR) sensor 503. As shown in FIG. 5A, a dispenser sensor 503b, is placed so as to observe the pathway 620. When a pellet travels down the pathway 620, it may be sensed by the dispenser sensor 503b. The sensor may be an infrared (IR) sensor 503. A signal may then be sent by the dispenser sensor 503b to a reward dispenser controller board 103a (not shown). The pathway 620 may be coupled to the backside dispenser door shown in FIG. 8 below.

In some aspects, a current sensing device is coupled to the motor 502 and configured to detect larger than normal currents. In this way, potential pellet jams or malfunctions may be detected when the DC motor 502 draws unusually large currents.

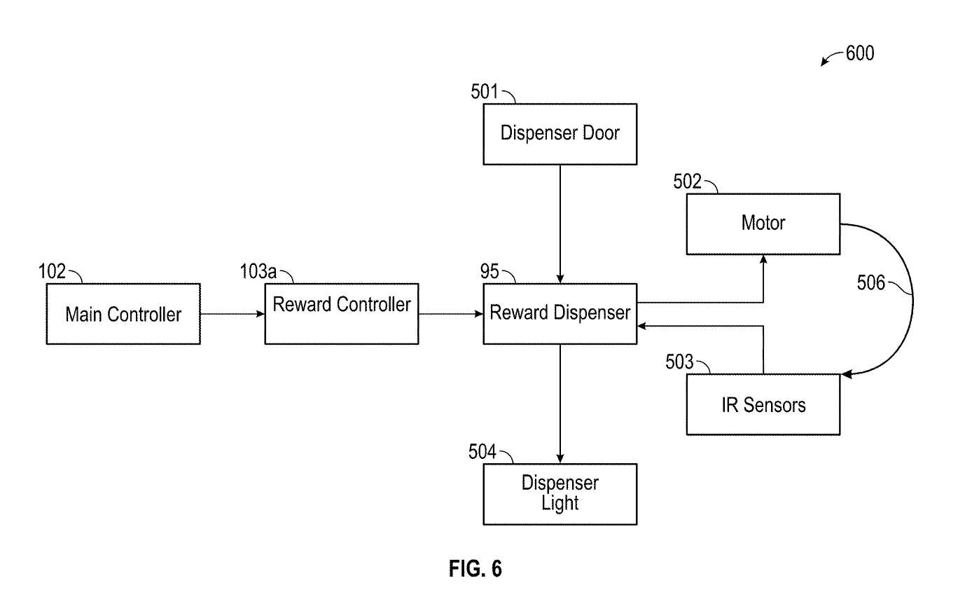

FIG. 6 shows one example of an architecture for a reward dispenser system 600. The reward dispenser system 600 includes the reward dispenser 95, which may provide for a reward to a subject under test. To dispense a reward, the main controller 102 may send a dispense command to the reward dispenser controller board 103a after the main controller 102 detects a correct answer from a subject under test.

In the illustrated embodiment, the architecture 600 includes a dispenser door 601, a stepper motor or motor 602, one or more IR sensors 603, a dispenser light 604, and the reward dispenser 95. The reward dispenser controller 103a is shown directly connected via a USB UART to the main controller 102, discussed previously. The communication flow 606 illustrates that the IR sensors 603 provide feedback regarding the actions of the motor 602.

In certain embodiments, the dispenser system 600 comprises a dispenser door 501, motor 502, a dispenser sensor 503b (shown as part of IR sensors 503 in FIG. 6), a dispenser light 504, and a dispenser door sensor 503a (also shown as part of IR sensor 503 in FIG. 6). In certain embodiments, the motor 502 is configured to drive a revolving mechanism, which dispenses a pellet. For reliable test results, it may be desirable to ensure that a single pellet has successfully been dispensed. If a pellet is not dispensed or if more than one pellet is dispensed, test results may be skewed. As such, one or more sensors may be configured to detect and/or determine if and when a single pellet was in fact dispensed. The sensors may be located at any position in the pellet pathway 620.

The reward dispenser controller board 103a is shown directly connected, in some aspects via a USB UART (not shown), to the main controller 102, discussed previously. The communication flow 506 illustrates that the IR sensors 503 provide feedback regarding the actions of the motor 502. In some aspects the motor 502 may be a stepper motor.

In some cases, the reward dispenser controller 103a commands the dispenser 95 to provide a reward when the main controller 102 sends a dispense command to the reward dispenser controller 103a. This command may be sent, in some aspects, when a subject under test presses a correct image of two or more images that are displayed on a touch screen. If the subject touches the incorrect image, no command is given to the reward dispenser controller 103a and no pellet is dispensed, at least in some aspects. However, the system can dispense a reward whenever the main controller 102 instructs the reward dispenser 95 to dispense a reward.

In certain embodiments, the reward dispenser controller 103a is configured to notify the main controller 102 when a reward is jammed in the dispensing pathway 620 of FIG. 5A or when there are no more rewards to dispense, as when the container 605 is empty. As such, one or more sensors may be positioned in the container 605, dispensing pathway 620, and/or at or near the exit or entrance of the dispensing pathway 620. In some aspects, the sensor is an infrared sensor.

In certain embodiments, the exit to the reward dispenser pathway 620 includes a door 501 that must be opened by the test subject in order to retrieve the reward. Thus, in some aspects, the reward dispenser system 600 includes a light (e.g., an LED light) 504 attached to or adjacent to the reward dispenser door 501. This light 504 may be configured to notify the test subject that it can pick-up its reward through the dispenser door 501. After the test subject opens the dispenser door 501 and retrieves the reward, another sensor 504 may be configured to recognize that the door 501 has opened and the light 504 is then turned off by the reward dispenser controller 103a.

Accordingly, in certain embodiments the reward dispenser controller 103a is configured to do one or more of the following: dispense a reward via the dispenser 95, control a dispenser light 504, detect jams in the dispensing pathway 620 via an IR sensor 503b, determine when there are no more rewards to dispense from the container 605, confirm that only one reward was dispensed, and communicate with the main controller 102. Table 2, below shows exemplary commands, functions, and responses for the main controller 102, reward dispenser 95, and/or one or more sensors 503.

TABLE 2
Example main controller commands
recognized by reward controller

| Command | | |
|---|---|---|
| from main | Function performed | Response by reward controller |
| controller | by reward controller | and/or sensor |
| “p” | Dispense a pellet | See Table 3 (below) |
| “i” | Identify | “pellet-dispenser- |
| | | DEVICE_IDvCODE_VER- |
| | | SION” |
| “other” | Not a command | “invalid input” |

Possible responses from the reward dispenser controller 103a are also shown in Table 3.

TABLE 3
Possible responses from the reward controller after “p” command.

| Control Result | Response |
|---|---|
| Successful | “pellet dispensed” |
| Fail to dispense after the motor's third step | “reached timeout” |
| Detect a jammed pellet | “dispenser jammed” |

In some aspects, a current sensing device is coupled to the motor 502 and configured to detect larger than normal currents. In this way, potential pellet jams or malfunctions may be detected when the motor 502, which may be a direct current (DC) motor in some aspects, draws unusually large currents.

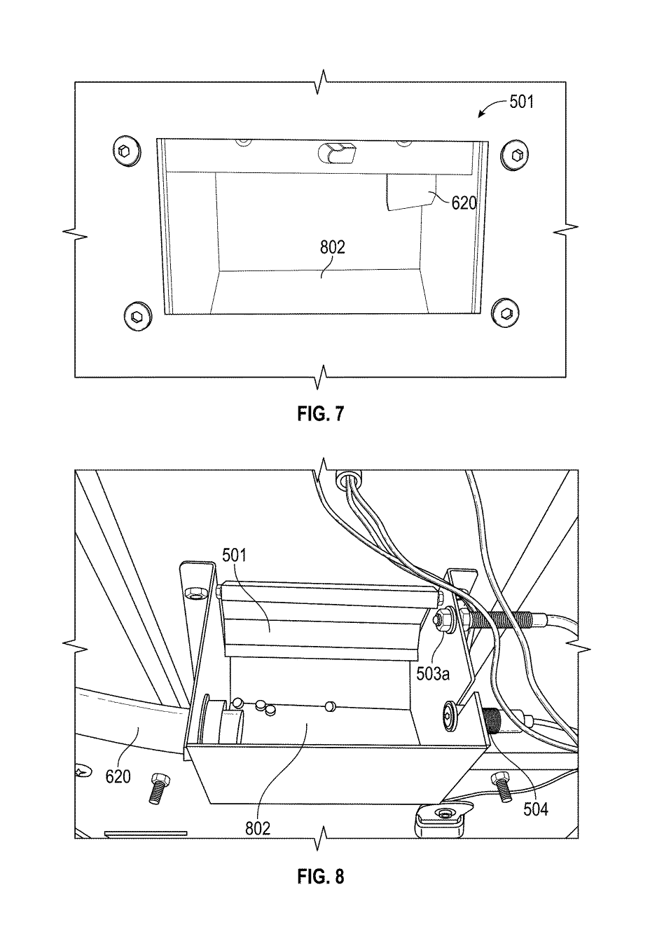

FIG. 7 shows a front view of the dispenser door 501. A pick-up area 802 behind the dispenser door 501 is also shown.

FIG. 8 shows a pick-up area 802 behind the door 501. The pick-up area 802 may include a dispense light 504 and a pickup door IR sensor 503a. The dispense light 504 may be configured to illuminate when the dispense IR sensor 503b of FIG. 5A detects a pellet has exited the pathway 620. In some aspects, the dispense light 504 may be a white, 3.3 V LED. The dispense light 504 may be configured to be turned off when the dispenser door IR sensor 503a detects movement of the dispenser door 501. The pick-up area 802 may be accessible from within the testing chamber 73, shown in FIG. 1A. For example, after a reward is dispensed to the pick-up area 802, an animal under test may access the pick-up area 802 to retrieve the reward.

In some aspects, the dispenser door sensor 503a includes one or more diffuse IR sensors that take in two inputs of 24 volts and GND and have a single output of high or low. In some aspects, when a pellet passes through the diffuse beam, the IR light is reflected back to the transmitter and receiver end and the sensor outputs HIGH, or 24 V. In some aspects, a voltage divider steps this down to five volts, as an Arduino processor in a child controller circuit board 103a controlling the dispenser system 600 may not be able to handle voltages above this value.

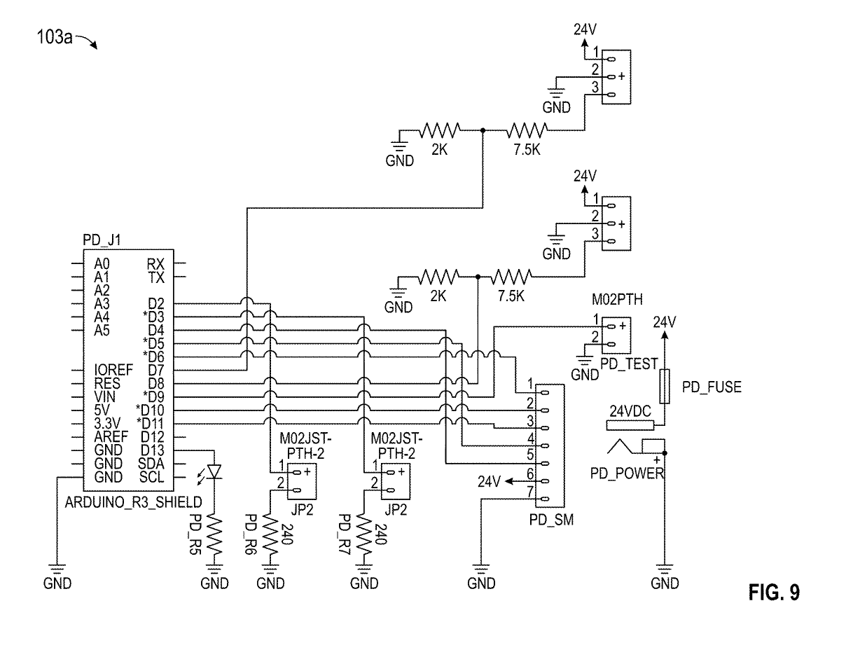

FIG. 9 shows a schematic of a printed circuit board for the reward dispenser controller board 103a. The schematic illustrates various components including a processor, in some aspects, an Arduino processor, resistors, and the like. In certain embodiments, the processor generates signals to control the reward dispenser 95.

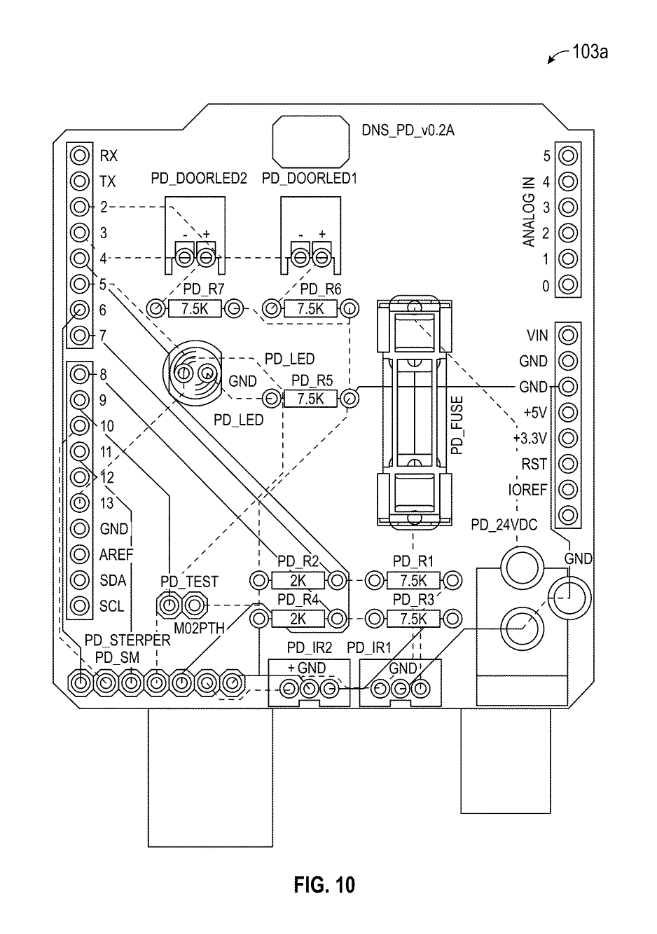

FIG. 10 shows an example layout of the printed circuit board for the reward dispenser controller board 103a.

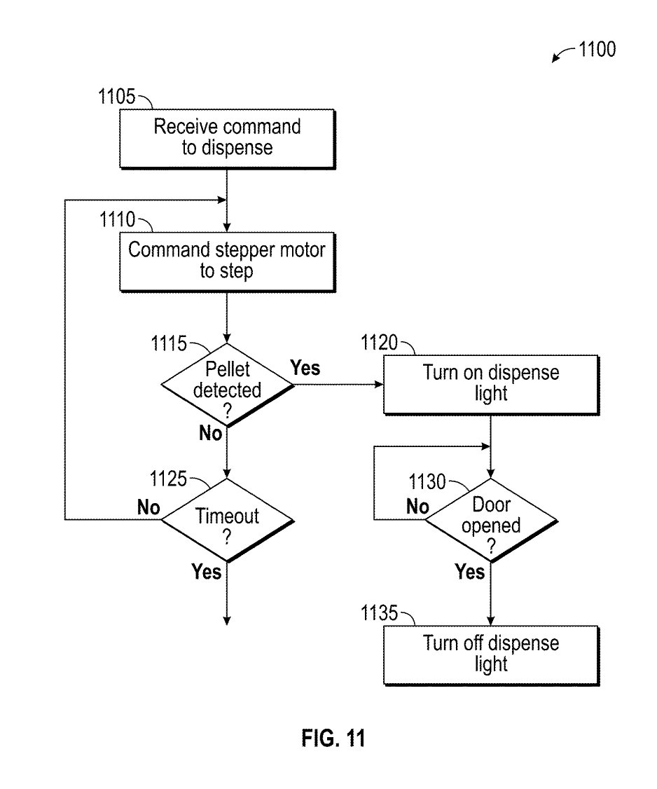

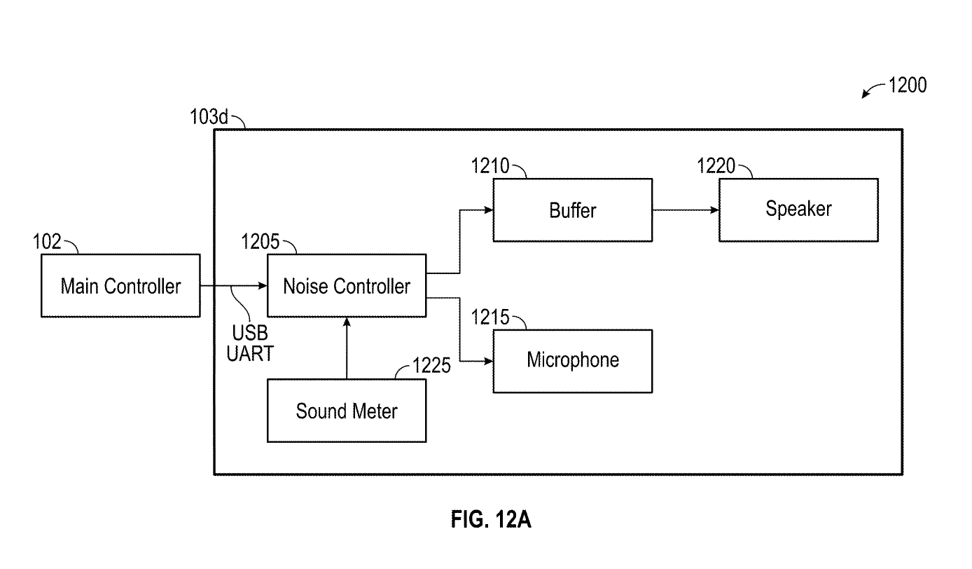

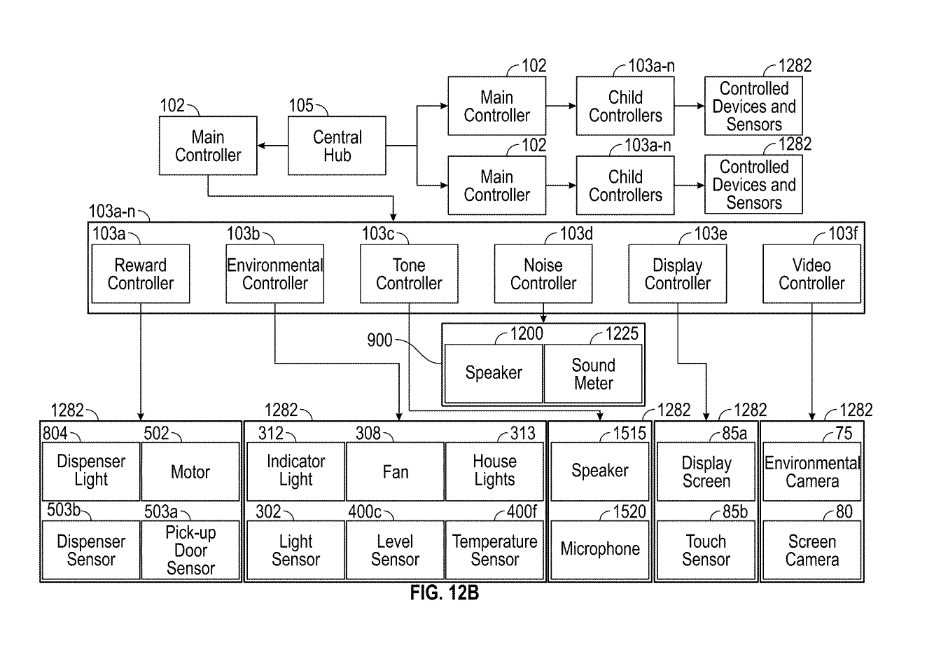

FIG. 11 is an example method for dispensing a pellet utilizing the reward dispenser architecture shown above with respect to FIG. 6. In some aspects, the method 1100 may be performed by a reward dispenser controller board, such as the board 103a shown in FIG. 2A or the board 103a shown in FIG. 9. In block 1105, a command is received to dispense. In some aspects, the reward dispenser 95 dispenses a pellet. In some aspects, the command may be received from the main controller 102, discussed above. In some aspects, the command may be received over a serial I/O bus, such as a USB bus.

In block 1110, a stepper motor 502 is commanded to move by a specified angle (corresponding to one or more steps). In some aspects, the stepper motor 502 may be configured such that the move angle that corresponds to a width of a groove in a dispensing plate, for the example, the dispensing plate 602 of FIG. 5A. Decision block 1115 determines whether a pellet has been detected. In some aspects, detection of a pellet may be performed by reading output from an IR sensor, such as the IR sensor(s) 503a-b shown in FIGS. 5A, 6 and 8. In block 1110, a stepper motor 502 is commanded to step. In some aspects, the stepper motor 502 may be configured such that a single step corresponds to a width of a groove in a dispensing plate. Decision block 1115 determines whether a pellet has been detected. In some aspects, detection of a pellet may be performed by reading output from an IR sensor, such as the IR sensor(s) 503 shown in FIG. 6. If a pellet is not detected, block 1125 determines if a timeout has occurred. In some aspects, a timeout may be detected if the number of commands sent to the motor 502 in block 1110 in a particular dispense cycle is above a threshold. In some aspects, the threshold may be 2, 3, 4, 5, 5, 7, 8, 9, or 10 steps of the stepper motor. If no timeout has occurred, processing returns to block 1110 where the motor 502 is commanded to move again. If a timeout is detected in decision block 1125, processing continues. In some aspects, an error condition may be raised to an operator for example.

If a pellet is detected in block 1115, a dispense light, such as dispense light 504 of FIG. 6 is turned on in block 1120. The dispense light may be positioned to provide a visual signal to the subject upon dispensation of a pellet. For example, the dispense light 504 may be positioned within proximity to a dispenser door 501, as shown in FIGS. 7-8. Decision block 1130 determines whether a reward dispenser door 501, such as shown in FIGS. 7-8, has been opened. The detection of the door 501 being opened may be based on input from an IR sensor, such as IR sensor 503a shown in FIG. 8. If the door 501 has not been opened, the process returns to block 1130 to see if the door 501 has been opened. If the door 501 has been opened, process 1100 moves to block 1135, where the dispense light 504 is turned off. Processing then continues.

FIG. 12A illustrates an example architecture for use with the noise controller circuit board 103d. The noise controller circuit board 103d of FIG. 12A may adjust an enclosure's noise level to be within a DB range sound pressure level (SPL) value. When a test begins, an initial noise level for the enclosure may be specified as a test parameter. The controller 103d may set the noise level to be within the DB range of the specified noise level.

Architecture 1200 shows a noise controller 1205, buffer 1210, microphone 1215, speaker or tone emitter 1220, and a sound meter 1225. In the illustrated aspect, the noise controller 1205 may be a hardware chip on the noise controller circuit board 103d. For example, in some aspects, the noise controller 1205 may be an Arduino processor as shown. However, other embodiments may utilize different controller hardware. The noise controller 1205 is in communication with the main controller 102, discussed previously. In some aspects, the noise controller 1205 and the main controller 102 communicate using a Universal Serial Bus (USB), as shown.

In the architecture 1200, the noise controller 1205 may receive commands from the main controller 102. For example, the commands may be received over a bus, such as a USB bus. The noise controller 1205 may be configured to perform the commanded task and provide a result indication to the main controller 102 after the commanded task has been completed. The noise controller 1205 may read audio data from the microphone 1215. The noise controller 1205 may output tone signals to a buffer 1210, which then provides the signals to a speaker 1220.

As shown, the noise controller 1205 is also coupled to a sound meter 1225. The sound meter 1225 may be configured to determine the level of ambient noise within the enclosure.

As discussed above, the tone controller 103c may be configured to generate success or failure tones when the test subject completes a task. These tones may serve as an extension of the rewards system. The tone controller 103c may be responsible for playing a success tone when the subject correctly answers a question and/or a failure tone when the subject answers incorrectly. The tone controller 103c must be loud enough for the test subject to hear the tone over the sound played by the noise controller 103d. The tone controller 103c may be configured to play a tone of desired frequency, duration, and volume and able to produce identical tones throughout the experiment. The tone controller 103c may be configured to play success or failure sounds at different frequencies, sound levels, and durations. However, it is suggested that the researchers specify a particular volume level and duration for both tones and choose one specific frequency for each the success and failure sound. In some aspects, this may reduce variability in test results.

FIG. 12B illustrates an exemplary embodiment of the modular architecture discussed above. In some aspects, the child controllers 103a-n and the controlled devices/sensors 1282 shown in FIG. 12B may be organized as shown in the examples of FIG. 12A where applicable. As shown, a central hub 105 may be configured to control a plurality of main controllers 102. The main controllers 102 can each be configured to control a plurality of secondary controllers 103a-n as described above.

In some embodiments, a script, running on the central hub 105 initiates a command. The command is in turn sent to a respective main controller 102. The main controller 102 then looks up the command from a configuration table to find the corresponding command(s) to send to on the correct child controller 103a-n. The correct child controller 103a-n then executes the command(s) received from the main controller 102. That is to say, the child controller 103a-n instructs the respective hardware devices/sensors 1282 to execute the command (e.g. dispense a pellet, set an internal temperature, display one or more images, etc.). An associated sensor may be used to confirm that the hardware device in fact executed the command(s). The associated sensor may send a signal to the corresponding child controller 103a-n confirming that actions were in fact taken. The child controller 103a-n can in turn send a complete command to the main controller 102 that can be forwarded on the central hub 105. The central hub 105 can then trigger an event in the script and the script can record the event.

In some embodiments, a child controller 103a-n can initiate an event. For example, the touch sensor 185b may record a touch event on the display screen 185a and forward this information to the display controller 103e. The display controller 103e may then forward this information to the main controller 102 which can trigger a corresponding event according to the particular testing routine. For example, a correct touch may cause the display controller 103e to signal the main controller 102 to send a dispense command to the reward controller 103a. A correct touch may also cause the main controller 102 to send a correct touch signal to the central hub 105 such that a script running on the central hub 105 can record the event.

In some embodiments, more than one script can be run concurrently such that multiple subjects may be tested at once. That is to say, the central hub 105 may be able to control and monitor tests occurring in different testing enclosures at the same time. A user may initiate a script for a first subject in a first system and a second script for a second subject in a second system. The central hub 105 executes the script and saves the results to a local event log. The main controller 102 handles the requests and events. The child controllers 103a-n communicate with the dedicated hardware and sensors associated with the respective child controller 103a-n.

In some embodiments, the system may be calibrated prior to a test being run. The central hub 105 may send a calibrate command to the main controller 102. The main controller 102 can then commend the child controller(s) 103a-g to begin calibration routines and interact with the sensors to confirm that the system is calibrated. For example, the main controller 102 can command the environmental controller 103b to set the lighting to a particular level. The main controller 102 and/or environmental controller 103b may in turn request a light level measurement from the light sensor 302. This may be repeated until the desired light reading is measured by the light sensor 302. In another example, the main controller 102 can command the noise controller 103d to set the white noise to a particular level via the speaker 1220. The main controller 102 and/or noise controller 103d may in turn request a sound level measurement from the sound meter 1225. This may be repeated until the desired sound meter 1225 reading is measured.

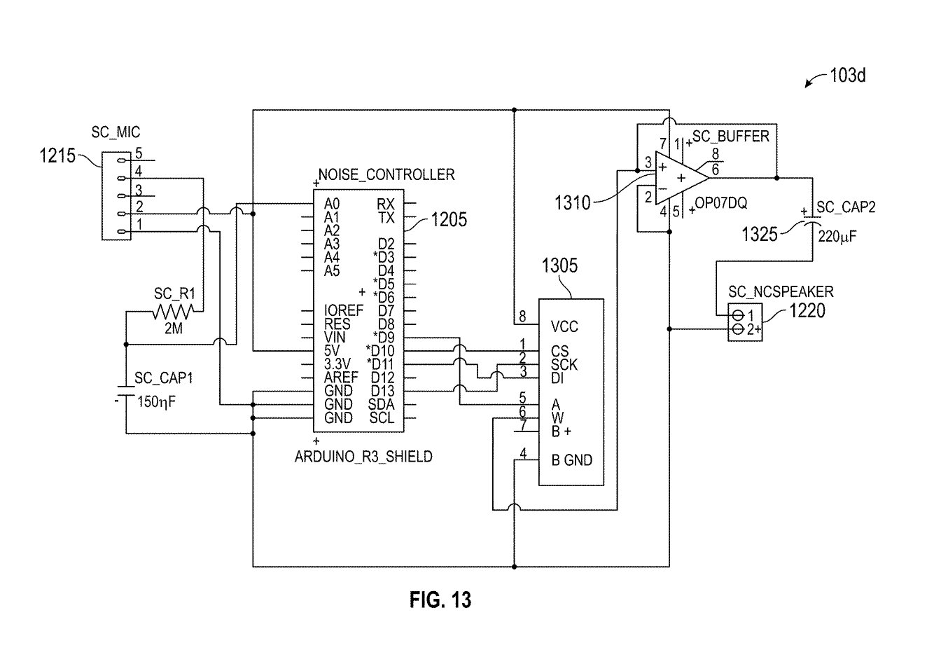

FIG. 13 shows an example schematic for the noise controller 103d. In some aspects, the noise controller 103d may include one or more of a digital potentiometer 1305, amplifier 1310, speaker 1220, and microphone 1215. The digital potentiometer 1305 is configured to provide volume control for the noise controller 103d. The digital potentiometer 1305 is configured as a voltage divider for an audio signal. The noise controller 1205 is configured to set a resistance value of the potentiometer 1305 via a Serial Peripheral Interface (SPI). The amplifier 1310 is configured as a unity gain buffer for the speaker 1220. The amplifier 1310 isolates the speaker 1220 from other hardware to prevent effects from interference from the remainder of the circuit. The speaker 1220 plays sound corresponding to a received voltage signal. The schematic of FIG. 13 shows a decoupling capacitor 1325 of 220 uF to remove a DC offset before the signal goes to the speaker 1220.

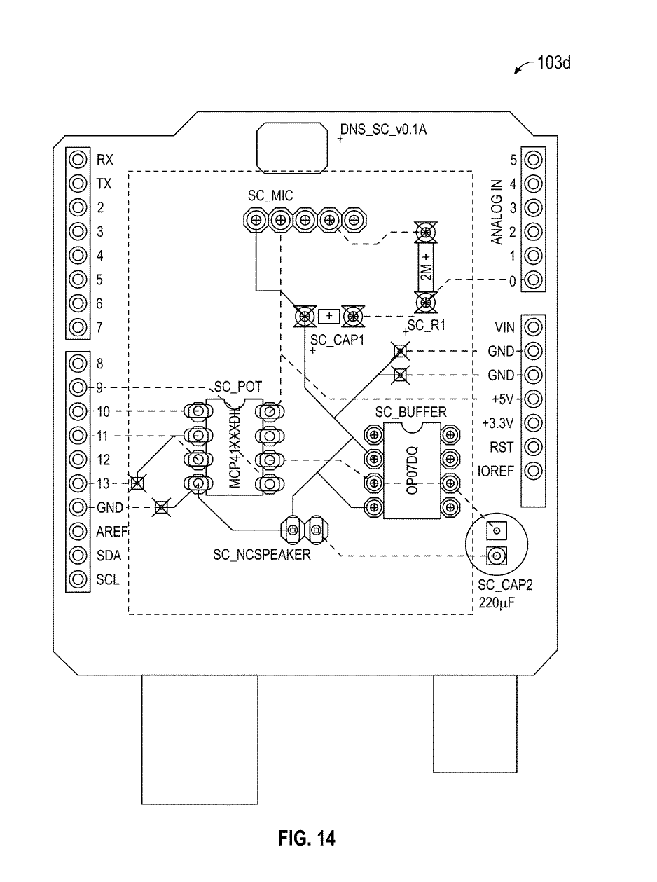

FIG. 14 shows an example printed circuit board layout for a noise controller 103d.

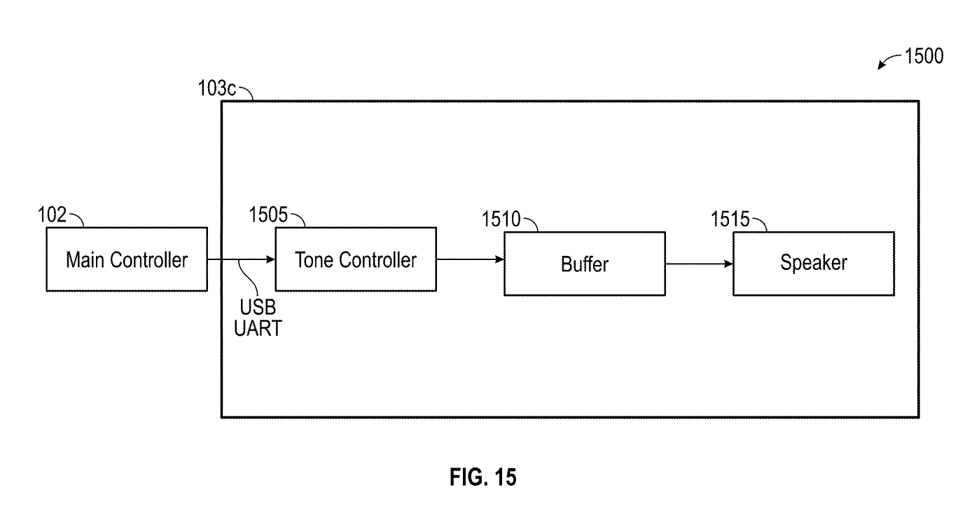

FIG. 15 shows an example architecture 1500 for a tone controller circuit board 103c. The architecture 1500 includes a tone controller 1505, buffer 1510, and speaker 1515. In some aspects, the tone controller 1505 may be a hardware chip that is part of the tone controller circuit board 103. For example, the tone controller 1505 may be an Arduino processor. The tone controller 1505 may be in communication with a main controller 102. In some aspects, the communication between the main controller 102 and the tone controller 1505 may be performed over a serial bus, such as a universal serial bus. The main controller 102 may send commands to the tone controller 1505. The tone controller 1505 may be configured to process the command. Processing the command may include generating signals over a bus on the tone controller circuit board 103c so as to cause the speaker to generate a tone, and/or to read audio signals from a microphone 1215. Processing the command may also include reading a sound level from the sound meter 1225. After processing of the command is completed, the tone controller 1505 may send a response, for example, over the bus, to the main controller 102. In some aspects, the response may indicate a completion status of the command. The tone controller 1505 may output data defining audio signals to the buffer 1510, which sends the signals to the speaker or tone emitter 1515.

In some aspects, the system may also include a weight controller (not shown in FIG. 15). The weight controller may comprise a scale that is internal to the system. The weight controller may be coupled to the main controller. In certain embodiments, a cognitive test may query the weight of the test subject before and/or after the cognitive test.

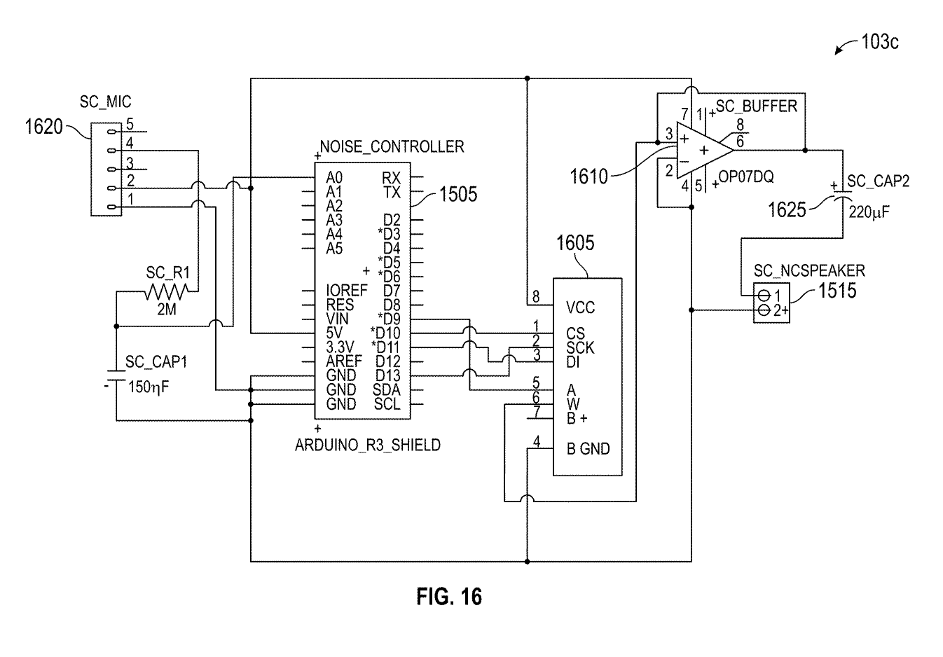

FIG. 16 shows an example schematic for the child controller 103c from FIG. 15. In some aspects, the tone controller 103c may include one or more of a noise controller 1505, digital potentiometer 1605, amplifier 1610, speaker 1515, sound detector 1620, and capacitor 1625. The digital potentiometer 1605 is configured to provide volume control for the noise controller 1505. The digital potentiometer is configured as a voltage divider for an audio signal. The noise controller 1505 is configured to set a resistance value of the potentiometer 1605 via an SPI interface. The amplifier 1610 is configured as a unity gain buffer for the speaker 1315. The amplifier 1610 isolates the speaker 1515 from other hardware to prevent effects from interference from the remainder of the circuit. The speaker 1515 plays sound corresponding to a received voltage signal. The schematic of FIG. 16 shows a decoupling capacitor 1625 of 220 uF to remove a DC offset before the signal goes to the speaker 1515. In some aspects, the tone controller board 103c may utilize the same hardware as the noise controller 103d. In some of these aspects, the tone controller may not include the microphone 1215 that is included on the noise controller 103d. In these aspects, the main controller 102 may be configured to use the microphone 1215 installed on the noise controller board 103d to calibrate a tone volume generated by the tone controller board 103c.

FIG. 17A shows an example printed circuit board layout for a tone controller 103c. As discussed above, in some aspects, the tone controller 103c may receive commands from the main controller 102. The tone controller 103c may perform one or more actions to execute the received command, and then provide a response to the main controller 102. The response may include information such as a status of execution of the command. such as a status indication.

FIG. 17B shows an example printed circuit board layout for a noise controller, such as the noise controller 103d.

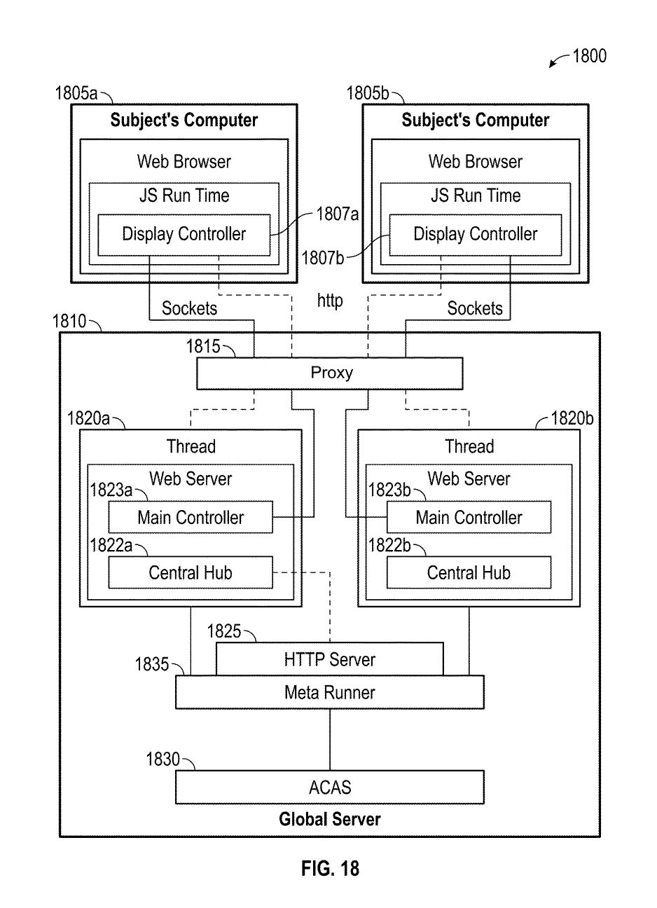

FIG. 18 shows an example system configuration 1800 for electronically controlled animal testing. The system configuration 1800 includes two test stations, each test station including a computer 1805a-b. Each computer 1805a-b includes a web browser, such as Firefox, Chrome, or Internet Explorer, a JavaScript runtime executing inside the browser, and a display controller 1807a-b, which in some aspects may be a set of instructions implemented in a JavaScript program. In some aspects, each of the display controllers 1807a-b may control separate electronic displays positioned within corresponding animal test enclosures, such as animal test enclosure 50 illustrated in FIG. 1A. In some implementations, a computer may include a thick client or other software specially configured to present a user interface.

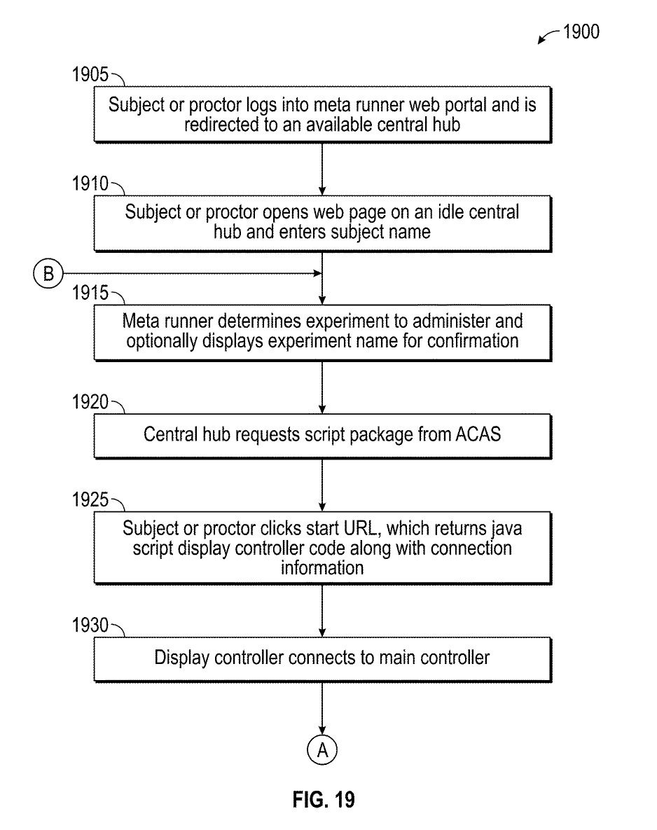

Also included in the configuration 1800 is a global server 1810. The global server 1810 may include a hardware processor and hardware memory (not shown) storing instructions that configure the hardware processor to perform one or more functions discussed below with respect to the global server. The global server 1810 includes a proxy 1815, which may be implemented using Nginx in some aspects, two web server execution thread processes 1820a-b, an http server 1825, and Assay Capture and Analysis System (ACAS) 1830. Running on the http server 1825 is a scripted meta runner 1835. In some embodiments the meta runner performs the functions of the meta hub. In some embodiments, ACAS 1830 is used as a meta hub 132. The webserver processors 1820a-b include central hubs 1822a-b and main controllers 1823a-b respectively. The web server processors 1820a-b may be implemented using multiple instances of a python web server and runtime environment like Tornado commercially provided by The Tornado Authors. The central hubs 1822a-b run inside the web server. The meta runner 1835 may be a protocol test configuration runner such as a test script runner. Although only two threads are shown, the system may be architected to support 100, 1000, 10000, or more threads. FIG. 19 is a flowchart of a method that may be performed using the configuration 1800 of

FIG. 18. In block 1905, a subject or proctor logs into a meta runner 1835 and is redirected to an available central hub, such as central hubs 1822a-b. In some aspects, each of the meta runner and/or central hub may be physically separate electronic hardware computers, each comprising a hardware processor and hardware memory. Each of the hardware memories may store instructions that configure each of the respective hardware processors to perform functions discussed below attributed to the meta runner and central hub respectively.

In block 1910, a subject or proctor opens a web page on an idle central hub and enters the subject name (or has it entered for them). Other attributes of the test subject may also be received in block 1910. For example, in some aspects, one or more of a subject identifier number, a subject weight, a subject sex, and a subject species may be received. In some aspects, after the subject attributes are entered, they may be stored to a data store, such as a laboratory information system that provides centralized access to subject information.

In block 1915, the meta runner 1835 determines a test or experiment to administer and optionally displays the test or experiment name for confirmation. In some aspects, the meta runner 1835 may determine the test or experiment to administer by interfacing with a meta hub, such as meta hub 132 shown in FIG. 1E.

In block 1920, a central hub (such as one of central hubs 1822a-b) requests a script package from ACAS 1830. In block 1925, a subject or proctor clicks a start URL, which returns java script display controller code (instructions) along with connection information. In block 1930 the display controller connects to a main controller, such as one of main controllers 1823a-b running with either of the subject computers 1805a-b respectively. Block 1930 may include receiving a connection request from the display controller at the main controller. Block 1930 may also include administering, by the first computer, the experiment by sending and receiving data over the connection after the connection request is established.

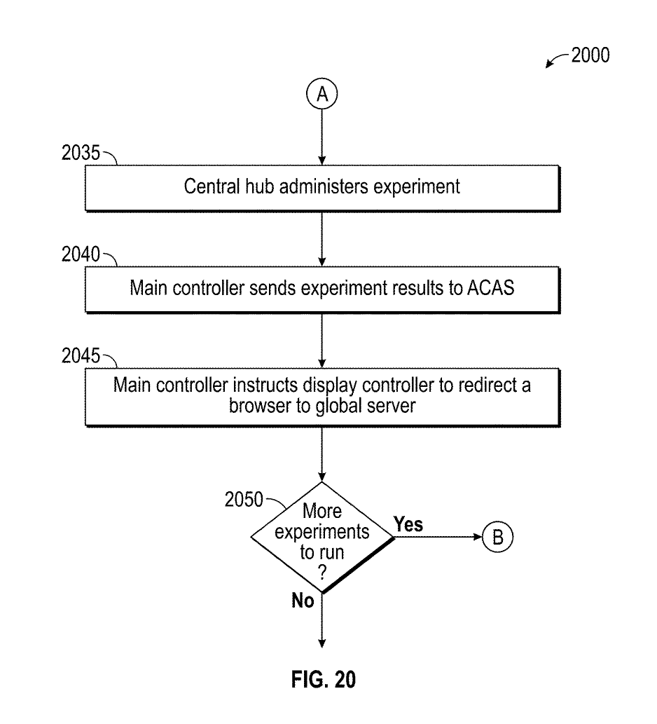

Transitioning to an exemplary method 2000 FIG. 20 through off page reference “A”, in block 2035, a central hub administers the test. In block 2040, the main controller sends test results back to ACAS 1830. In block 2045, the main controller instructs the display controller to redirect the browser to the global server 1810. Decision block 2050 determines whether additional tests are available for running. If not, process 2000 continues processing, below. If more tests are available, process 2000 moves through off-page reference “B” to block 1915 in FIG. 19 and processing continues.

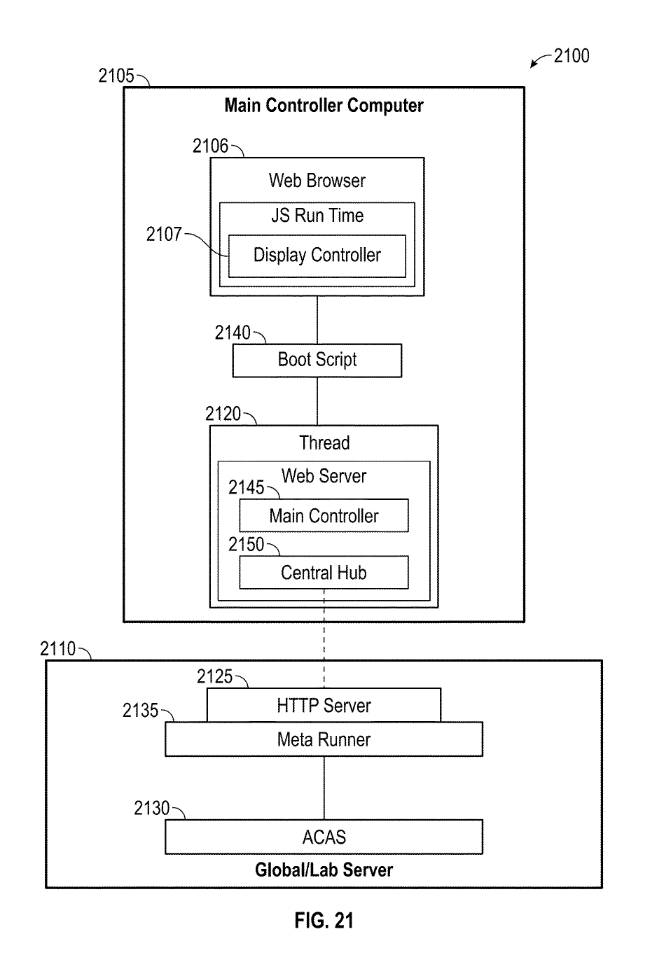

FIG. 21 is a block diagram of a system configuration 2100 including a main controller computer 2105 and a global/lab server 2110. The main controller computer 2105 includes a web browser 2106, which includes a java script run time environment, and a display controller 2107, and a boot script 2140, a hardware processor and hardware memory (neither of which are shown) storing instructions that configure the hardware processor to perform functions discussed below performed by the main controller. Executing on the main controller computer are a web browser 2106, which includes a JavaScript run time environment, and a display controller 2107. A webserver 2120 executing on the main controller computer 2105 includes a main controller 2145 and a central hub 2150. The thread 2120 includes a web server, a main controller 2145, and a central hub 2150. The Global/lab server 2110 is a hardware computer including a second hardware processor and a second hardware memory storing instructions that configure the hardware processor to perform one or more of the functions discussed below with respect to the global/lab server. For example, the instructions may cause the second hardware processor to implement an http server 2125, meta runner 2135, and ACAS 2130.

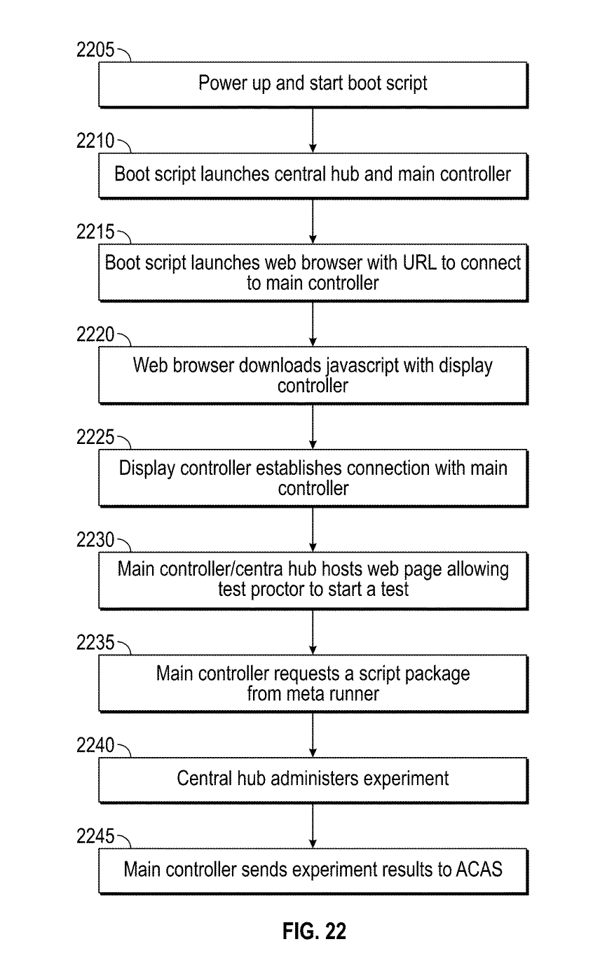

FIG. 22 is a flowchart of a method that may be performed using the system configuration 2100 of FIG. 21. In block 2205, power up of the main controller computer 2105 occurs and the boot script 2140 starts. In block 2210, the boot script 2140 launches the central hub 2150 and main controller 2145. Launching the central hub 2150 and main controller 2145 may include instantiating each of the central hub 2150 and main controller 2145. In block 2215, the boot script 2140 launches the web browser 2106 with a uniform reference locator (URL) to connect to the main controller 2145. In block 2220, the web browser 2106 downloads JavaScript implementing the display controller 2107. In block 2225, the display controller 2107 establishes a connection with the main controller 2145. In some aspects, the connection may be made via web sockets. In block 2230, the webserver 2120 hosts a web page allowing a test proctor to start a test. In block 2235, the main controller 2145 requests a script package from the meta runner 2135. The script package may be considered experiment control information or test control information in some aspects. In block 2240, the central hub 2150 administers the test. In some aspects, the test may be administered between the central hub and the main controller via the established communication or connection from block 2225. Administration of the test in block 2240 generates test results. In block 2245, the main controller 2145 sends the test results to ACAS.

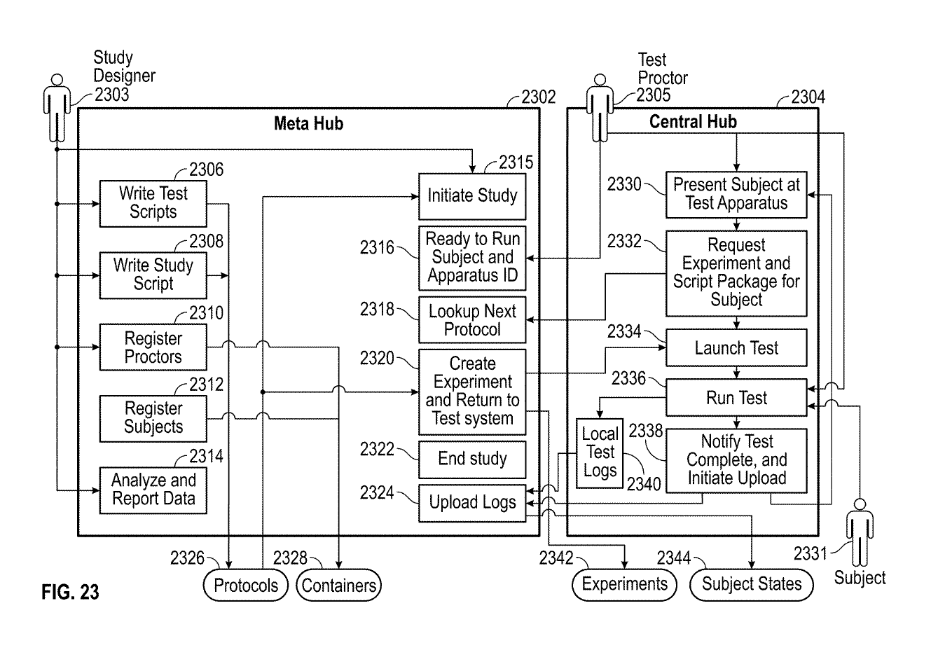

FIG. 23 is a data flow diagram of a study design and test process. An experiment as used in this context is a single test with a single subject as implemented by the system 101 as described above. A study, also referenced in the discussion of FIG. 1E above, is a set of experiments with multiple subjects and/or multiple experiments per subject. The study defines the set of individual tests required, for example, to measure how fast an individual subject learns over multiple test sessions, and/or how a group of subjects who have received treatment compare to a second group of subjects that have not been treated. A protocol, referenced below, is a predefined recorded procedural method used in the design and implementation of the experiments.

The study design is managed by a global runner meta hub 2302. A study designer 2303 writes test scripts 2306, writes study scripts 2308, registers proctors 2310, registers subjects 2312, and analyzes and reports on data 2314. These processes generate protocols 2326 and containers 2328. The protocols 2326 are used to create experiments in block 2320, which are stored in an experiments data store 2342.

The study designer 2303 may then initiate a study 2315, which also relies on the protocols 2326. As part of the study, a test proctor 2305 presents a subject 2331 at a test apparatus 2330, and requests 2332 an experiment and script package for the subject 2331 (block 2318). This causes a request 2332 to be generated from the test system 2304 to lookup the next protocol 2318 via the global runner meta hub 2302. The meta hub 2302 The global runner may then create an experiment 2320 record as a place to store test script results and retrieve the test script from the experiment data store 2342 and return it to the test system 2304 so that the test can be launched in block 2334. After the test is run in block 2336, test logs 2340 are created. A notification that the test is complete is performed in block 2338 and an upload of the test logs 2340 may be initiated via block 2324 of the global runner 2302, after the study ends 2322. meta hub 2302.

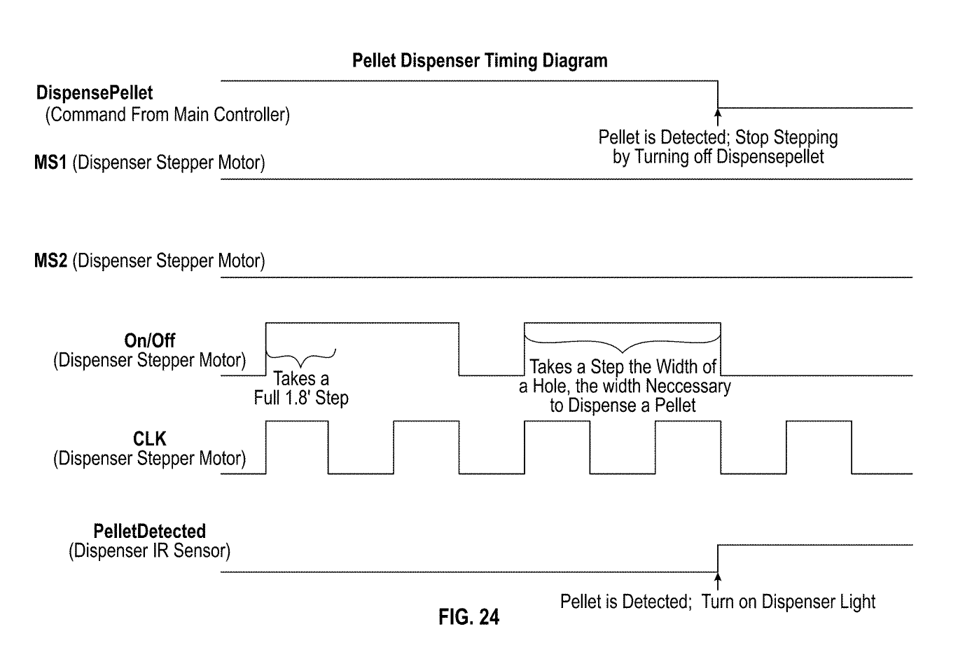

FIG. 24 is an exemplary timing diagram for a reward dispenser, such as the reward dispenser 95. In some embodiments, the motor 502 may be a stepper motor. As shown, in certain embodiments, the main controller 102 can instruct the reward controller 103a to dispense a reward, such as a food reward. In turn, the reward controller 103a can instruct the motor 502 to rotate. In some aspects, when the one or more sensors 503 detects the dispensing of a reward, the motor 502 may be stopped and a dispenser light 504 may be turned on. The reward dispenser 95 and/or reward controller 103a can then confirm to the main controller 102 that the reward was dispensed and the cognitive test may continue.

In some aspects, the motor 502 takes two voltage inputs of 24 volts and ground (0 volts). The dispenser motor 502 may take additional input signals, CLK, ON/OFF, MS1, and/or MS2. The CLK signal provides the clock signal for the motor from the Arduino. When the Arduino drives the ON/OFF signal HIGH, the motor 502 turns one step. Lastly, the MS1 and MS2 signals may control the width of every step. In certain embodiments, when both MS1 and MS2 are set to LOW, the motor 502 is configured to step of 1.8 degrees.

In certain embodiments, the reward controller 103a is configured to receive a single command to dispense a reward from the main controller 102. When the main controller 102 sends the command to dispense a reward, the reward controller 103a starts by telling the motor 502 to step continuously until a reward is detected or a timeout is reached. In some aspects, the timeout is set to 3 steps, which means the reward controller 103a will stop if the dispenser sensor 503 does not detect a reward by the end of the motor's 502 third step. The motor 502 may be configured to turn for the width of each groove in the dispensing plate 602 of the pellet dispenser 95. After each step, the reward controller 103a may check the reward detection status. The value representing this status turns on when a reward has been dispensed, which is triggered by an analog interrupt continually checking the value of the dispense sensor 503. When a reward is detected, the input status may be HIGH. When the Arduino sees this, it exits the dispense loop and turns on the dispense light 504. This signals to the subject under test that it can retrieve a reward. The reward controller 103a also sends the main controller 102 a “reward detected” message. If a reward is not detected and the timeout is reached, the system returns “reached timeout” via a serial bus.

To make the system more reliable, the reward controller 103a may be configured to check if the dispense pathway 620 is jammed. Before dispensing a reward, the reward controller 103a will obtain a reading from the dispense sensor 503. In some aspects, a HIGH read indicates that the pathway 620 is jammed, and the controller may send a “dispenser jammed” indication to the main controller 102. The system may also reads from a dispenser door sensor 503a to see whether the subject has picked up the reward. This means that the main controller 102 can query the dispenser door sensor 503a data both continuously and upon command. If the dispenser door 501 is touched, in some aspects, the status of dispenser door sensor 503a will change to HIGH and the dispense light 504 may be turned off by the main controller 102.

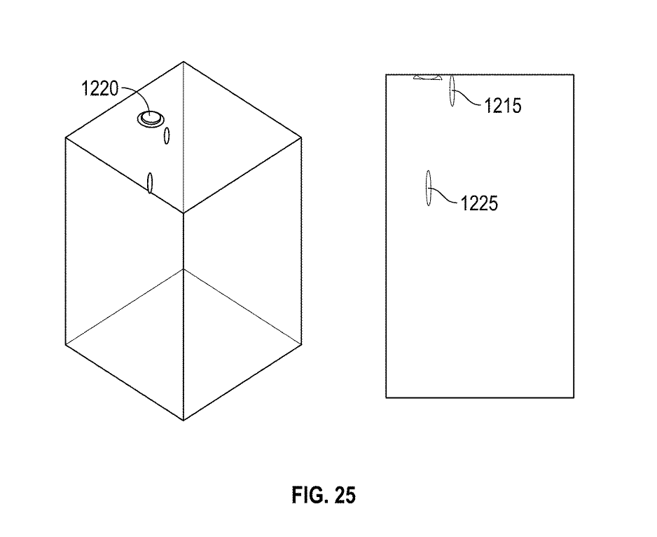

FIG. 25 illustrates an exemplary positioning for a noise controller speaker 1220, microphone 1215, and sound meter 1225. As shown, the location of the microphone 1215 close to the speaker 1220 allows the microphone 1215 to have maximum sensitivity to the volume of the white noise. In certain embodiments, the sound meter 1225 is positioned such that its data will closely approximate the experience of the test subject. As a result, the sound decibel value given by the meter 1225 is an estimate of the sound level heard by the test subject. In some aspects, the meter 1225 may also be positioned such that it is close enough to the environment camera 75 so as to track the video stream.

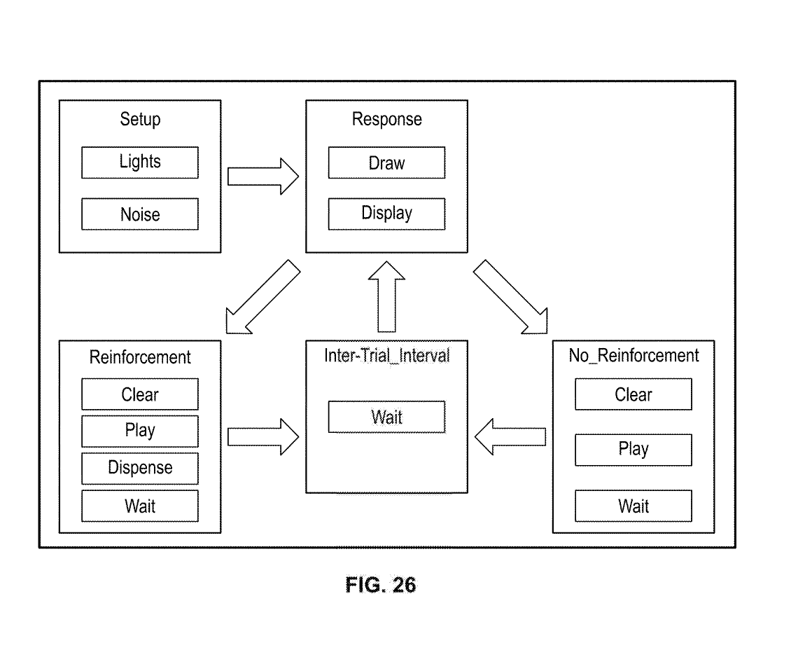

FIG. 26 shows a flowchart of an example concurrent discrimination test protocol. The boxes in the flowchart are called intervals. These determine the state of the experiment. Inside each interval is a group of actions, which describe what the hardware should be doing during a given interval. Transitions, which are depicted as the arrows between intervals, are the way the experiment moves from interval to interval. Which transition the experiment takes depends on which criteria have been met at that point in the experiment (e.g., which image has been touched, whether or not all actions have been completed, etc.).

For example, a concurrent discrimination experiment may begin with a setup interval, in which the environment is adjusted to a predetermined state. The adjustment may include adjusting light levels, adjusting noise (e.g., via a white noise generator), adjusting temperature, scent, or the like. A sensor may be provided to detect a current level of an environment factor (e.g., light, noise, temperature, air quality). The adjustment may be performed using the sensed level for the factor.

After the setup, the concurrent discrimination experiment may begin with an interval containing two actions. The first action may select two images, and the second may display both images on a screen. If the subject chooses the first image (the “correct” one), the experiment may then move via a transition to an interval containing actions that play a reward tone and give a reward. If the subject chooses the second image (the “incorrect” one), the experiment may move to an interval containing an action that plays a non-reward tone. Regardless of which of the two intervals the experiment may be in, the experiment, in some implementations, waits for all actions in the interval to finish and then transitions to a new interval. In this interval, the experiment may wait for a certain number of seconds before transitioning back to the first interval and starting the cycle over again.

The intervals and actions performed during an experiment involve complex coordination of various testing hardware such as the screen, an input device, the audio output, a reward dispenser (e.g., pellet dispenser, candy dispenser, fragrance dispenser, printer, gel dispenser, coin dispenser, etc.), subject monitoring hardware (e.g., camera, microphone, electrodes) or the like. Properly specifying the protocol such that it can be executed in the same way across testing hardware is desirable to ensure reliability in the test results.

Furthermore, the testing hardware may deteriorate over time. For example, the chamber lights may be made with LEDs that get dimmer over time. As such, over time, the light generated during a first experiment may be different than the light produced during a second experiment. To ensure adherence to the specified protocol, it is desirable to adapt execution of test protocol to account for local variations in the hardware executing the test. While the example discussed references chamber lights, it will be appreciated that characteristics of other testing hardware such as audio output device, video display, touchscreen, or other input or output testing devices, may be detected and used to adapt execution of a text protocol.

Finally, as discussed above, in current testing systems, the protocol is specifically tied to the hardware executing the test. Any changes to the hardware may necessitate a significant change to the protocol definition.

To facilitate reliable test execution and control, a configuration language may be used. The configuration language may allow researchers to describe an experiment's flowchart and run the experiment, independent of the underlying hardware used to perform the test. The structure of the language itself may mimic a flowchart, where each block of text corresponds roughly to an interval in the flowchart. For purposes of explanation, the application will continue to use the example of concurrent discrimination to explain the syntax, the words used in the language. It will be appreciated that other forms of a configuration language may be implemented to provide reliable test execution and control.

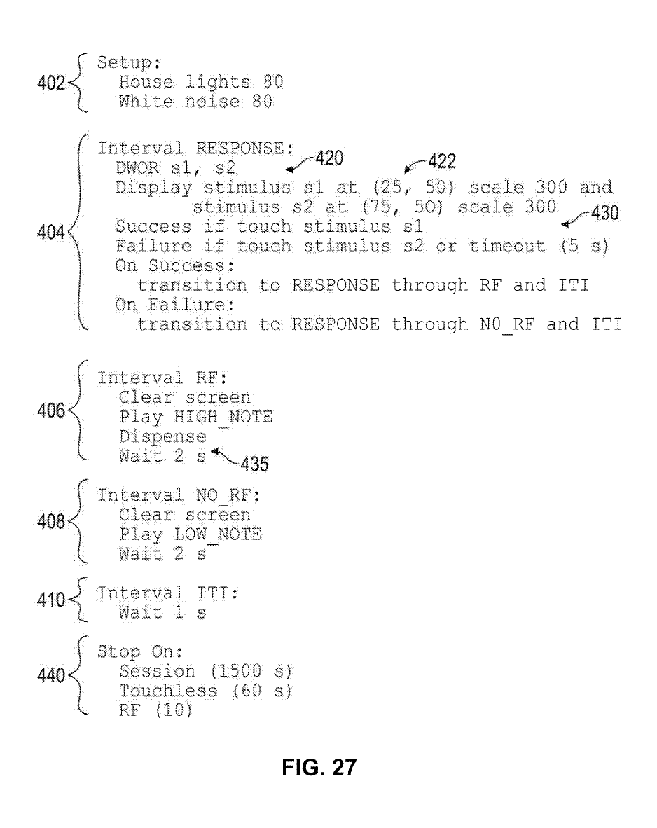

FIG. 27 is a listing illustrating an example protocol configuration for a concurrent discrimination experiment protocol. The example listing of FIG. 27 begins with a setup interval 402. The setup interval 402 may be run when the experiment first starts. The setup interval 402 provides configurations for initializing testing hardware to settings that will remain constant for the duration of the experiment, such as lights and white noise.

The listing also includes four intervals 404, 406, 408, and 410. Each interval may be assigned a name both for readability and later reference in the protocol configuration. For example interval 404 is given the name “RESPONSE.” After an interval is defined, the specific experimental protocol activities to perform for the interval may be specified. These activities may be referred to as actions. Actions are the discrete operations performed each time the experiment reaches the interval containing them. Action configurations may be expressed as a keyword or keyword pair, followed, in some instance, by one or more parameters of the action. The interval 404 includes two actions: action 420 and action 422.

An interval may also include a transition configuration. Within a protocol listing, the transition configuration may be specified at the end of an interval. The interval 404 includes an input driven transition 430. The input driven transition 430 is based on input from the testing hardware. The configuration includes a definition of groups of named conditions (e.g., “success” and “failure). The configuration then references each of the named condition to specify how to transition when the associated condition is met. Each transition may include an ending interval (which should be another input interval), and optionally one or more transition intervals to go through first. An interpreter may be included in the central hub, such as the central hub 105 shown in FIG. 1B, to coordinate application of the configuration across the child controllers, such as the child controllers 103a-g shown in FIG. 1B, associated therewith and, in some instance, communicate information to another entity such as to a meta hub. The interpreter may be implemented as an interrupt-driven state machine. Exemplary features of such state machines are provided in Schneider, “Implementing Fault-Tolerant Services Using the State Machine Approach: A Tutorial,” ACM Computing Surveys, vol. 22 no. 4 (December 1990) the entirety of which is hereby incorporated by reference.

A time-based interval transition 435 is specified for the interval 406. The interval 406 performs the specified actions (clear screen, play note, and dispense) and then holds for a period of time specified in the time-based interval transition 435 before continuing execution of the experimental protocol. A timer may be initiated by a central hub upon processing of the time-based interval transition 435. When the time indicates the period of time has elapsed, the central hub may continue processing the experimental protocol.

The listing shown in FIG. 27 also includes exit criteria 440. The exit criteria 440 specify the conditions during which the experiment should end. The test runner may be configured to evaluate the exit criteria 440 and compare a state of the experiment, testing equipment, subject, or combination thereof to determine whether the exit criteria 440 are met.

Various functions may be implemented to allow dynamic configuration of the exit criteria 440. One exit criterion may be a session timeout. Session timeout specifies a maximum length of time to perform the entire experiment. When the test begins, a session timer may be initiated. The experiment terminates after the indicated time limit has elapsed. Another exit criterion may be a touch timeout. A touch timeout may be used to protect against a lack of interaction from the subject. A touch timer may be initiated as part of a display action. If the subject does not touch the screen for the specified timeout length, then the experiment may terminate. Another exit criterion may be an interval count exit criterion (e.g., RF (10)). Once a specified interval has been entered a number of times equal to the interval count exit criterion, the experiment may terminate. The interval count may identify the number of intervals to perform, the number of correct intervals to perform, or the number of incorrect intervals to perform. It will be appreciated that, as shown in the listing of FIG. 27, multiple exit criteria may be specified for controlling a test.

To permit flexibility in hardware components that may carry out specified actions, actions may be implemented as an abstraction which is resolved at runtime by the central hub. Rather than the intervals needing to know how to perform each possible action, the actions themselves may store this information. In one implementation, an action identified in the experimental protocol configuration may be associated, at runtime by the central hub, with a hardware command that is transmitted to specific testing hardware each time the action is supposed to be performed during the experimental protocol. Examples of actions include those for commands (e.g., execution of a hardware function) and those for choosing stimuli for the experimental protocol.

Command actions may specify relevant information about something the hardware should do. The central hub is configured to reserve memory sufficient to maintain the specified information for the interval. For example, a command action “DISPLAY” may be used to instruct a testing display hardware to display the information specified in the configuration. The display command action may include configuration specifying one or more of: what to display, where on the display to present the information to be displayed, and adjustments to the content to display. In some implementations, these values may be static (e.g., a specifically defined image, sound, etc.) or dynamic. In the dynamic implementations, the central hub may reference a value obtained or generated by another action such as an action for choosing a stimuli. When the command action is processed by the central hub, the configuration information is may be transmitted to the hardware to handle. The central hub may perform conversion of the configuration information to conform to a format understandable by the target hardware. For example, one testing station may include a USB monitor while a second testing station may include a High-Definition Multimedia Interface (HDMI) display device. In such implementations, to achieve the same the presentation, the display configuration information may be tailored such that a USB version is transmitted to the USB monitor and an HDMI version is transmitted to the HDMI display device.

The command action may be further tailored to account for localized differences between the testing hardware. For example, the brightness of the HDMI monitor may be higher than the brightness of the USB monitor. In such instances, the display action may be modified to adjust a brightness configuration for the commands to ensure the presentation via either monitor is at the level specified in the protocol. For example, a camera may capture an image of a test pattern on the display and transmit this to the central hub. The central hub may then compare a characteristic of the test patterns (e.g., brightness) with the expected value. If there is any difference, a correction factor may be stored for the display. The central hub may be configured to then apply this correction factor for a display command action targeting the display.

Testing hardware may be controlled through a communication. A communication, as used in this application, generally refers to a single message generated by the central hub and transmitted to one or more hardware devices. Some hardware devices may be configured to provide responses or detect input values. In such implementations, the hardware devices may provide a communication to the central hub.

Each communication may include a type and a sequence number. Each type of message may be associated with additional type-specific data of its own. By looking at the type of a communication, the recipient of the communication knows what additional fields to look for and how to interpret the configuration values included therein. The sequence number included in a communication is used to uniquely identify communications, since two communications could potentially be otherwise identical. Sequence numbers may further identify which side sent a given message. For example, the central hub may include odd sequence numbers in communications originating at the central hub, while communications sent by testing hardware may each include an even sequence number.

The system may support multiple communication types, each with a unique purpose. One communication type may be a command. Commands indicate an instruction to adjust the state of one or more testing hardware devices. Commands are generated by the central hub for the targeted hardware. The command includes information to instruct the hardware to perform tasks, like displaying images, playing sounds, or dispensing pellets. The central hub converts the configuration file into specific commands. Commands may have an action, one or more parameters, or a combination thereof. The action may describe an adjustment to the state of the testing hardware. A parameter may specify action-specific data representing different inputs to the action, such as what image to display or what frequency tone to play.

Another communication type may be an acknowledgment. Acknowledgements may be used to confirm that the testing hardware is behaving as expected. Rather than simply confirming receipt of the communication, an acknowledgement indicates that, in addition to a command being received, it executed correctly. In some implementations, for every command that is sent, an acknowledgement may be sent back. The acknowledgement may include the sequence number of the command it is acknowledging. In such implementations, the central hub can determine which command each acknowledgement corresponds to. The acknowledgement may also contain a code indicating what error, if any, occurred. For example, if one attempted to dispense a pellet, the hardware may respond with an acknowledgement with error code 703, which indicates that the pellet dispenser jammed.

For the experiments to be dynamic the central hub may be configured to receive an indication of when the test subject is interacting with a hardware device. An input is another type of communication. Input communications may be sent by testing hardware whenever the hardware is interacted with. Like command communications, input communications may include a value indicating an action informing what occurred, one or more parameter values describing the occurrence, or a combination thereof.

Communications may include one or more encoded messages. To ensure efficiency and flexibility, the encoding may be a lightweight encoding such as JavaScript Object Notation (JSON). The communication encoding is preferably machine readable and easily transmitted. For example, in implementations where communication reliability is favored, the Transmission Control Protocol (TCP) may be used. While TCP and similar protocols cannot overcome significant failures within a network, in general even communication over a good internet connection can be lossy, it uses a variety of methods to get around this normal loss. TCP establishes a connection between a server and client and ensures that while that connection remains open, data can be transmitted across it as though it were a reliable byte stream between the two machines. Thus, TCP not only ensures our messages get delivered, but also that they are received in the same order they are sent in.

Stream-like connections, such as those according to TCP, can pose a problem for systems with discrete message communications. When multiple messages arrive within a short time span of each other the receiving device may be unable to determine the boundary between the messages. One way to detect boundaries is to include a separator character or string. In real time systems, such as testing systems, this can be inefficient because detection of the character or string requires scanning each new byte as it comes in to identify the separator. This technique also requires some way of assuring the separator will not appear within the data itself. Another solution is to send a header of a set length (e.g., a standard 4 byte integer) which states the size (such as in bytes) of the following message. On the other end, to receive a message first just the header is received and looked at. Then the remaining message (of now known size) is received and parsed, with the knowledge it will be a complete message.

The central hub and the test hardware may include a communication manager configured to maintain a communication channel for receiving and sending messages. Maintaining the communication channel may include one or more of connecting, disconnecting, receiving, and sending. The central hub may be executing multiple tests concurrently. Similarly, a main controller 102 for testing equipment may be controlling multiple testing hardware units concurrently. As such, the send function for the communication manager can be invoked by any entity under control of the central hub or controller. To coordinate communications, the manager may regulate messaging by a lock to ensure only one entity is sending at once. Without this lock, multiple messages might attempt to send simultaneously, and their content could become intermixed, rendering the messages unintelligible.

The communication manager may be in communication with a response manager configured to track responses to ensure all commands receive acknowledgements, generate and/or send an error (to trigger event-based error handling) if one does not return in a timely manner. A timely response may be preconfigured or dynamically determined using the testing protocol, test hardware configuration, test subject, or the like. In some implementations, the response manager may be configured to forward responses to the appropriate event-based responders. Event-based responders may include testing hardware, data store, or a combination thereof.

The system may support multiple communication types. Three examples of types of communication the hardware may send include: inputs, acknowledgements, and notifications. Inputs may be parsed for relevance, and acted on if appropriate. Acknowledgements, once confirmed, can be ignored unless they specify an error. Similarly, notifications indicate something that needs to be acted upon. Because the system response to each communication type may differ depending on the testing hardware, event-based managers may be provided to interpret the communication and generate the proper testing hardware state adjustment.

An error monitor may listen to incoming communications and identify acknowledgement and notification events. For each, if a non-success error code is included in the communication, a graceful exit command may be generated. This may include shutting down, resetting, or otherwise reverting the state of one or more hardware devices in response to the ending of the experiment.

It may be desirable to include a notification monitor. The notification monitor may be configured to listen to incoming communications and identify notifications. In some implementations, an interface may be provided for presenting the relevant message. In some implementations, the message may be used to generate an alert such as a send text message to the scientists running the test. For example if an error condition is reported, such as light level being too low, an alert message may be transmitted to a predetermined location. Another example of a notification that would generate a text message alert would be if the test ends prematurely due to subject inactivity. In some implementations, the message may cause the initiation of an application on a communication device receiving the message. The application may then be configured to obtain and/or present the results of the test, current status for the testing hardware, or other real-time or collected information related to the test or testing hardware.

It may be desirable to include an event monitor. The event monitor may be configured to check all input communications, and package them in a specialized event data structure. The data structure may be implemented to facilitate comparison of instances to one another not just for equality and similarity but also for subset equality. The event monitor may be configured to forward each event data structure to the currently active interval processor. If the interval is an input interval, the processor may be configured to compare each event data structure received to a list of events they are supposed to transition on, and transition if appropriate.

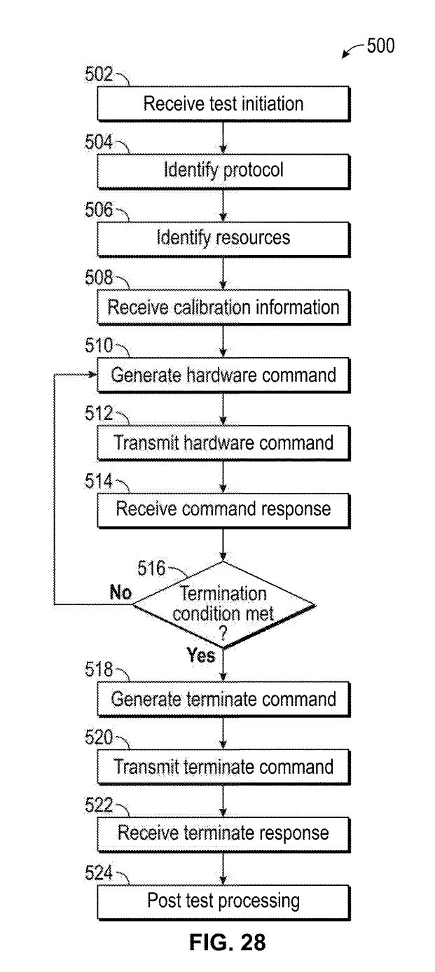

Cognitive testing typically requires storing information about how an experiment runs for later analysis. A logger may be included to keep track of errors, inputs, and interval changes, along with information on the timestamp for each, and the test subject participating in the experiment. The logger may store the information in a file or data store. In some implementations, it may be desirable to record which testing unit was used to perform the test. FIG. 28 illustrates a process flow diagram of an example method of cognitive testing. The method 500 may be implemented in whole or in part by one or more of the hardware devices described in this application.

At block 502, the method 500 begins with the receipt of a test initiation. The test initiation may be a message transmitted to a central hub. The test initiation may include information identifying the testing protocol to initiate. The test initiation may include information identifying a specific testing unit to initiate the test on. The test initiation may include information identifying a subject for the test. The central hub may be implemented in the central hub 105 and/or within a meta hub. In some implementations, such as where a single test unit is deployed, the central hub may be implemented in the main controller 102.

Receipt of the test initiation may be achieved via a data communication channel with the central hub. The channel may be a wired or wireless communication channel. In some implementations, the initiation may include the specific values for the information included in the initiation message. In some implementations, the initiation may include a pointer to the values such as a unique identifier which can be used by the central hub to query a data store for the information.

Upon receipt of the initiation, at block 504, the test protocol configuration is identified. The experimental test protocol may be stored in a data store or within a file system. Using the information included in the initiation received at block 502, the central hub may obtain the protocol to be initiated. For example, the initiation may include the name and location of a file containing the protocol configuration. The file may include a protocol configuration similar to that shown in FIG. 27.

Having identified the protocol, the method 500 may also include, at block 506, identifying resources for performing the protocol. The resources may include the testing hardware. For example, a protocol may call for a specific element of testing hardware such as video or reward dispenser. In such implementations, the testing units with the desired hardware may be identified. The specific testing unit may be specified in the initiation and/or protocol. However, in some implementations, it may be desirable to allow the central hub to identify the test unit best suited to perform the protocol based on other scheduled tests, available hardware, location of the hardware, location of the subject, and the like.

Other resources which may be identified for a protocol may include stimuli. In some protocols, media may be specified as the stimulus for the subject. The central hub may be configured to ensure the testing hardware that will perform the test has the specified media (e.g., image, video, sound file) available. If the central hub determines the resources are not accessible by the testing hardware, the central hub may initiate a process to transfer the specified media to a location that can be accessed by the testing hardware.

At block 508, the central hub may receive calibration information from the testing unit. The calibration information may include sensor data from the testing hardware indicating a state of one or more testing hardware device include in the testing unit. The calibration information may include one or more of sound, light, weight, scent, humidity, or temperature information for the testing unit or a specific element of testing hardware. The calibration information may be generated by taking absolute measurements (e.g., temperature). The calibration information may be generated by presenting a known output and measuring the actual output from an element of testing hardware. For example, a camera may be included in the test unit to capture an image of a touchscreen display also included in the test unit. A test pattern may be displayed on the touchscreen display and, while displayed, the camera may capture an image of the touchscreen display. This image may be then be compared to the test pattern to determine variance from the desired output for the touchscreen display. Such variance may include brightness, image alignment, damage to the screen itself, as evidence by a visual disruption in the captured image, or the like.

The calibration information may be stored by the central hub and used during execution of the test protocol to adjust configurations to achieve the specified protocol. At block 510, the central hub uses the test protocol configuration to identify a hardware command to issue. The hardware command identification may include identifying one or more testing hardware elements to adjust and a format for the command to cause the desired adjustment. For example, the configuration may indicate turning a light to cast 50 lumens of blue light. The central hub may retrieve from a data store the light adjustment commands for the light included in the test unit. The light adjustment commands may accept parameters to perform the adjustment. The parameter may identify a control amount for the hardware. The central hub may be configured to convert specified values in the protocol configuration to a unit accepted by the target testing hardware. For example, the light may accept light quantities specified in watts rather than lumens. In the example configuration specifying 50 lumens of light, the central hub may convert the lumens to watts.

The central hub may also be configured to apply a correction to a hardware parameter value using the correction information. For example, if the calibration information for the light received from a light sensor included in the test unit, indicates that a test requesting 50 watts produced an effective output of 48.2 watts, a correction factor of 1.8 watts may be applied when specifying the quantity of light to display for that specific light. While the correction factor described herein is the difference between the requested and produced output, some output discrepancies may be non-linear and be generated using more complex relationships (e.g., exponential relationships, logarithmic relationships, degradation models, predictive models).

Having generated the calibrated hardware command, at block 512, the hardware command is transmitted to adjust the testing hardware. The command may be transmitted from the central hub to a main controller 102 which may, in turn, provide the command to a testing hardware element.

While generating the hardware command may be performed at the central hub at the central hub 105 or meta hub, in some implementations, it may be desirable for the central hub at the central hub 105 or meta hub to transmit the protocol configuration to the main controller 102. In such implementations, the main controller 102 of the testing unit may then generate specific hardware commands to adjust the state of the specified testing hardware to follow the specified protocol.

At block 514, a command response may be received. The command response indicates that a given configuration command was received and, in some instance, whether the adjustment was successful, as discussed above.

At block 516, a determination is made as to whether a protocol termination condition has been met. The determination may be made using values included in a command response in comparison with termination conditions specified in the protocol configuration. The determination may be made using timing information, such as elapsed time from the initiation of the test. The determination may be made using a combination of the response and timing information.

If the determination at block 516 is negative, the method 500 returns to block 510 to generate the next hardware command to adjust the testing unit according to the protocol. If the determination at block 516 is affirmative, the method 500 continues to block 518 to generate a terminate command. The terminate command may be generated for the test unit and/or a specific testing hardware element included therein. The terminate command ensures that the test unit is gracefully and safely brought into a resting state at the end of the test. The terminate command may include changing power state, changing an output (e.g., image, sound) for a hardware element, adjusting an environment characteristic (e.g., temperature, light, scent, humidity), or a combination thereof. In generating the termination command, the calibration information may be applied as described with reference to block 510.

Having generated the termination command/commands, block 520 and 522 transmit the command and receive a response (respectively), in a similar way as described for block 512 and block 514 (respectively).

In some implementations of the method 500, at block 524, post-test processing may be included. Post-test processing may include storing command responses which, in some implementations, include the test data. Post-test processing may include parsing the responses to store in a data store, information about the testing unit, the specific test execution, calibration information, or other data generated or received during the test. Additionally, detailed information may be stored for each interval transition; for system setup information; for script end conditions; and for each command with parameters and child controller response settings and timing. This information can be used to analyze the result of the test. This information can be used to identify testing hardware that may need repair. For example, the central hub 105 may include a maintenance monitor configured to listen for calibration information from various testing hardware. If the calibration information reaches a predetermined threshold, the maintenance monitor may generate an alert to indicate the testing hardware may be malfunctioning. The threshold may be a single value comparison such that any value which deviates from the threshold would trigger the alert. In some implementations, the comparison may be an average, a moving average, or other aggregation of calibration data.

The alert may be transmitted to a maintenance scheduling system and include information identifying the testing hardware, testing unit, time of detection, and/or a combination of this information. In some implementations, the alert may be transmitted to central hubs and prevent future initiations of protocols that may utilize the identified testing hardware until the testing hardware is checked.

The following are the commands that can be sent to the hardware to adjust its state. Commands are typically implemented as communications. As such, the commands include a type field and a sequence number.

The method 500 may generate various commands. The commands may be provided to allow a researcher to specify protocols without worrying about the underlying hardware implementing the actions. As such, a variety of commands may be implemented to afford the proper control for the desired protocols.

A show command may be included to configure a display to present a set of images. The show command may include a parameter to specify images to show. The parameter may specify a list of dictionaries describing the images, not just a single dictionary. The show command may include a position parameter to specify where a given image should be shown. The unit is a parameter that may be included to indicate how to measure the position. The unit may be specified in “%” means percent of screen, from 0 to 100. The unit may be specified as coordinates. Coordinates may be specified as, where is the top left, increasing x values progress to the right, and increasing y values progress downwards. The show command may also include a display size parameter to specify the size of the image to be displayed. As with the position parameter, the size parameter may include a unit. The size unit may be specified as a percentage of original image dimensions. Height and width may be specified according to the unit indicated in the size unit (e.g., cm for centimeters, px for pixels). Each new show command may cause the display to clear whatever was previously showing.

A dispense command may be used to control a reward dispenser such as pellet dispenser. The dispense command may be provided without parameters. In such instances, one reward is dispensed. In some implementations, it may be desirable to dispense a quantity. In such implementations, the dispense command may include a quantity parameter indicating how many times to trigger the dispensing hardware. In some protocols, a dispenser may be used to dole out a variety of rewards. In such protocols, the dispense command may include an indicator of which reward to dispense. A tone command may be included. The tone command causes an audio output to play a specific tone. The tone command may include parameters to specify one or more of: the frequency (e.g., hertz), volume (e.g., decibels, percent of max), and/or duration for playing the tone. As discussed above, the tone command included in the protocol configuration may be adjusted based on the calibration information for the target tone playing hardware.

A noise command may be included. The noise command causes a white noise generator to present specific background noise. The noise command may include parameters to specify one or more of: the volume (e.g., decibels, percent of max) or a noise audio file.

A set light command may be included to adjust lighting within the testing unit. The set light command may include parameters to specify one or more of: light state (e.g., on or off), light intensity, light brightness, light color, or other variable aspect of the lighting.

A command to trigger an indicator light may be included. A testing hardware element included in a test unit may include an indicator light. The indicator light may be used to visually confirm that the hardware is functioning. For example, to ensure the test unit is properly communicating, a series of commands to trigger the indicator light on each hardware element may be transmitted. A camera may be used to sense whether the light was triggered or not and, based on the sensed result, a test may be initiated (if successful) or aborted (if one or more hardware elements did not function as expected).

A command to initiate a video stream for the testing unit may be included. The command may start streaming from a video camera in the testing unit. The command may include parameters to specify a port on which to stream, frame rate, stream resolution, or other video capture parameters. A stop streaming command may be included to terminate the stream for a testing unit.

Some protocols may also include receiving input from the subject. Inputs reflect an interaction by the subject with a hardware element included in the test unit. For example, a touchscreen device may be used to receive inputs. An indication of a touch input may be transmitted whenever the screen is touched. If the screen was presenting an image, the indication may include an identifier for the image that was touched. If the touch was on a blank part of the screen, the indicator may be empty or include a known “null” value. The indication may include a location parameter specifying where the screen was touched. The location may be specified in a predetermined unit (e.g., centimeters, inches, pixels) relative to a predetermined position on the screen (e.g., the top left, bottom right, screen center). In some implementations, it may be desirable to reference images shown in the display using an index value. In such implementations, an index parameter may be included to identify the index of the image that was touched in response to the related show command image parameter.

Another example of an input device is a lever. A lever press action may be generated whenever the lever is pressed. If multiple levers are included, a parameter indicating which lever was pressed may be included in the action as a parameter.

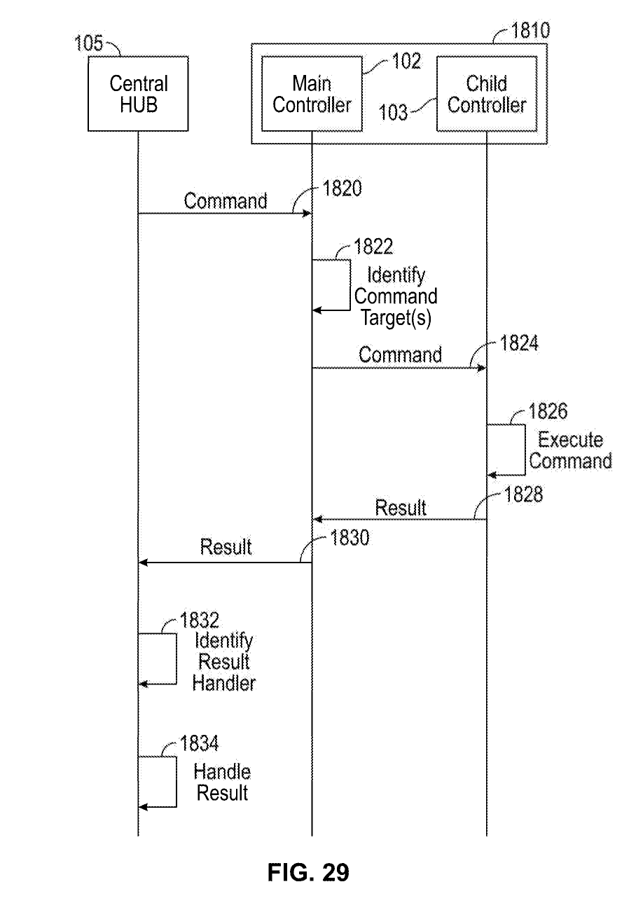

FIG. 29 shows a message flow diagram of protocol command execution. The message flow shown in FIG. 29 includes messages exchanged between exemplary entities selected to highlight certain features related to protocol command execution. It will be understood that fewer or additional entities may be included to achieve a similar result.

FIG. 29 shows a central hub 105 which is in data communication with a testing unit 1810. The testing unit 1810 may include a main controller 102 and a child controller 103. The child controller 103 may be configured to adjust a function of a device within the testing unit 1810 such as a display, a pellet dispenser, a speaker, a camera, or other similar devices described in this application.

Via message 1820, the central hub 105 may provide a command to the main controller 102. As discussed above, the command may indicate an instruction to adjust the state of one or more testing hardware devices. The command may be generated by an interpreter executing on the central hub 105. The command may include information to instruct the hardware to perform tasks, like displaying images, playing sounds, or dispensing pellets. However, because the testing unit 1810 may have a specific configuration or unique hardware, it may be desirable to allow the central hub 105 to transmit commands to the main controller 102 which is configured to translate the command into specific hardware instructions for the local provider of the desired action.

As such, via message 1822, the main controller 102 identifies one or more command targets. In some implementations, the command may indicate two pieces of hardware be adjusted. For example, if the command is to play a sound, it may be desirable to play the sound via two audio output devices. In such implementations, two targets would be identified for the specified command. In identifying the target, the main controller 102 may forward or generate a new command based on the command included in the message 1820. This command is then provided to the child controller 103 via message 1824. The child controller 103 receives the message 1824 and uses the command to adjust its configuration and execute the command via message 1826.

As shown in FIG. 29, the child controller 103 generates a response message 1828 indicating a result of the command. The result may indicate a successful dispensing of a pellet. The result may be a measured value such as from an environmental sensor.

The main controller 102 may transmit a message 1830 to the central hub 105 indicating the result. The main controller 102 may forward the response message 1828 or generate a new message 1830 based on the response message 1828. For example, the main controller 102 may be configured to translate the response message 1828 into a standardized format which is independent of the physical device that generated the result.

The central hub 105 via message 1832 may identify a result handler for the result identified by the message 1830. The message 1832 may include querying a data source for the proper handler or chain of handlers for the result. For example, if the result is a touchscreen response, a handler may be identified to translate the coordinates of the touchscreen response into a logical result for a subject and generate a log entry indicative of the response, or initiate the transition to a different interval depending in where the touch occurred. Via message 1834, the identified handler is provided the result and the result is handled thereby. It will be appreciated that in some implementations, the handler may be a remote handler. In such instances, the central hub 105 may forward the result to another entity, such as a meta hub 132, for handling.

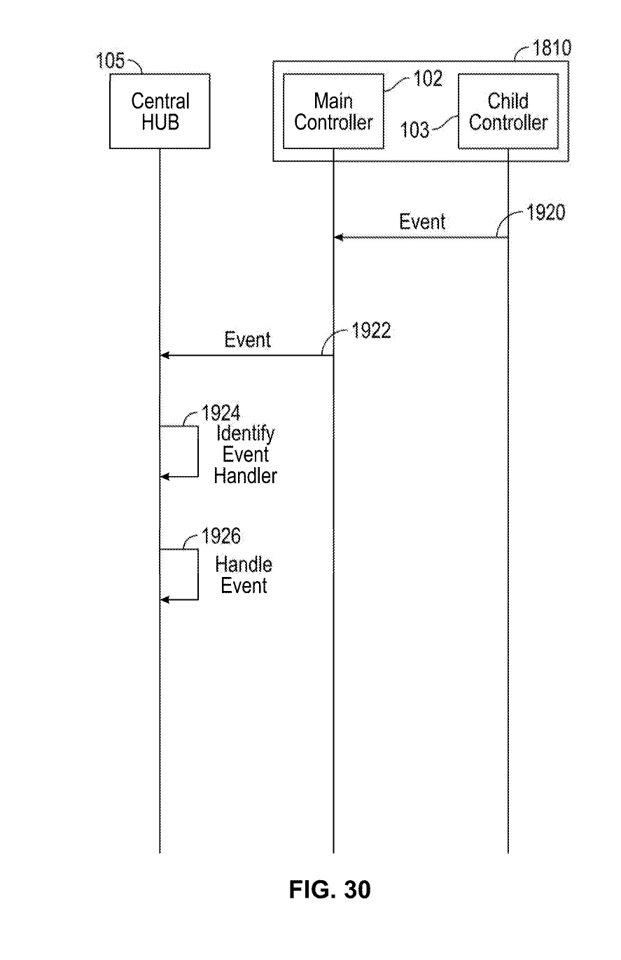

FIG. 30 shows a message flow diagram of child controller event handling. The message flow shown in FIG. 30 includes messages exchanged between exemplary entities selected to highlight certain features related to child controller event handling. It will be understood that fewer or additional entities may be included to achieve a similar result.

FIG. 30 shows similar entities as shown in FIG. 29. Whereas in FIG. 29, the central hub 105 caused the child controller 103 to generate a result and that result was then transmitted back to the central hub 105 for handling, not all messages generated by the child controller 103 may be in response to a command from the central hub 105. FIG. 30 demonstrates how the child controller 103 may generate event messages without prompting and how these messages may be handled.

A message 1920 may be generated and transmitted by the child controller 103 to the main controller 102. The message 1920 may indicate, for example, a touchscreen event or a temperature reading. The message 1920 may be generated while the testing unit 1810 is performing a test. In some implementations, the message 1920 may be generated at a time when the testing unit 1810 is not performing a test.

In some implementations, the message 1920 may cause the main controller 102 to take a corrective or responsive action. For example, if the temperature reading is above a predetermined threshold, the main controller 102 may activate a cooling unit or power down the entire testing unit 1810.

In addition, or in the alternative, the main controller 102 may transmit a message 1922 indicating the event to the central hub 105. The message 1922 may be a forwarded version of the message 1920. In some implementations, the main controller 102 may generate the message 1922 based on the message 1920.

The central hub 105 may then, via message 1924, identify an event handler for the event. The message 1924 may include querying a data source for the proper handler or chain of handlers for the event. For example, if the event is a touchscreen response, a handler may be identified to translate the coordinates of the touchscreen response into a logical result for a subject and generate a log entry indicative of the response. Via message 1926, the identified handler is provided the event and the event is handled thereby. It will be appreciated that in some implementations, the handler may be a remote handler. In such instances, the central hub 105 may forward the event to another entity, such as a meta hub 132, for handling. Handling the event may include transmitting one or more commands to a child controller 103 such as shown in FIG. 29.

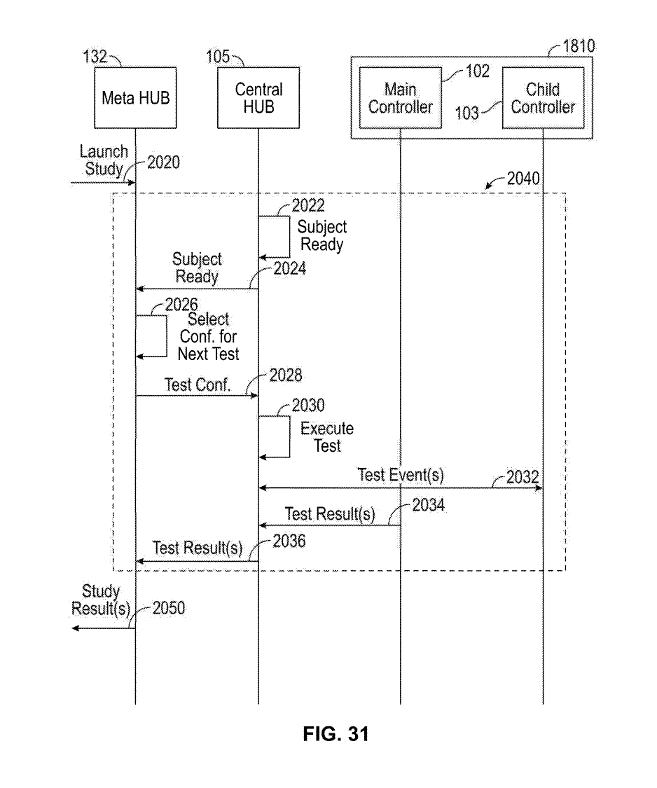

FIG. 31 shows a message flow diagram of protocol command execution for a study. The message flow shown in FIG. 31 includes messages exchanged between exemplary entities selected to highlight certain features related to protocol command execution for a study. It will be understood that fewer or additional entities may be included to achieve a similar result.

FIG. 31 shows similar entities as shown in FIG. 29. Included in the message flow diagram of FIG. 31 is a meta hub 132. The meta hub 132 may be configured to control execution of a study. A study may generally refer to a series of tests conducted with one or more subjects. The series of tests may be linear (e.g., executed in series after completion). In some implementations, the series may be non-linear. For example, the series may include conditional logic whereby results from the performance of a first test influence which test is administered next.

Via message 2020, a study may be launched. The study may be predefined or defined by the message 2020. As discussed, a study may include a series of tests and a desired number of subjects to which the tests will be administered. The study may be defined using a protocol configuration such as a script. Tests within the study may be administered according to a protocol configuration such as those described above.

The central hub 105 may receive an indication message 2022 identifying a subject enrolled in the study is ready for a test. The indication message 2022 may be received from an electronic device within the testing unit 1810 such as a radio frequency identification reader. In some implementations, the indication message 2022 may be received from an application (e.g., web-application) once the subject is in place for the next test.

Via message 2024, the central hub 105 indicates to the meta hub 132 that the subject is ready. The message 2024 may include an identifier for the subject. Using this identifier or other information provided via the message 2024, the meta hub 132 select the protocol configuration for the test to be administered to the identified subject within the study. The selection may be performed via a look-up in a data store. The selection may be based on a study protocol configuration which includes logic as to which test protocol configuration should be provided for the subject within the study.

The identified test protocol configuration is provided to the central hub 105 via message 2028. The central hub 105 may then execute the test protocol configuration as described herein, such as with reference to FIG. 28 FIG. 28. During the test, commands, command results, and events are generated and communicated between the central hub 105 and the testing unit 1810. The communications may be performed using one or more test event messages 2032. Eventually, the test will come to an end and test results may be provided to the central hub 105 via message 2034. The message 2034 may include an identifier for the subject, an identifier for the test protocol configuration, an identifier for the study protocol configuration, date information, time information, environmental sensor data (e.g., temperature, noise, light level), image data, audio data, video data, command log and command response timing, interval transition times, or other information collected by the testing unit 1810 during the test. The test results may be provided during the test rather than after completion. In such implementations, the incremental results may be stored, such as at the central hub 105, until the test is completed. Upon completion, the results may be compiled into a final results set for transmission to the meta hub 132.

Results for the test may be transmitted to the meta hub 132 via message 2036. The message 2036 may include all the results within the message. In some implementations, the message 2036 may provide a pointer to the results, such as a uniform resource locator of a network location where the results may be obtained. Messages 2022 through 2036 may be used to execute a test for a subject within the study. Collectively these messages may be referred to as subject test messaging 2040. The subject test messaging 2040 may be repeated to allow testing of the same or additional subjects according to the study's protocol.

The study protocol may indicate a termination condition such as a number of test to perform per subject, or a desired response rate for particular test activities. Upon detection of a termination condition, the meta hub 132 may generate one or more study results 2050. The study results 2050 may be generated by combining or otherwise processing the individual test results received from the central hub 105.

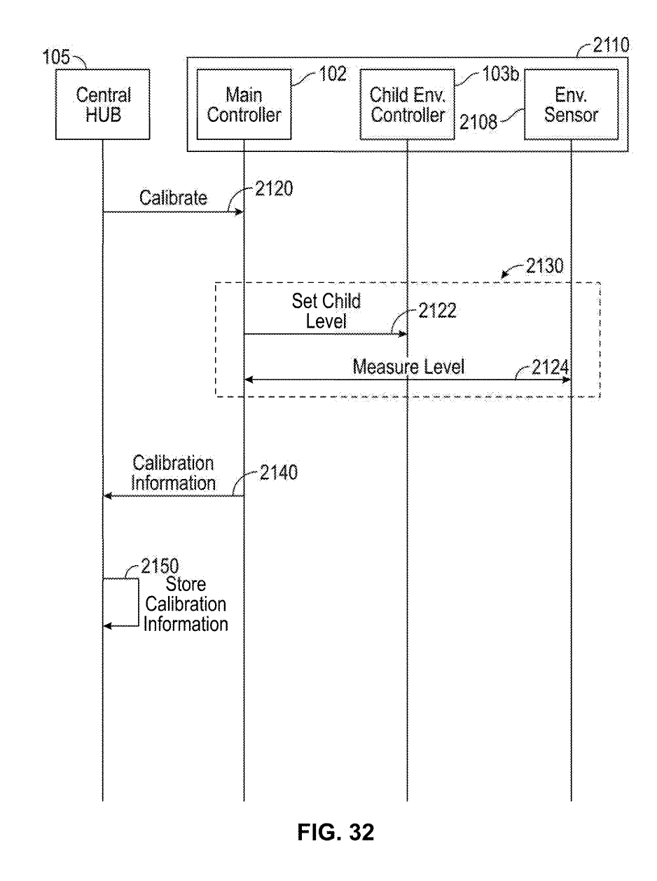

FIG. 32 shows a message flow diagram of dynamic environmental calibration. The message flow shown in FIG. 32 includes messages exchanged between exemplary entities selected to highlight certain features related to dynamic environmental calibration. FIG. 32 includes a central hub 105 in data communication with a testing unit 2110. The testing unit 2110 includes a main controller 102, a child environmental controller 103b, and an environmental sensor 2108. The child environmental controller 103b may be an electronic device configured to adjust one or more environmental attributes for the testing unit 2110. Examples of environmental attributes include temperature, noise, pressure, light level, light temperature, orientation, space within the testing unit 2110 (e.g., make the area bigger or smaller via one or more actuators), or the like. The environmental sensor 2108 may be configured to measure one or more of the environmental attributes and provide a report of the measurement. It will be understood that fewer or additional entities may be included to achieve a similar result.

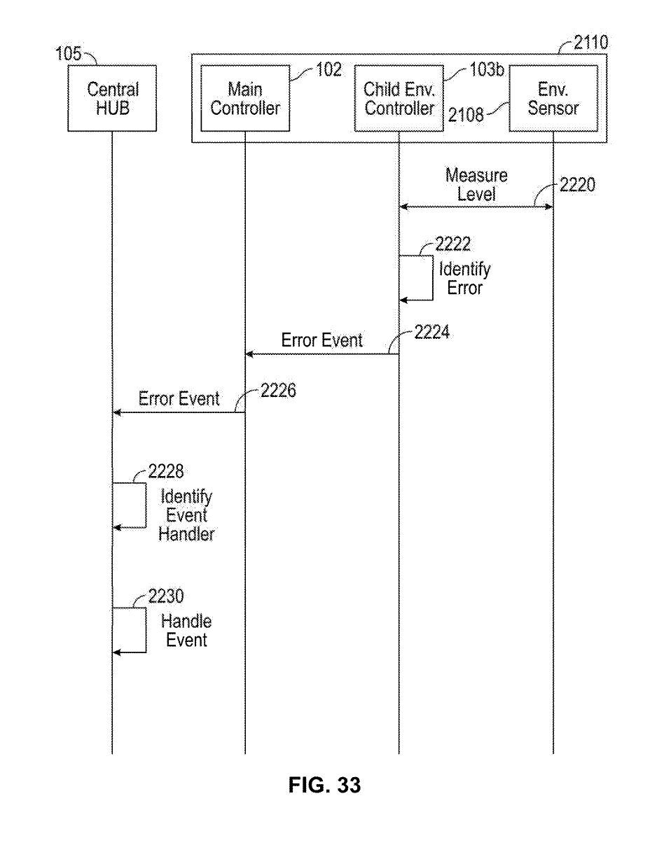

Via message 2120, the central hub 105 may initiate calibration of the testing unit 2110. The message 2120 may be included as a command within a test protocol. The message 2120 may be a scheduled calibration message configured to perform periodic diagnostics on the testing unit 2110. In some implementations, the message 2110 may be transmitted to the testing unit 2110 upon receipt of an error event such as shown in FIG. 30 or below in FIG. 33.

In some implementations, the message 2120 may be a general calibration request. The message 2120 may indicate that the main controller 102 perform calibration for each child controller 103 configured for calibration. In some implementations, the message 2120 may be a specific calibration request indicating a specific environmental attribute to be measured and established (e.g., light, sound, temperature, pressure, size, color).

The main controller 102 may determine which child controller(s) 103 should be activated to achieve the desired calibration level. As shown in FIG. 32, the child environmental controller 103b is identified and provided a message 2122 to configure the child environmental controller 103b to the desired calibration level. The main controller 102 may then exchange messages 2124 with the environmental sensor 2108 to obtain measurements for the environment attribute. The level may not meet the desired calibration level for a variety of reasons. For example, it may be because of degradation in the child environmental controller 103b. For example, a light source may decrease in output capacity after prolonged use. As such, an instruction to set light level to 50 lumens may actually produce a light level at 45 lumens. In such instances, the main controller 102 may request level at 60 lumens to achieve the desired result of 45 lumens. As another example, there may be ambient factors affecting the environmental attribute. For instance, if the testing unit 2110 is located near a window, ambient light may change the level of light within the testing unit 2110. As such, to achieve 50 lumens of light, the lighting controller may need only to produce 45 lumens to augment the additional 5 lumens of ambient light. Messaging 2122 and 2124 may be referred to as testing unit calibration messaging 2130. The testing unit calibration messaging 2130 may be performed multiple times to achieve the desired calibration level. In some implementations, the testing unit calibration messaging 2130 may be configured to be performed a predetermined number of times. If the desired level is not achieved after the predetermined number of attempts to calibrate the environmental attribute, the main controller 102 may be configured to provide an error message, such as to the central hub 105 and/or an operator interface for the testing unit 2110 to indicate a potential error. The measurement data may be stored by the main controller 102 for dynamic adjustment of protocol configuration commands during a test, such as described above.

The main controller 102 may provide a message 2140 including calibration information regarding the desired level. The calibration information may indicate one or more of: the number of attempts to calibrate, whether the calibration was successful, time and/or date information when the calibration was performed, image or audio data such as received by the environmental sensor 2108, an identifier for the testing unit 2110, an identifier for the child environment controller 103b, an identifier for the environmental sensor 2108, and the like.

The central hub 105, via message 2150, may store the calibration information. The central hub 105 may use the stored calibration information when determining which testing unit should be used for a particular test. For example, if a testing unit was unable to be calibrated to a specific temperature, that testing unit may not be selected for executing a test requiring the specific temperature. The stored calibration information may also be used to provide a status of the testing units under control of the central hub 105. For example, if the power required to achieve a requested light level is near the maximum available power. This can allow automatic identification of testing units that may need repair or replacement before they actually malfunction. This can be particularly advantageous in performing controlled studies over a period of time. If a testing unit malfunctions during the test, it may be necessary to disqualify the subject and results for the subject from the study. Furthermore, the stored calibration information may be used to dynamically update commands sent to the main controller 102 to account for identified discrepancies between desired level and actual output level for the environmental attribute.

FIG. 33 shows a message flow diagram of dynamic environmental error detection. The message flow shown in FIG. 33 includes messages exchanged between exemplary entities selected to highlight certain features related to dynamic environmental error detection. It will be understood that fewer or additional entities may be included to achieve a similar result.

FIG. 33 shows similar entities as shown in FIG. 32. Whereas in FIG. 32, the central hub 105 requested calibration, in FIG. 33, a flow is shown whereby the testing unit 2110 self-identifies an error and provides a report of the same to the main controller 102 and ultimately the central hub 105.

The environmental sensor 2108 may provide measurements of an environmental attribute to the child environmental controller 103b. In some implementations, the measurements may be provided to the main controller 102 in addition or in the alternative. The measurements may be provided via message 2220. The measurement may be provided according to a schedule or upon request from an entity within the testing unit 2110 (e.g., the main controller 102 or the child environmental controller 103b or another child controller 103 not shown). The message 2220 may include an identifier for the environmental sensor 2108.

The recipient of the message 2220, via messaging 2222, may identify an error. The error may be identified by comparing a measurement value to an expected value. If the measurement value deviates from the expected value, the error may be identified. For example, the child environmental controller 103b may be a light source. The child environmental controller 103b may be configured with an allowable range of light level. If the measurement level included in a message during the messaging 2222 indicates the level is outside the allowable range, an error may be identified.

Via message 2224 the child environmental controller 103b may provide an indication of the error to the main controller 102. As shown in FIG. 33, the main controller 102 does not handle the error, but rather prepares and transmits a message 2226 indicating the error event to the central hub 105. In some implementations, the main controller 102 may attempt to correct the error prior to notifying the central hub 105.

The central hub 105 via message 2228 may identify an event handler for the error identified by the message 2226. The message 2228 may include querying a data source for the proper handler or chain of handlers for the error. For example, if the error is an excessive temperature error, a handler may be identified to initiate shutting down the testing unit 1110 or activating climate control system at a site where the testing unit 2110 is located (e.g., in a laboratory). Via message 2230, the identified handler is provided the error and the error is handled thereby. It will be appreciated that in some implementations, the handler may be a remote handler. In such instances, the central hub 105 may forward the error to another entity, such as a meta hub 132 or emergency response system, for handling.

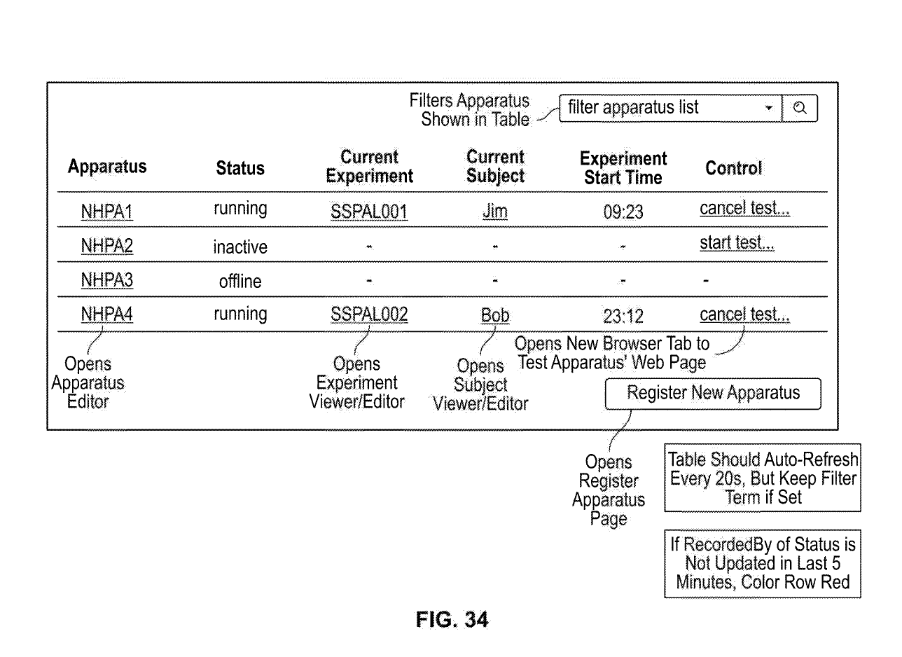

FIG. 34 shows a user interface diagram for a testing system dashboard. The dashboard shown in FIG. 34 provides a listing of all testing units (“apparatus”) registered with the testing system. For each testing unit, a status, a current experiment, a current subject, and experiment start time information is provided. Each testing unit may also be associated with one or more control functions. The control functions may be activated to cause the associated testing unit to perform the indicated function. For example, testing unit NHPA1 includes a control “cancel test.” Activation of this control element causes a command to be sent to the main controller 102 for the testing unit to cancel the active test. Other examples of control elements that may be shown are “start test” to start a test, “calibrate” to initiate calibration of the testing unit, or “shut down” to shut down a testing unit.

Information elements may also be controls to activate additional interface functions. For example, if a current subject is associated with a testing unit, the subject name may be activated to present information about the subject such as weight, height, test results, tests performed, scheduled future tests, and the like. As another example, if an experiment is currently running on a testing unit, the experiment name may be activated to present information about the experiment. This information may include results, streaming video, streaming audio, the protocol configuration used for the experiment, information about a study in which the experiment is included, and the like. The name of the testing unit may also be activated to present information about the testing unit such as a status log over time, calibration information, scheduled tests, maintenance information, registered child controllers 103, registered sensors, and the like. The interface for a testing unit may also provide input fields for editing information about a testing unit.

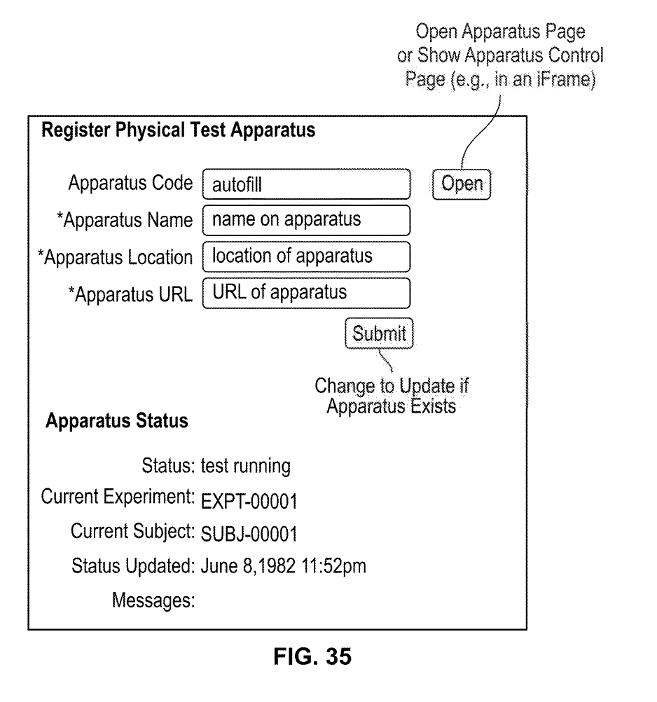

FIG. 35 shows a user interface diagram for testing unit registration and status. The interface shown in FIG. 35 allows registration and/or updating of information identifying a testing unit. The interface may also include status information for the testing unit such as operational status, current experiment, current subject, and last status update. Information provided via the interface shown in FIG. 35 may be controls that may be activated to present additional interface elements. For example, the current experiment or current subject may be activated to provide additional information about the experiment or subject identified.

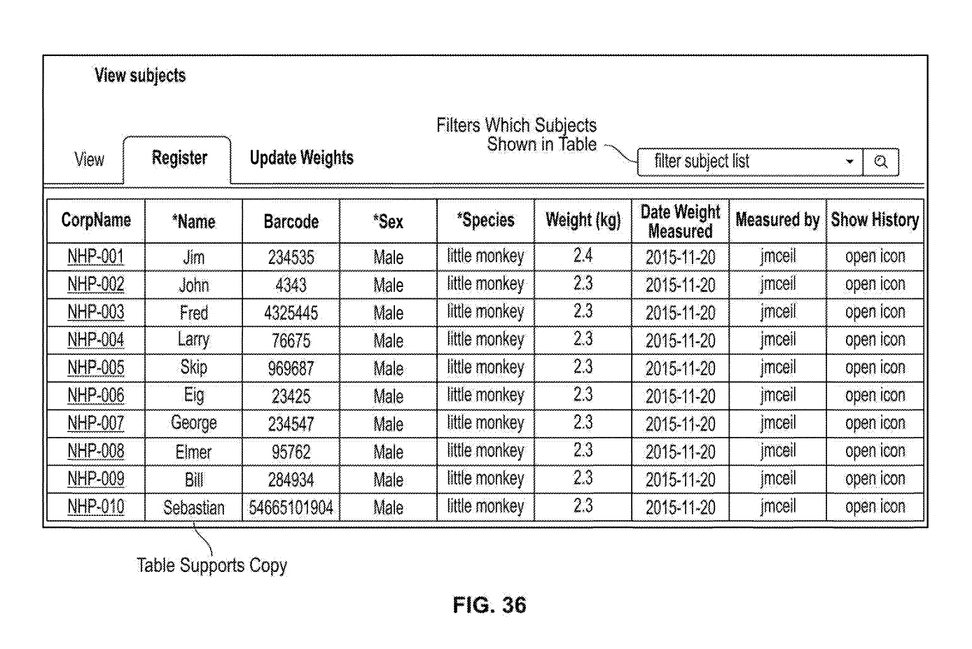

FIG. 36 shows a user interface diagram for viewing test subject information. The interface shown in FIG. 36 provides a table view of subjects registered for testing. A line item may represent information for a specific subject. In some implementations, one or more of the fields may be activated to allow receipt of data about the subject. For example, the weight for a subject may be updated by clicking on the weight cell for the subject. A search/filter function may be included to reduce the number of subjects displayed. When activated from another interface such as those shown in FIG. 24 or 23, the table may show only the identified subject which was activated. The information shown in FIG. 36 may be updated based on results received during a test, such as that shown in FIG. 29.

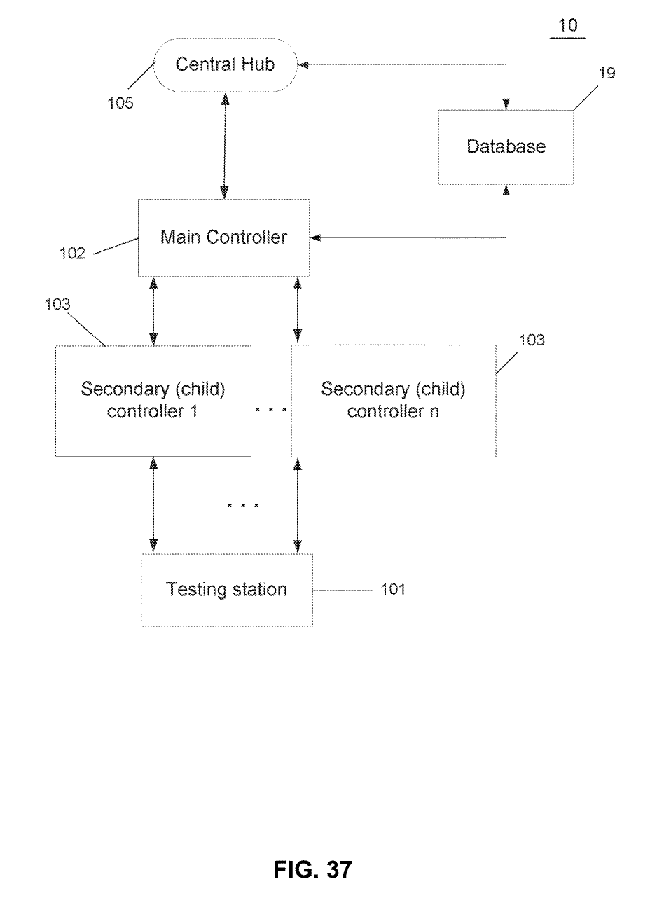

FIG. 37 is a block diagram of a cognitive testing system 10 having a modular architecture according to one embodiment. Depending on the embodiment, certain elements may be removed from or additional elements may be added to the cognitive testing system 10 illustrated in FIG. 37. Furthermore, two or more elements may be combined into a single element, or a single element may be realized as multiple elements. This applies to the remaining embodiments relating to the cognitive testing system.

The cognitive testing system 10 includes a central hub or central hub processor 105, a main controller 102, a plurality of secondary controllers (hereinafter to be interchangeably used with “child controllers”) 103, a database 19 and a testing station 101. The testing station 101 may accommodate a subject (e.g., animal, non-human primate or human) to be tested.

The central hub processor 105 may be located or may run on a separate computer and be configured to communicate data with the main controller 012 over a network. The central hub processor 105 and the main controller 102 may be located or may run on the same computer. The child controllers 103 may include a physical child controller. The physical child controller may be an Arduino microcontroller. The child controllers 103 may include a virtual child controller. The virtual child controller may be located or run on the main controller 012. The virtual child controller may be located or run on a web browser. The web browser may be located or run on the main controller 102. The web browser may be located or run on a separate computer and configured to communicate data with the main controller 102 over a network.

The hardware components of the cognitive testing system 10 can be modular. For example, the cognitive testing system 10 can divide out each function into separate microprocessors, and thus the wiring and schematics of the system 10 can be kept simple. The cognitive testing system 10 can identify faulty components, reducing the time required to troubleshoot.

In some embodiments, the cognitive testing system 10 is made so that all of the components could work independently of each other. Such a system can be far more stable since each function performed by the testing environment can work without interference from other functions. Furthermore, each individual component can be easily replaced or added without affecting any other subsystem. This allows the cognitive testing system 10 to be easily adapted to various different experiments or even entirely different testing environments.

In some embodiments, to make the cognitive testing system 10 modular, the system 10 is divided into various subsystems. The cognitive testing system 10 can be split into a plurality of distinct hierarchical levels. For example, the cognitive testing system 10 is split into three hierarchical levels: the central hub 105, the main controller 102 and the secondary controllers 103.

Each level can abstract lower level functions by interacting with the levels above. For example, the central hub 105 runs software configured to translate experiment protocols into hardware commands. The main controller 102 can subsequently retrieve the commands and assign them to an appropriate secondary controller 103. The assigned secondary controller 103 can interface with the testing station 101 based on the commands received from the main controller 102. For example, when one child controller 103 in charge of dispensing reward pellets is given a dispense command by the central hub 105, the main controller 102 can send a “dispense pellet” command to the pellet dispenser instead of sending an actuation signal to the motor of the testing station 101. The pellet dispenser then handles the interface with the motor and sensor feedback. This makes it easier to change the hardware elements of the system 10 later, because the main controller 102 will not have to change.

The central hub 105 may provide one or more testing commands to the main controller 102. The commands may be computer-readable instructions for the secondary controllers 103 to control the testing station 101. For example, the commands include a pellet dispense command. The central hub 105 can be implemented with a computer that runs software configured to translate experiment protocols into hardware commands that the main controller 102 and the secondary controllers 103 can understand. The central hub 105 may receive the commands from an operator or manager of the cognitive testing system 10. The central hub 105 may include a memory (not shown) that stores the received commands. The central hub 105 may communicate data with the main controller 102 via a variety of communication protocols including, but not limited to, transmission control protocol (TCP). The data may have a JavaScript object notation (JSON) format. For example, the central hub 105 can send a message having a JSON format via TCP.

Although only one central hub 105 is shown on FIG. 37, two or more central hubs can be used depending on the embodiment. Furthermore, although only one main controller 102 is shown on FIG. 37, two or more main controllers can be used depending on the embodiment. Moreover, although multiple child controllers 103 are shown on FIG. 37, only one child controller 103 can be used depending on the embodiment.

The TCP protocol allows a client such as the main controller 102 (or the secondary controller 103) and a server such as the central hub 105 (or the main controller 102) to send and receive streams of data. TCP also provides built-in error checking and redundancy, meaning communications between the client and server can be more reliable. TCP/IP networks also allow for easier testing and simulation as code is readily available due to extensive online documentation. TCP can allow system testing without writing any additional code. For example, the central hub 105 can be simulated by sending commands to the main controller 102 using, for example, a “telnet” command or protocol. Here, telnet is a session layer protocol used on the Internet or local area networks to provide a bidirectional interactive text-oriented communication facility using a virtual terminal connection.

The main controller 102 may receive and parse commands from the central hub 105 and assigns them to an appropriate secondary or child controller 103. For example, when the central hub 105 sends a “dispense” command, the main controller 102 is asked to dispense a pellet, not the pellet dispenser. The main controller 102 may abstract the function of a proper child controller 103 from the commands received from the central hub 105. The main controller 102 can delegate this task to the appropriate child controller 103, the pellet dispenser in this situation. The main controller 102 may also send feedback to the central hub 105 such as touch coordinates, system health checks and status updates.

The main controller 102 can be implemented with a general purpose computer or a special purpose computer. The general purpose or special purpose computer can be, for example, an Intel x86 embedded PC. The main controller 102 can have a configuration based on, for example, i) an advanced RISC machine (ARM) microcontroller and ii) Intel Corporation's microprocessors (e.g., the Pentium family microprocessors). In one embodiment, the main controller 102 is implemented with a variety of computer platforms using a single chip or multichip microprocessors, digital signal processors, embedded microprocessors, microcontrollers, etc. In another embodiment, the main controller 102 is implemented with a wide range of operating systems such as Unix, Linux, Microsoft DOS, Microsoft Windows 7/8/10Vista/2000/9x/ME/XP, Macintosh OS, OS/2, Android, iOS, and the like.

The main controller 102 can be programmed with a high-level programming language such as Python. Python can provide easy multi-platform support and verbosity of language. The main controller 102 can be programmed with Python using only standard libraries. This allows the main controller 102 to work on Linux, Mac, Windows or any other operating system that can interpret Python. Furthermore, Python and all required libraries used by the main controller 102 may come pre-installed with most Linux machines. The main controller 102 may use a separate thread to listen to any incoming TCP requests from the central hub 105. In some embodiments, the main controller 102 requires an Ethernet interface and at least one USB connection, and the main controller 102 can be executed on a Linux machine.

The main controller 102 may communicate data with the child controllers 103 using a communication protocol, including, but not limited to, predefined serial universal asynchronous receiver/transmitter (UART), universal serial bus (USB), controller area network (CAN), RS 485, RS 232 and 10/100 Base T. In some embodiments, when the main controller 102 sends the string “60%” to a child controller, the child controller 103 can understand that it can set either the light or sound level at the testing station 101 to 60% intensity. This standard means that in the future, more child controllers can be easily added if an experiment needs functionality that is currently not provided. As long as the new subsystem follows the standard, the main controller 102 will only need small changes to communicate with the newly added child controller. Furthermore, adding this new child controller would have no effect on any of the other child controllers.

The secondary controllers 103 receive commands from the main controller 102 and control the testing station 101 based on the commands. Each of the secondary controllers 103 can be fully independent from each other. The secondary controllers 103 can provide appropriate feedback to the main controller 102. As described above, the secondary controllers 103 can communicate data with the main controller 102 using a common UART serial protocol. At least one of the secondary controllers 103 (e.g., video and display controllers to be discussed later) can be implemented with a general or special purpose computer such as a regular Intel x86 computer.

Each of the secondary controllers 103 can control at least one hardware component of the testing station 101 and/or at least one environmental condition in the testing station 101 based at least in part on the operating parameter. The at least one hardware component can include, but is not limited to, an input device, an output device, a data processing device and a pellet or reward dispensing device of the testing station 101. The at least one environmental condition can include, but is not limited to, temperature, humidity, light (e.g., brightness) or sound (e.g., noise level) in the testing station 101.

At least one of the secondary controllers 103 can be implemented with a microcontroller. The microcontroller can be a USB based microcontroller such as an Arduino microcontroller. The Arduino microcontroller uses USB serial communication, allowing it to interface with any main controller 102. The Arduino microcontroller can perform a variety of functions while still being common and easy to program. In some embodiments, by using Arduinos for most child controllers 103, the entire testing environment can be greatly simplified while still allowing for highly specialized child controllers 103.

There can be two types of child controllers 103: physical controllers and logical controllers. At least one of the physical child controllers can be a USB based Arduinos controller connected, via printed circuit boards (PCBs), to the hardware of the cognitive testing system 10 (e.g., the main controller 102). The pellet dispenser (to be described in detail later) is an example of the physical child controller. The logical controller can include a display controller, which handles, for example, touchscreen inputs to the cognitive testing system 10 and a video controller, which handles, for example, the video streams in the system 10. The logical child controllers can be implemented on the same PC or the same mother board as the main controller 102, but in separate Python classes. The functions of each of the logical child controllers can be handled by the classes themselves, meaning that the main controller 102 simply makes function calls to interact with the logical child controllers. This can achieve the design philosophy of modularity since the main controller 102 passes the actual functionality to a distinct logical child controller.

A single child controller can be split into two or more child controllers 103. In some embodiments, a single child controller (e.g., sound controller) is split into two controllers: a noise controller and a tone controller. In some embodiments, due to the modular design of the system 10, the only changes required in the main controller 102 are to modify the list of child controllers and their corresponding functions. The modular design allows the new child controller to be implemented without major changes to the system 10.

The database 19 can store various types of information used to perform the cognitive testing. For example, the database 19 can store Python libraries to handle JSON. Commands and responses sent between the central hub 105 and the main controller 102 can be encapsulated into a JSON format. The data having the JSON format can be stored in the database 19. The database 19 can also store files required to use the communication protocols.

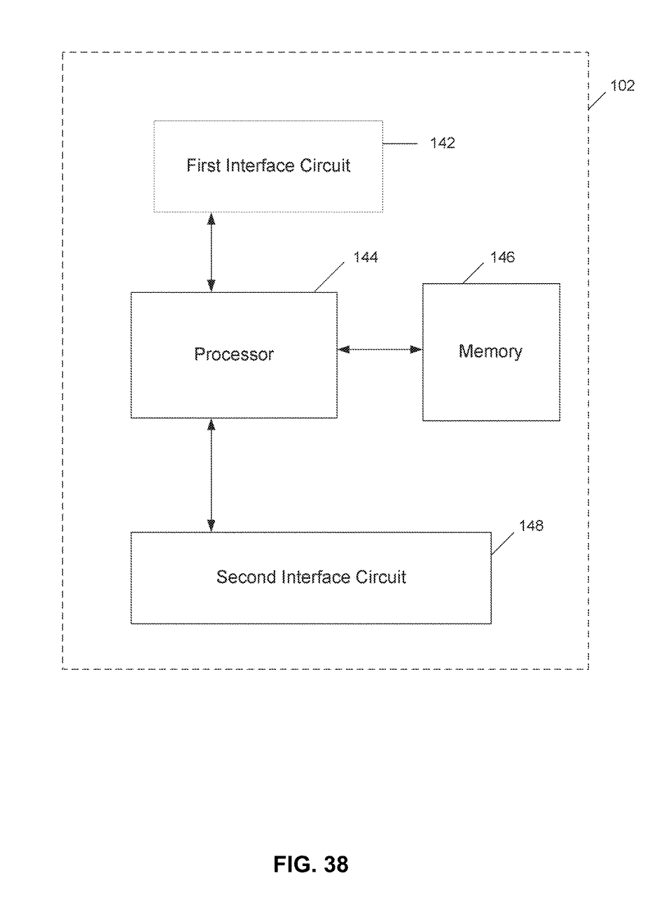

FIG. 38 is a block diagram of the main controller 102 of FIG. 37 according to one embodiment. Depending on the embodiment, certain elements may be removed from or additional elements may be added to the main controller 102 illustrated in FIG. 38. Furthermore, two or more elements may be combined into a single element, or a single element may be realized as multiple elements. This applies to the remaining embodiments relating to the main controller.

The main controller 102 includes a first interface circuit 142, a processor 144, a memory 146 and a second interface circuit 148. The first interface circuit 142 can interface data communication between the central hub 105 and the processor 144 of the main controller 102. The first interface circuit 142 can be implemented with a variety of interface circuits that allows the central hub 105 and the processor 144 to communicate data with each other via a variety of communication protocols including, but not limited to, TCP.

The second interface circuit 148 can interface data communication between the processor 144 of the main controller 102 and the child controllers 103. The second interface circuit 148 can be implemented with a variety of interface circuits that allow the processor 144 and the child controllers 103 to communicate data with each other via a variety of communication protocols such as UART, USB, CAN, RS 485, RS 232 and/or 10/100 Base T.

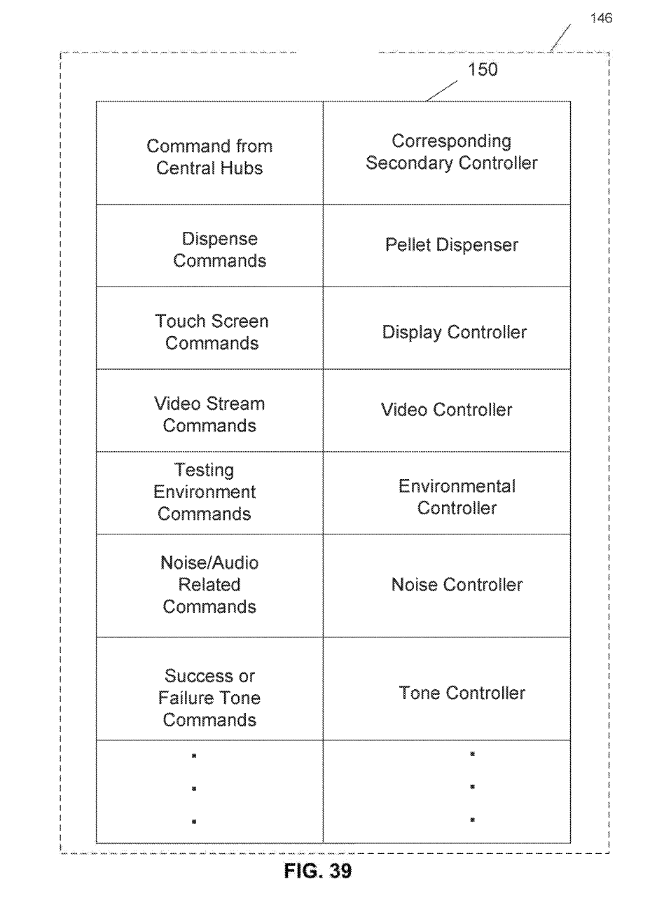

The memory 146 can store various types of information used to perform the function of the main controller 102. For example, as shown in FIG. 39, the memory 146 can store a look-up table 150 that matches commands received from the central hub 105 with the corresponding secondary controllers.

For example, dispense commands correspond to the pellet dispenser (hereinafter to be interchangeably used with a pellet controller). In some embodiments, the memory 146 allows the main controller 102, which receives the dispense commands from the central hub 105, to determine the corresponding secondary controller, here, the pellet dispenser.

Touch screen commands and video stream commands respectively correspond to the display controller and the video controller. In some embodiments, the memory 146 allows the main controller 102, which receives the touch screen commands from the central hub 105, to determine the corresponding secondary controller (i.e., the display controller). The memory 146 can also allow the main controller 102, which receives the video stream commands from the central hub 105, to determine the corresponding secondary controller (i.e., the video controller).

Similarly, testing environment commands, noise/audio related commands, and success/failure commands respectively correspond to the environmental controller, the noise controller and the tone controller. In some embodiments, the memory 146 allows the main controller 102, which receives the testing environment commands from the central hub 105, to determine the corresponding secondary controller (i.e., the environmental controller). The memory 146 can also allow the main controller 102, which receives the noise/audio related commands from the central hub 105, to determine the corresponding secondary controller (i.e., the noise controller). Furthermore, the memory 146 can allow the main controller 102, which receives the success/failure commands from the central hub 105, to determine the corresponding secondary controller (i.e., the tone controller).

Although only six commands and six corresponding secondary controllers are listed in the look-up table 150, additional commands and corresponding secondary controllers can be subsequently added. Furthermore, less than six commands and corresponding secondary controllers can be listed in the look-up table 150. In some embodiments, the memory 146 can allow the main controller 102, which receives two or more of the above commands from the central hub 105, to concurrently or sequentially determine the corresponding secondary controllers.

In other embodiments, the processor 144 can determine the corresponding secondary controllers without considering the look-up table 150. For example, commands from the central hub 105 are written such that the processor 144 can understand without referring to other information. In these embodiments, the look-up table 150 can be omitted.

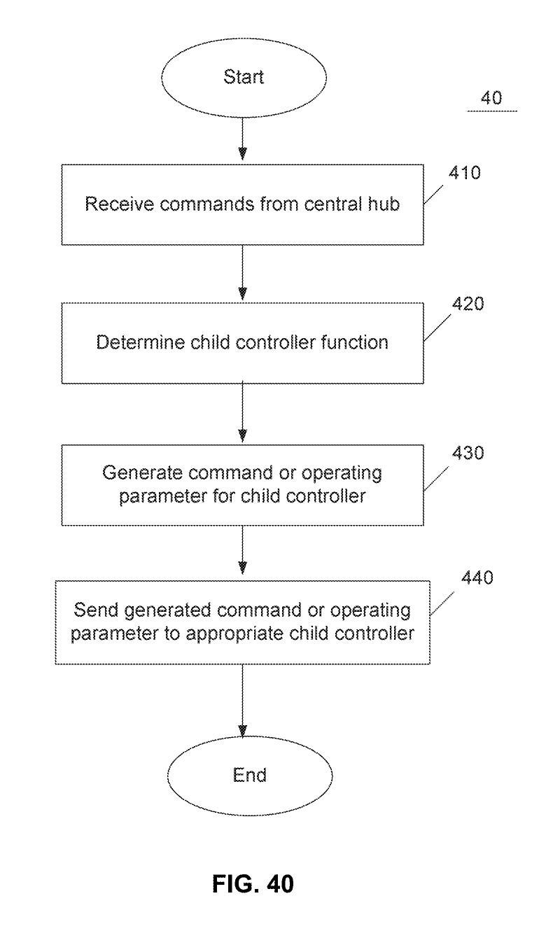

The processor 144 can receive commands from the central hub 105 and determine an appropriate secondary controller based on the information stored on the memory 146. The processor 144 can be implemented with a variety of processors discussed above with respect to the main controller 102. The operations of the processor 144 will be described in greater detail with reference to FIG. 40.

FIG. 40 is a flowchart for an example cognitive testing operation or procedure 40 of the processor 144 of FIG. 38 according to one embodiment. In some embodiments, the procedure 40 (or at least part of the procedure) is implemented in a conventional programming language, such as C or C++ or another suitable programming language. In some embodiments, the program is stored on a computer accessible storage medium of the main controller 102, for example, the memory 146. In other embodiments, the program is stored on a computer accessible storage medium of at least one of the central hub 105 and the secondary controllers 103. In other embodiments, the program is stored in a separate storage medium. The storage medium may include any of a variety of technologies for storing information. In one embodiment, the storage medium includes a random access memory (RAM), hard disks, floppy disks, digital video devices, compact discs, video discs, and/or other optical storage mediums, etc. In another embodiment, the processor 144 is configured to or programmed to perform at least part of the above procedure 40. In another embodiment, at least part of the procedure 40 can be implemented with embedded software. Depending on the embodiment, additional states may be added, others removed, or the order of the states changed in FIG. 40. The description of this paragraph applies to the procedures of FIGS. 5, 15, 23, 24 and 26. Referring to FIG. 40, an example operation of the processor 144 of the main controller 102 will be described.

In state 410, the processor 144 receives one or more testing commands from the central hub 105. For example, the processor 144 receives the testing commands from the central hub 105 via the first interface circuit 142. The procedure 40 can additionally include setting up communication (e.g., TCP connection) between the central hub 105 and the main controller 102, between the main controller 102 and the secondary controllers 103, and/or between the central hub 105 and the secondary controllers 103. The procedure 40 can further include forwarding commands from the central hub 105 to the appropriate secondary controller when the communication is established.

The procedure 40 can further include detecting the secondary controllers 103 by scanning the serial ports of the main controller 102. For example, the memory 146 of the main controller 102 can store all serial devices. For each device found, the processor 144 can first attempt to read the serial buffer to make sure that it is empty. Then the processor 144 can send the command “i” to identify the child controller type. The memory 146 of the main controller 102 can store the identification information in a dictionary attribute as an open serial connection to each child controller. The processor 144 can offer an application-programming interface for other python modules to interact with the child controller 103 by abstracting the serial communications and commands. Commands to activate a device on a child controller 103 are function calls like “playTone( )” or “dispensePellet( )” The processor 144 can deal with the communications to simplify interactions with child controllers 103 and make the code more readable. This Python class can contain constants to represent serial commands sent to the child controllers 103. To know which ASCII character corresponds to a particular command, the processor 144 can look up this information among the constants of the Python class.

In state 420, the processor 144 determines the child controller function corresponding to the received testing commands. For example, when the received command is a “dispense” command, the processor 144 determines the child controller function to be a pellet dispenser.

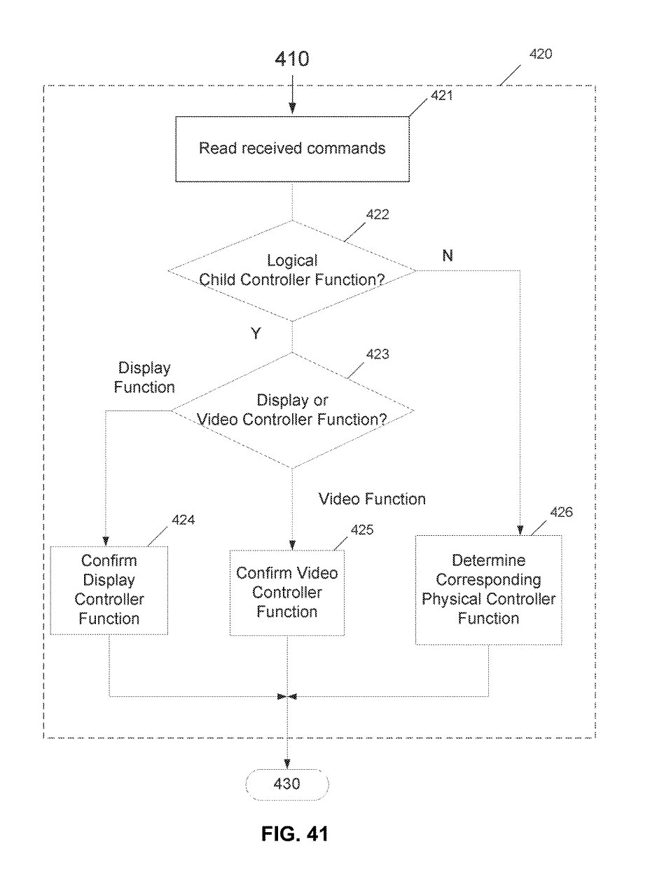

FIG. 41 shows an example procedure of the determining state (420) according to some embodiments. As shown in FIG. 41, state 420 includes at least some of states 421-426. In state 421, the processor 144 reads the command received from the central hub 102. In state 422, the processor 144 determines whether the received command relates to logical controller function. The physical child controllers (e.g., tone/noise/environment controllers and pellet dispenser) can be USB connected to the main controller 102. The logical child controllers (e.g., display/video controllers) can be implemented on the same mother board as the main controller 102. If it is determined in state 422 that the received command does not relate to logical controller function, the processor 144 determines that it relates to physical controller function (state 426).

If it is determined in state 422 that the received command relates to logical controller function, the processor 144 determines whether the received command relates to display controller function or video controller function (state 423). If it is determined in state 423 that the logical controller function is a display controller function, the processor 144 confirms the display controller function and moves to state 430 (state 424). If it is determined in state 423 that the logical controller function is a video controller function, the processor 144 confirms the video controller function and moves to state 430 (state 425).

In other embodiments, the procedure 420 can be modified such that the processor 144 determines in state 422 whether the received command relates to a physical controller function (instead of a logical controller function) and proceeds accordingly thereafter.

In the above description, the protocols were discussed as experimental test protocols. The disclosed features may be used to implement other protocols such as treatment or educational protocols without departing from the spirit of the control and execution features.

Returning to FIG. 40, in state 430, the processor 144 generates a command or operating parameter for the determined child controller. The processor 144 can generate the command using a communication protocol that the child controller can understand and act based on the command. The communication protocol can be UART. For example, as discussed above, the string “60%” command or operating parameter can be understood by the child controller to set either the light or sound level at the testing station 101 to 60% intensity. In state 440, the processor 144 sends the generated command to the appropriate child controller. In some embodiments, the memory 146 can store information that matches the commands received from the central hub 105 and the command to be sent to the child controllers 103. In these embodiments, the processor 144 can retrieve the corresponding command from the memory 146 and transmit the retrieved command to the corresponding child controller 103.

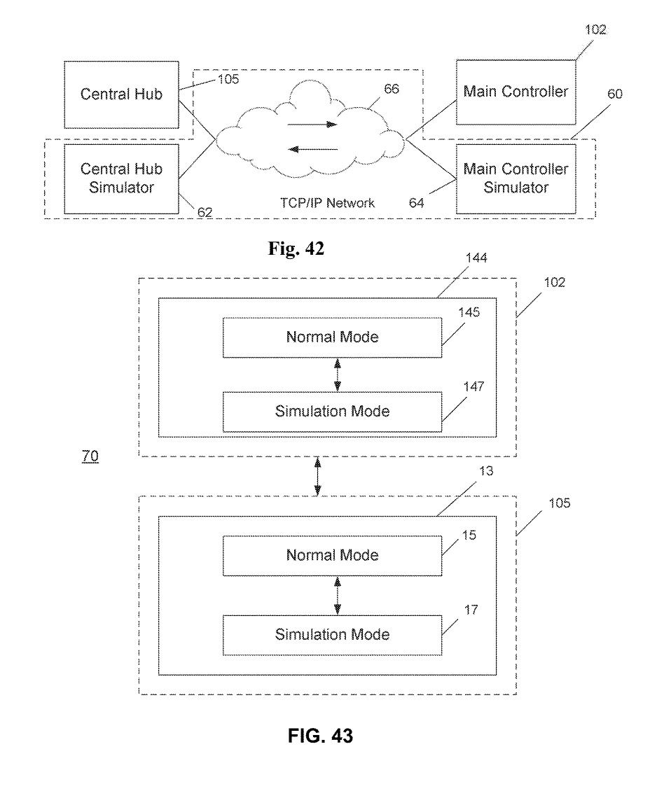

FIG. 42 illustrates an example cognitive testing simulation system 60 for simulating the central hub 105 and the main controller 102 according to some embodiments. Such a system can be useful, for example, so that hardware and software engineers, as well as maintenance and test staff, can develop or exercise functions of part of the system without the other system being functional or even present. The simulation system 60 includes a central hub simulator 62, a main controller simulator 64 and a network 66. The simulation system 60 is electrically connected to the central hub 105 and the main controller 102. The electrical connection can be wired or wireless. The central hub simulator 62 can simulate or test the central hub 105 to determine whether the element is working properly. The main controller simulator 64 can simulate or test the main controller 102 to determine whether the element is working properly. For example, each of the central hub simulator 62 and the main controller simulator 64 can respond to known commands with known responses. As another example, the central hub simulator 62 can control the central hub 105 to send testing commands to the main controller 102 and determine how the central hub 105 interacts with the main controller 102. Furthermore, the main controller simulator 64 can control the main controller 102 to send a command or operating parameter to the corresponding secondary controller 103 and determine how the main controller 102 interacts with the secondary controller 103. The central hub simulator 62 and the main controller simulator 64 can independently simulate or test the central hub 105 and the main controller 102 from each other.

FIG. 43 illustrates an example cognitive testing simulation system 70 for internally simulating the central hub 105 and the main controller 102 according to one embodiment. The internal cognitive testing simulation system 70 can include the central hub 105 and the main controller 102. The central hub 105 includes a processor 13. The processor 13 can operate between a normal operational mode 15 and a simulation mode 17. The processor 13 can be switched between the two modes 15 and 17 via a hardware or software switch (not shown). The processor 144 of the main controller 102 can operate between a normal operational mode 145 and a simulation mode 147. In the simulation modes 147 and 17, each of the processors 144 and 13 can perform the simulation operation discussed above with respect FIG. 42. The processor 144 can be switched between the two modes 145 and 147 via a hardware or software switch (not shown). Upon being switched to the simulation mode, the processor 144 can run automatically in a resizable window on any computer platform.

The various operations of methods described above may be performed by any suitable means capable of performing the operations, such as various hardware and/or software component(s), circuits, and/or module(s). Generally, any operations illustrated in the figures may be performed by corresponding functional means capable of performing the operations.

The various illustrative logical blocks, modules and circuits described in connection with the present disclosure may be implemented or performed with a general purpose processor, a graphics processor unit (GPU), a digital signal processor (DSP), an application specific integrated circuit (ASIC), a field programmable gate array (FPGA) or other programmable logic device (PLD), discrete gate or transistor logic, discrete hardware components or any combination thereof designed to perform the functions described herein.

The systems and methods described herein may be implemented on a variety of different computing devices. They may use general purpose or special purpose computing system environments or configurations. Examples of computing systems, environments, and/or configurations that may be suitable for use with the invention include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like.

A general purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In one or more aspects, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A stable storage media may be any available media that can be accessed by a computer.

By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, include compact disc (CD), laser disc, optical disc, digital versatile disc (DVD), floppy disk, and Blu-ray® disc where disks usually reproduce data magnetically, while discs reproduce data optically with lasers.

### Sounds

Both the light and sound levels may be properly calibrated and detected to ensure that the testing environment is reproducible between units and overtime. This allows the system to be set to a precise sound and light level every time. In some aspects, a noise controller 103d is configured to play white noise to prevent external interference with the test. A tone controller 103c may be configured to play tones that currently signify whether a test subject got an answer right or wrong.

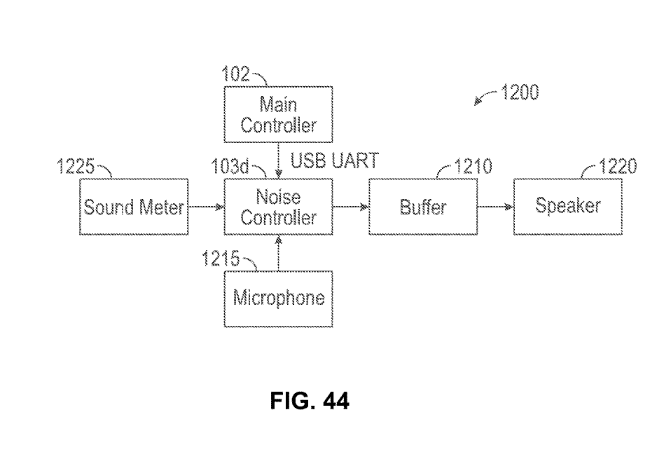

FIG. 44 illustrates an exemplary arrangement for a noise controller speaker 1220, microphone 1215, and sound meter 1225. As shown, the location of the microphone 1215 close to the speaker 1220 allows the microphone 1215 to have maximum sensitivity to the volume of the white noise. In certain embodiments, the sound meter 1225 is positioned such that its data will closely approximate the experience of the test subject. As a result, the sound decibel value given by the meter 1225 is an estimate of the sound level heard by the test subject. In some aspects, the meter 1225 may also be positioned such that it is close enough to the environment camera track the video stream.

In some aspects, the speaker 1220 is configured to play a sound corresponding to the voltage signal it receives. The sound level meter 1225 and/or the microphone 1215 may be configured to track the sound waveform inside the testing environment and produce a voltage corresponding to the noise level.

In some embodiments, animal testing includes cognitive testing. As described herein, cognitive testing can be implemented in many different ways with the systems, apparatuses, devices, and methods embodied in the present disclosure. In any of these embodiments, the performance of a test subject can be compared with that of an appropriate control animal that is the same species as, and otherwise comparable to the subject except with respect to the variable being tested.

In some embodiments, cognitive testing is used to measure or assess a cognitive or motor function in a subject. Neuropsychological assessment, for example, has been used by cognitive psychologists for more than 50 years (Lezak et al. 2004, Neuropsychological Assessment, 4th Edition (New York, Oxford University Press). Tests exist to quantify performance in various functionally distinctive cognitive domains, such as orientation and attention; visual, auditory, or tactile perception; verbal, visual, or tactile memory; remote memory, paired memory; verbal skills; and executive functions. Responses to these tests can be used to determine a score. Individual performance can be evaluated against data to determine extreme (high or low) scores.

Cognitive testing can target an isolated cognitive (or motor) function or multiple functions concurrently. In some embodiments, the present disclosure can include programs that collect and analyze performance data that is generated during implementation of the assays.

In some embodiments, cognitive testing is used to diagnose or identify various aspects of cognitive function brought about by heredity, disease, injury, or age.

In some embodiments, cognitive testing is used to measure a change in a cognitive or motor function in a subject undergoing therapy or treatment of a neurological disorder. The cognitive test can also be directed towards a specific impairment, such as cognitive deficit or motor deficit of the patient. Testing can determine whether treatment can be helpful, the type of treatment to be provided (e.g., the type and dosage of any augmenting agent, as described herein, the type of training, the duration of training, as well as the length and type of ongoing treatment.)

In some embodiments, the assays are used in drug screening, including the action of a candidate drug in enhancing a cognitive or motor function, as discussed further below.

Accordingly, the present disclosure provides improved systems, apparatuses, and methods for cognitive testing. In any of these uses, the modular nature of the systems and methods, along with other features, allows for more rapid development, optimization, customization, modification, and implementation of such testing.

### Training

In some embodiments, cognitive testing can include training—with or without co-administration of a drug. As used herein, the term “training” is interchangeable with “training protocol,” and includes “cognitive training,” “motor training,” and “brain exercises.” Training protocols are used to enhance a cognitive or motor function.

Training protocols can include one or multiple training sessions and are customized to produce an improvement in performance of the cognitive task of interest. For example, if an improvement in language acquisition is desired, training would focus on language acquisition. If an improvement in ability to learn to play a musical instrument is desired, training would focus on learning to play the musical instrument. If an improvement in a particular motor skill is desired, training would focus on acquisition of the particular motor skill. The specific cognitive task of interest is matched with appropriate training.

In embodiments where cognitive training includes multiple training sessions, the sessions can be massed or can be spaced with a rest interval between each session. In some embodiments, an augmenting agent (as described herein) can be administered before, during or after one or more of the training sessions. In a particular embodiment, the augmenting agent is administered before and during each training session.

An emerging notion is that most, if not all, cognitive domains can be functionally rehabilitated through focused brain exercises or “training. This notion derives from the most fundamental property of the brain: its plasticity. Declarative memory is one manifestation of brain plasticity. Rehabilitation after stroke is another example of brain plasticity for implicit (motor) tasks. Buga et al. 2008, Rom. J. Morphol. Embryol. 49, 279-302. More generally, brain exercise as rehabilitation has a long history in animal models Merzenich et al. 1996, Cold Spring. Harb. Symp. Quant. Biol. 61, 1-8. More recently, this approach has been attempted in clinical studies with some success, including rehabilitation of working memory. Duerden and Laverdure-Dupont 2008, J. Neurosci. 28, 8655-8657; Mahncke et al. 2006, Prog. Brain Res. 157, 81-109; Neville and Bavelie 2002, Prog. Brain Res. 138, 177-188; Smith et al. 2009, J. Am. Geriatr. Soc. 57, 594-603; Tallal et al. 1998, Exp. Brain Res. 123, 210-219; Jaeggi et al. 2008, Proc. Natl. Acad. Sci. USA 105, 6829-6833.

Cognitive domains (or functions) that can be targeted by training protocols include, but are not limited to, the following: attention (e.g., sustained attention, divided attention, selective attention, processing speed); executive function (e.g., planning, decision, and working memory); learning and memory (e.g., immediate memory; recent memory, including free recall, cued recall, and recognition memory; and long-term memory, which itself can be divided into explicit memory (declarative memory) memory, such as episodic, semantic, and autobiographical memory, and into implicit memory (procedural memory)); language (e.g., expressive language, including naming, word recall, fluency, grammar, and syntax; and receptive language); perceptual-motor functions (e.g., abilities encompassed under visual perception, visio-constructional, perceptual-motor praxis, and gnosis); and social cognition (e.g., recognition of emotions, theory of mind).

In specific embodiments, the cognitive function is learning and memory, and more particularly, long term memory.

Similarly, motor domains (or functions) that can be targeted by training protocols include, but are not limited to, those involved in gross body control, coordination, posture, and balance; bilateral coordination; upper and lower limb coordination; muscle strength and agility; locomotion and movement; motor planning and integration; manual coordination and dexterity; gross and fine motor skills; and eye-hand coordination.

Accordingly, cognitive training protocols can be directed to numerous cognitive domains, including memory, concentration and attention, perception, learning, planning, sequencing, and judgment. Likewise, motor training protocols can be directed to numerous motor domains, such as the rehabilitation of arm or leg function after a stroke or head injury. One or more protocols (or modules) underling a cognitive training program or motor training program can be provided to a subject.

Training protocols (or “modules”) typically comprise a set of distinct exercises that can be process-specific or skill-based: See, e.g., Kim et al., J. Phys. Ther. Sci. 2014, 26, 1-6, Allen et al., Parkinsons Dis. 2012, 2012, 1-15; Jaeggi et al., Proc. Natl. Acad. Sci. USA 2011, 108, 10081-10086; Chein et al., Psychon. Bull. Rev. 2010, 17, 193-199; Klingberg, Trends Cogn. Sci. 2010, 14, 317-324; Owen et al., Nature 2010, 465, 775-778; Tsao et al., J. Pain 2010, 11, 1120-1128. Process-specific training focuses on improving a particular domain such as attention, memory, language, executive function, or motor function. Here the goal of training is to obtain a general improvement that transfers from the trained activities to untrained activities based on the same cognitive or motor function or domain. For example, an auditory cognitive training protocol can be used to treat a subject with impaired auditory attention after suffering from a stroke. At the end of training, the subject should show a general improvement in auditory attention, manifested by an increased ability to attend to and concentrate on verbal information. Skill-based training is aimed at improving performance of a particular activity or ability, such as learning a new language, improving memory, or learning a fine motor skill. The different exercises within such a protocol will focus on core components within one or more domains underlying the skill. Modules for increasing memory, for example, may include tasks directed to specific domains involved in memory processing, e.g., the recognition and use of fact, and the acquisition and comprehension of explicit knowledge rules.

Cognitive and motor training programs can involve computer games, handheld game devices, and interactive exercises. Cognitive and motor training programs can also employ feedback and adaptive models. Some training systems, for example, use an analog tone as feedback for modifying muscle activity in a region of paralysis, such as facial muscles affected by Bell's palsy. (e.g., Jankel, Arch. Phys. Med. Rehabil. 1978, 59, 240-242.). Other systems employ a feedback-based close loop system to facilitate muscle re-education or to maintain or increase range of motion. (e.g., Stein, Expert Rev. Med. Devices 2009, 6, 15-19.)

Accordingly, the aspects described may include or be included with brain exercises (training protocols) that target distinct cognitive domains. Such protocols can cover multiple facets of cognitive ability, such as motor skills, executive functions, declarative memory, etc.

In one or more embodiments, the present disclosure can include programs that collect and analyze performance data that is generated during implementation of the training protocols.

In some embodiments, training comprises a battery of tasks directed to the neurological function. In some embodiments, the training is part of physical therapy, cognitive therapy, or occupational therapy.

In some embodiments, training protocols are used to evaluate or assess the effect of a candidate drug or agent in enhancing a cognitive or motor skill in a subject.

### Augmented Cognitive Training

In some embodiments, the efficiency of such training protocols can be improved by administering an augmenting agent. An augmenting agent can enhance CREB pathway function, as described, e.g., in U.S. Pat. Nos. 8,153,646, 8,222,243, 8,399,487; 8,455,538, and 9,254,282. More particularly, this method (known as augmented cognitive training or ACT) can decrease the number of training sessions required to improve performance of a cognitive function, relative to the improvement observed by cognitive training alone. See, e.g., U.S. Pat. No. 7,868,015; U.S. Pat. No. 7,947,731; U.S. 2008/0051437. Accordingly, in one aspect, administering an augmenting agent with a training protocol can decrease the amount of training sufficient to improve performance of a neurological function compared with training alone. In another aspect, administering an augmenting agent with a training protocol may increase the level of performance of a neurological function compared to that produced by training alone. The resulting improvement in efficiency of any methods disclosed herein can be manifested in several ways, for example, by enhancing the rate of recovery, or by enhancing the level of recovery. For further descriptions and examples of augmented cognitive (or motor) training and augmenting agents, see, e.g., U.S. Pat. Nos. 8,153,646, 8,222,243, 8,399,487, 8,455,538, 9,254,282, U.S. Published Application Nos. 2014/0275548 and 20150050626, and WO/2016/04463, all of which are incorporated by reference.

In some embodiments, training protocols are used in drug screening, such as evaluating the augmenting action of a candidate augmenting agent in enhancing cognitive function. In a particular aspect, the cognitive function is long-term memory.

In some embodiments, training protocols are used in rehabilitating individuals who have some form and degree of cognitive or motor dysfunction. For example, training protocols are commonly employed in stroke rehabilitation and in age-related memory loss rehabilitation.

Accordingly, the present disclosure provides improved systems, apparatuses, and methods for training protocols. In any of these uses, the modular nature of the systems and methods of the present disclosure, along with other features, allows for more rapid development, optimization, customization, modification, and implementation of such protocols.

In specific embodiments, the systems and methods of the present disclosure are used with augmented training protocols to treat a subject undergoing rehabilitation from a trauma-related disorder. Such protocols can be restorative or remedial, intended to reestablish prior skills and cognitive functions, or they can be focused on delaying or slowing cognitive decline due to neurological disease. Other protocols can be compensatory, providing a means to adapt to a cognitive deficit by enhancing function of related and uninvolved cognitive domains. In other embodiments, the protocols can be used to improve particular skills or cognitive functions in otherwise healthy individuals. For example, a cognitive training program might include modules focused on delaying or preventing cognitive decline that normally accompanies aging; here the program is designed to maintain or improve cognitive health.

### Methods

In some embodiments, the above described system, apparatuses, and methods can be used in methods of assessing, diagnosing, or measuring a cognitive or motor deficit associated with a neurological disorder. They can also be used in methods of assessing the efficacy of a treatment or therapy in treating a cognitive or motor deficit associated with a neurological disorder. A neurological disorder (or condition or disease) is any disorder of the body's nervous system. Neurological disorders can be categorized according to the primary location affected, the primary type of dysfunction involved, or the primary type of cause. The broadest division is between central nervous system (CNS) disorders and peripheral nervous system (PNS) disorders.

In some embodiments, the neurological disorder corresponds to cognitive disorders, which generally reflect problems in cognition, i.e., the processes by which knowledge is acquired, retained and used. In one aspect, cognitive disorders can encompass impairments in executive function, concentration, perception, attention, information processing, learning, memory, or language. In another aspect, a cognitive disorder can encompass impairments in psychomotor learning abilities, which include physical skills, such as movement and coordination; fine motor skills such as the use of precision instruments or tools; and gross motor skills, such as dance, musical, or athletic performance.

In some embodiments, a cognitive impairment is associated with a complex central nervous system (CNS) disorder, condition, or disease. For example, a cognitive impairment can include a deficit in executive control that accompanies autism or mental retardation; a deficit in memory associated with schizophrenia or Parkinson's disease; or a cognitive deficit arising from multiple sclerosis. In the case of multiple sclerosis (MS), for example, about one -half of MS patients will experience problems with cognitive function, such as slowed thinking, decreased concentration, or impaired memory. Such problems typically occur later in the course of MS—although in some cases they can occur much earlier, if not at the onset of disease.

Cognitive impairments can be due to many categories of CNS disorders, including (1) dementias, such as those associated with Alzheimer's disease, Parkinson's disease, and other neurodegenerative disorders; and cognitive disabilities associated with progressive diseases involving the nervous system, such as multiple sclerosis; (2) psychiatric disorders, which include affective (mood) disorders, such as depression and bipolar disorders; psychotic disorders, schizophrenia and delusional disorder; and neurotic and anxiety disorders, such as phobias, panic disorders, obsessive-compulsive disorder, generalized anxiety disorder; eating disorders; and posttraumatic stress disorders; (3) developmental syndromes, genetic conditions, and progressive CNS diseases affecting cognitive function, such as autism spectrum disorders; fetal alcohol spectrum disorders (FASD); Rubinstein-Taybi syndrome; Down syndrome, and other forms of mental retardation; and multiple sclerosis; (4) trauma-dependent losses of cognitive functions, e.g., impairments in memory, language, or motor skills resulting from brain trauma; head trauma (closed and penetrating); head injury; tumors, especially cerebral tumors affecting the thalamic or temporal lobe; cerebrovascular disorders (diseases affecting the blood vessels in the brain), such as stroke, ischemia, hypoxia, and viral infection (e.g., encephalitis); excitotoxicity; and seizures. Such trauma-dependent losses also encompass cognitive impairments resulting from extrinsic agents such as alcohol use, long-term drug use, and neurotoxins, e.g., lead, mercury, carbon monoxide, and certain insecticides. See, e.g., Duncan et al., 2012, Monoamine oxidases in major depressive disorder and alcoholism, Drug Discover. Ther. 6, 112-122; (5) age-associated cognitive deficits, including age-associated memory impairment (AAMI); also referred to herein as age-related memory impairment (AMI)), and deficits affecting patients in early stages of cognitive decline, as in Mild Cognitive Impairment (MCI); and (6) learning, language, or reading disabilities, such as perceptual handicaps, dyslexia, and attention deficit disorders.

Accordingly, the present disclosure can provide a method of treating a cognitive impairment associated with a CNS disorder selected from one or more of the group comprising: dementias, including those associated with neurodegenerative disorders; psychiatric disorders; developmental syndromes, genetic conditions, and progressive CNS diseases and genetic conditions; trauma-dependent losses of cognitive function; age-associated cognitive deficits; and learning, language, or reading disorders.

In some embodiments, the cognitive or motor deficit is associated with a trauma-related disorder. A neurotrauma disorder includes, but is not limited to: (i) vascular diseases due to stroke (e.g., ischemic stroke or hemorrhagic stroke) or ischemia; (ii) microvascular disease arising from diabetes or arthrosclerosis; (3) traumatic brain injury (TBI), which includes penetrating head injuries and closed head injuries; (4) tumors, such as nervous system cancers, including cerebral tumors affecting the thalamic or temporal lobe; (5) hypoxia; (6) viral infection (e.g., encephalitis); (7) excitotoxicity; and (8) seizures. In some embodiments, the neurotrauma disorder is selected from the group consisting of a stroke, a traumatic brain injury (TBI), a head trauma, and a head injury.

In some embodiments, the neurotrauma disorder is stroke. In some embodiments, the protocols can be used to treat, or rehabilitate, cognitive or motor impairments in subjects who have suffered a stroke. In another embodiment, the neurotrauma disorder is TBI. In some embodiments, the protocols can be used to treat, or rehabilitate, cognitive or motor impairments in subjects who have suffered TBI.

As used herein, the term “about” or “approximately” means within an acceptable range for a particular value as determined by one skilled in the art, and may depend in part on how the value is measured or determined, e.g., the limitations of the measurement system or technique. For example, “about” can mean a range of up to 20%, up to 10%, up to 5%, or up to 1% or less on either side of a given value.

It should be understood that any reference to an element herein using a designation such as “first,” “second,” and so forth does not generally limit the quantity or order of those elements. Rather, these designations may be used herein as a convenient method of distinguishing between two or more elements or instances of an element. Thus, a reference to first and second elements does not mean that only two elements may be employed there or that the first element must precede the second element in some manner.

Also, unless stated otherwise a set of elements may comprise one or more elements. In addition, terminology of the form “at least one of: A, B, or C” used in the description or the claims means “A or B or C or any combination of these elements.” As an example, “at least one of: a, b, or c” is intended to cover: a, b, c, a-b, a-c, b-c, and a-b-c. Similarly, a group of items linked with the conjunction “and” should not be read as requiring that each and every one of those items be present in the grouping, but rather should be read as “and/or” unless expressly stated otherwise. Likewise, a group of items linked with the conjunction “or” should not be read as requiring mutual exclusivity among that group, but rather should also be read as “and/or” unless expressly stated otherwise.

As used herein, the singular forms “a,” “an,” and “the” are intended to include the plural forms as well, unless the context clearly indicates otherwise. As used herein, the term “and/or” includes any and all combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Expressions such as “at least one of,” when preceding a list of elements, modify the entire list of elements and do not modify the individual elements of the list.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

As used herein, the terms “provide” or “providing” encompass a wide variety of actions. For example, “providing” may include storing a value in a location for subsequent retrieval, transmitting a value directly to the recipient, transmitting or storing a reference to a value, and the like, or a combination thereof. “Providing” may also include encoding, decoding, encrypting, decrypting, validating, verifying, and the like.

As used herein, the terms “obtain” or “obtaining” encompass a wide variety of actions. For example, “obtaining” may include retrieving, calculating, receiving, requesting, and the like, or a combination thereof. Data obtained may be received automatically or based on manual entry of information. Obtaining may be through an interface such as a graphical user interface.

As used herein, the term “message” encompasses a wide variety of formats for communicating (e.g., transmitting or receiving) information. A message may include a machine readable aggregation of information such as an eXtensible Markup Language (XML) document, fixed field message, comma separated value (CSV), or the like. A message may, in some implementations, include a signal utilized to transmit one or more representations of the information. While recited in the singular, it will be understood that a message may be composed, transmitted, stored, received, etc. in multiple parts.

A “user interface,” “interactive user interface,” “graphical user interface,” “UI” may refer to a web-based interface including data fields for receiving input signals or providing electronic information and/or for providing information to the user in response to any received input signals. A

UI may be implemented in whole or in part using technologies such as HTML, Flash, Java, .net, web services, and rich site summary (RSS). In some implementations, a user interface (UI) may be included in a stand-alone client (for example, thick client, fat client) configured to communicate (e.g., send or receive data) in accordance with one or more of the aspects described.

As used herein, the term “animal” is interchangeable with “subject” and may be a vertebrate, in particular, a mammal, and more particularly, a non-human primate or a human. The term “animal” also includes a laboratory animal in the context of a pre-clinical, screening, or activity experiment. In some embodiments, an animal is a non-human animal, including a non-human mammal or a non-human primate. In some embodiments, the animal is a non-human primate (such as a macaque). In other embodiments, the animal is a non-human mammal (such as a dog, cat, mouse, or rat) or vertebrate generally. In other embodiments, the animal is an invertebrate, for example, a fruit fly. In other embodiments, the animal is a human, for example, and can include a human in a clinical trial, a human undergoing cognitive assessment, or a human undergoing a training protocol to enhance a cognitive (or motor) function or improve a cognitive (or motor) deficit. Thus, as can be readily understood by one of ordinary skill in the art, the methods, apparatuses, and devices of the present disclosure are particularly suited for use with a wide scope of animals, from invertebrates to vertebrates, including non-human primates and humans.

As used herein, the term “application” refers generally to a unit of executable software that implements a certain functionality or theme. The themes of applications vary broadly across any number of disciplines and functions (such as on-demand content management, e-commerce transactions, brokerage transactions, home entertainment, calculator etc.), and one application may have more than one theme. The unit of executable software generally runs in a predetermined environment; for example, the unit could comprise a downloadable Java Xlet™ that runs within the JavaTV™ environment.

As used herein, the terms “client device” and “end user device” include, but are not limited to, set-top boxes (e.g., DSTBs), gateways, personal computers (PCs), and minicomputers, whether desktop, laptop, or otherwise, and mobile devices such as handheld computers, PDAs, personal media devices (PMDs), and smartphones.

In one embodiment, the central hub runs a domain specific language (DSL).

As used herein, the term “computer program” or “software” is meant to include any sequence or human or machine cognizable steps which perform a function. Such program may be rendered in virtually any programming language or environment including, for example, C/C++, Fortran, COBOL, PASCAL, assembly language, markup languages (e.g., HTML, SGML, XML, VoXML), and the like, as well as object-oriented environments such as the Common Object Request Broker Architecture (CORBA), Java™ (including J2ME, Java Beans, etc.), Binary Runtime Environment (e.g., BREW), and the like.

As used herein, the term “display” means any type of device adapted to display information, including without limitation: CRTs, LCDs, TFTs, plasma displays, LEDs, incandescent and fluorescent devices. Display devices may also include less dynamic rendering devices such as, for example, printers, e-ink devices, and the like.

As used herein, the terms “Internet” and “internet” are used interchangeably to refer to inter-networks including, without limitation, the Internet.

As used herein, the term “interface” (1B), as used in connection with a network or bus. refers to any signal, data, or software interface with a component, network or process including, without limitation, those of the Firewire (e.g., FW400, FW800, etc.), USB (e.g., USB 2.0 or 3.0), Ethernet (e.g., 10/100, 10/100/1000 (Gigabit Ethernet), 10-Gig-E, etc.), Thunderbolt, MoCA, Serial ATA (e.g., SATA, e-SATA, SATAII), Ultra-ATA/DMA, Coaxsys (e.g., TVnet™), radio frequency tuner (e.g., in-band or out-of band, cable modem, etc.), Wi-Fi (e.g., 802.11a,b,g,n,v), WiMAX (802.16), PAN (802.15), or IrDA families.

As used herein, the term “server” refers to any computerized component, system or entity regardless of form which is adapted to provide data, files, applications, content, or other services to one or more other devices or entities on a computer network.

As used herein, the term “user interface” refers to, without limitation, any visual, graphical, tactile, audible, sensory, or other means of providing information to and/or receiving information from a user or other entity.

It should be understood that the foregoing description is only illustrative of the invention. Various alternatives and modifications can be devised by those skilled in the art without departing from the invention. For example, the various operations of methods described above may be performed by any suitable means capable of performing the operations, such as various hardware and/or software component(s), circuits, and/or module(s). Generally, any operations illustrated in the figures may be performed by corresponding functional means capable of performing the operations. Accordingly, the present invention is intended to embrace all such alternatives, modifications and variances which fall within the scope of the appended claims.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims. The functions described may be implemented in hardware, software, firmware, or any combination thereof. If implemented in software, the functions may be stored as one or more instructions on a computer-readable medium.

Thus, certain aspects may comprise a computer program product for performing the operations presented herein. For example, such a computer program product may comprise a computer readable medium having instructions stored (and/or encoded) thereon, the instructions being executable by one or more processors to perform the operations described herein. For certain aspects, the computer program product may include packaging material.

In some aspects, the disclosure is directed to one or more of the following numbered embodiments:
1. A system for animal testing, comprising:

an enclosure including a testing chamber into which an animal can be placed;

a modular hardware controller, comprising:

a first and a second interface connector to a first bus, wherein the first and second interfaces are each configured to electrically and physically connect to a child controller circuit board, wherein the child controller circuit board is configured to receive input from or generate output to the testing chamber, and comprises one or more of a reward dispenser controller board, an environmental controller board, a tone controller board, a noise controller board, and a display controller board, each board comprising a hardware board processor, and

a hardware processor configured to communicate with one or more connected hardware board processors over the first bus and to execute a first virtualized child controller, wherein the first virtualized child controller is configured to control a device that receives input from or generates output to the testing chamber, without utilization of a child controller circuit board.
2. The system of embodiment 1, wherein the hardware processor is further configured to receive instructions for performing a test from a separate computer over a socket-based connection.
3. The system of embodiment 1, wherein the child controller circuit board is configured to be removable from the first or second interface connectors.
4. The system of embodiment 1, wherein the environmental controller board is configured to control an indicator light or house lights within the animal enclosure.
5. The system of embodiment 1, wherein the reward dispenser controller board is configured to control a reward dispenser system, the reward dispenser system comprising a reward dispenser, a pick-up area that is accessible from within the enclosure, and a pathway connecting the reward dispenser to the pick-up area.
6. The system of embodiment 5, wherein the reward dispenser system further comprises an indicator light, and an infrared sensor, the infrared sensor configured to detect when a reward is dispensed into the pick-up area.
7. The system of embodiment 5, further comprising a door to the pick-up area, wherein the reward dispenser system further comprises a second infrared sensor configured to detect when the door to the pick-up area has moved.
8. The system of embodiment 5, wherein the reward dispenser system is configured to dispense a food reward to the pick-up area.
9. The system of embodiment 8, wherein the food reward is a pellet, liquid or paste.
10. The system of embodiment 1, wherein the noise controller board is configured to adjust a level of white noise within the enclosure.
11. The system of embodiment 1, wherein the hardware processor is further configured to send a command to the child controller circuit board and receive a response from the child controller circuit board.
12. The system of embodiment 11, wherein the hardware processor is further configured to send the command to the child controller circuit board over a universal serial bus.
13. The system of embodiment 1, further comprising two child controller circuit boards, each of the child controller circuit boards physically and electrically connected to the first and second interface connectors respectively, each of the two child controller circuit boards comprising an equivalent hardware processor.
14. The system of embodiment 1, further comprising at least one sensor disposed within the testing chamber and electronically coupled to a child controller circuit board.
15. The system of embodiment 14, wherein the at least one sensor comprises a sound meter.
16. The system of embodiment 14, wherein the at least one sensor comprises a thermometer.
17. The system of embodiment 1, wherein the tone controller board comprises a tone emitter and a microphone.
18. The system of embodiment 1, wherein the environmental controller board is configured to control a temperature of the enclosure.
19. The system of embodiment 1, wherein the one or more child controller circuit boards are configured to execute a testing command from the modular hardware controller, the testing command setting one or more of a light, a sound, a temperature, or a humidity condition within the testing chamber.
20. The system of embodiment 1, further comprising a touch screen positioned within the testing chamber, wherein the display controller board is configured to receive input from the touch screen and provide the input to the modular hardware controller.
21. The system of embodiment 20, wherein the display controller board is configured to receive the input from a mouse, pointer, joystick, trackball, or similar device.
22. The system of embodiment 1, further comprising an eye tracking device, wherein one of the child controller boards is configured to receive input from the eye tracking device and provide the input to the modular hardware controller.
23. The system of embodiment 1, further comprising an electroencephalography measuring device positioned within the testing chamber, wherein one of the child controller circuit boards is configured to receive input from the electroencephalography measuring device and provide the input to the modular hardware controller.
24. The system of embodiment 1, further comprising a force measuring device positioned within the testing chamber, wherein one of the child controller circuit boards is configured to receive input from the force measuring device and provide the input to the modular hardware controller.
25. The system of embodiment 1, further comprising a blood pressure measuring device positioned within the testing chamber, wherein one of the child controller circuit boards is configured to receive input from the blood pressure measuring device and provide the input to the modular hardware controller.
26. The system of embodiment 1, further comprising an electronic display device and a touch device within the testing chamber, wherein the one or more child controller circuit boards include a child controller circuit board that is configured to execute test commands by generating an image on the display device and a child controller circuit board configured to receive input from the touch device.
27. The system of embodiment 1, wherein the one or more child controller circuit boards are configured to asynchronously provide data to the modular hardware controller without a request for such information from the modular hardware controller.
28. The system of embodiment 27, wherein the data provided asynchronously comprises one or more of a notification event, an environmental measurement, status configuration and warning.
29. The system of any of the preceding embodiments, wherein the hardware processor is further configured to execute a first virtualized child controller, wherein the first virtualized child controller is configured to control a device that receives input from or generates output to the testing chamber, without utilization of a child controller board.
30. The system of any of the preceding embodiments, wherein the hardware processor is further configured to execute a second virtualized child controller wherein the first virtualized child controller is configured to generate a visual stimulus via a display device positioned within the testing chamber, and the second virtualized child controller is configured to receive input from a touch device positioned within the testing chamber.
31. The system of any of the preceding embodiments, wherein the hardware processor is further configured to communicate with the first virtualized child controller and the at least one child controller board using the same messaging interface.
32. The system of any of the preceding embodiments, wherein the hardware processor is further configured to execute a web browser, and wherein the first virtualized child controller is configured to execute within the web browser.
33. The system of any of the preceding embodiments, further comprising a second hardware processor, wherein the second hardware processor is further configured to execute a first virtualized child controller, wherein the first virtualized child controller is configured to control a device that receives input from or generates output to the testing chamber, without utilization of a child controller board.
34. The system of embodiment 33, wherein the second hardware processor is further configured to execute a second virtualized child controller wherein the first virtualized child controller is configured to generate a visual stimulus via a display device positioned within the testing chamber, and the second virtualized child controller is configured to receive input from a touch device positioned within the testing chamber.
35. The system of embodiment 33, wherein the second hardware processor is further configured to communicate with the first virtualized child controller and the at least one child controller board using the same messaging interface.
36. The system of embodiment 33, wherein the second hardware processor is further configured to execute a web browser, and wherein the first virtualized child controller is configured to execute within the web browser.
37. The system of embodiment 36, wherein the hardware processor and the second hardware processor are in separate computers, and configured to communicate with each other across a network.
38. A modular testing apparatus, comprising:

a means for enclosing an animal under test;

a means for controlling an environment of the means for enclosing, the environment comprising a plurality of aspects, the means for controlling comprising first and second means for electrically and physically connecting to the means for controlling; and

a removable means for controlling at least one of the aspects of the environment of the means for enclosing via either the first or second means for electrically and physically connecting to the means for controlling.
39. The apparatus of embodiment 38, further comprising second removable means for controlling the environment of the means for enclosing.
40. The apparatus of embodiment 38, wherein the removable means for controlling comprises one or more of a reward dispenser control circuit board, an environmental controller control circuit board, a tone controller circuit board, and a noise controller circuit board, each of the circuit boards comprising a hardware board processor.
41. The apparatus of embodiment 38, wherein the at least one aspect of the environment includes a temperature, a humidity, a noise level, a dispensation of a reward, an electronic display, and an acknowledgment of an answer.
42. The apparatus of embodiment 38, further comprising central controlling means, the central controlling means configured to control a plurality of modular testing apparatus.
43. The apparatus of embodiment 38, wherein the animal testing is limited to human testing.
44. The apparatus of embodiment 38, wherein the animal testing is limited to non-human testing.
45. The apparatus of embodiment 38, wherein animal testing includes cognitive testing.
46. The apparatus of embodiment 45, wherein the cognitive testing is used to measure a cognitive or motor function in a subject.
47. The apparatus of embodiment 45, wherein the cognitive testing is used to measure a change in a cognitive or motor function in a subject brought about by heredity, disease, injury, or age.
48. The apparatus of embodiment 45, wherein the cognitive testing is used to measure a change in a cognitive or motor function in a subject undergoing therapy or treatment of a neurological disorder.
49. The apparatus of embodiment 45, wherein cognitive testing includes a training protocol.
50. The apparatus of embodiment 49, wherein the training protocol comprises cognitive training.
51. The apparatus of embodiment 49, wherein the training protocol comprises motor training.
52. The apparatus of embodiment 49, wherein the training protocol comprises process specific tasks.
53. The apparatus of embodiment 49, wherein the training protocol comprises skill based tasks.
54. The apparatus of embodiment 49, wherein the training protocol is for use in enhancing a cognitive or motor function.
55. The apparatus of embodiment 49, wherein the training protocol is for use in rehabilitating a cognitive or motor deficit associated with a neurological disorder.
56. The apparatus of embodiment 55, wherein the cognitive deficit is a deficit in memory formation.
57. The apparatus of embodiment 56, wherein the deficit in memory formation is a deficit in long term memory formation.
58. The apparatus of embodiment 55, wherein the neurological disorder is a neurotrauma.
59. The apparatus of embodiment 58, wherein the neurotrauma is stroke or traumatic brain injury.
60. The apparatus of embodiment 49, wherein the training protocol is an augmented training protocol and further comprises administering an augmenting agent in conjunction with training.
61. A device for control of a plurality of animal tests, comprising:

an electronic hardware processor configured to be in communication with a plurality of main controllers, the processor configured to control, via the communication, a plurality of independent environments within a corresponding plurality of animal enclosures.
62. The device of embodiment 61, wherein the electronic hardware processor is further configured to receive input from the plurality of independent environments via the communication.
63. The device of embodiment 61, wherein the electronic hardware processor is further configured to receive touch screen input from each of the plurality of independent environments.
64. The device of embodiment 61, wherein the electronic hardware processor is further configured to set one or more of a temperature, a humidity, and a noise level within each of the independent environments.
65. The device of embodiment 61, wherein the electronic hardware processor is further configured to dispense a reward to each of the independent environments based on corresponding input received from each of the independent environments.
66. The device of embodiment 61, wherein the control of the plurality of animal tests comprises transmitting commands to a corresponding plurality of child controller boards, each controller board in electrical communication with a corresponding main controller of the plurality of main controllers, each child controller board comprising one of a reward dispenser controller board, an environmental controller board, a tone controller board, and a noise controller board.
67. A method of controlling an environment of an animal testing enclosure, comprising:

generating electrical signals over a modular interface connector, the electrical signals defining a command for a controller board, the controller board configured to receive input from or generate output to the environment within the animal testing enclosure in response to receiving the command, the controller board comprising one or more of a tone controller board, environmental controller board, noise controller board, or reward dispenser board; and

receiving electrical signals over the modular interface connector from the controller board indicating a status of execution of the command by the controller board.
68. The method of embodiment 67, further comprising:

sensing presence or absence of the controller board by generating electrical signals over a second modular interface connector, the second electrical signals defining a command, and sensing whether a response to the command is received over the second modular interface connector; and

adapting an animal testing capability based on the sensing.
69. A system for animal testing, comprising:

a plurality of animal enclosures, an environment of each animal enclosure electronically controlled by a plurality of child controller boards; and

a plurality of main controllers, each main controller in network communication with a corresponding one of the plurality of child controller boards for a particular animal enclosure, and configured to control a test using the plurality of child controllers and the corresponding animal enclosure.
70. The system of embodiment 69, wherein the plurality of main controllers executes in a separate execution thread of a common hardware computer.
71. The system of embodiment 70, wherein each separate execution thread is configured to execute a web server, the web server configured to execute the corresponding main controller of the plurality of main controllers for the execution thread.
72. A method of executing an animal test or training protocol using an electronically controlled animal testing enclosure, the electronic control provided by a plurality of child controller circuit boards, comprising:

receiving, via a first computer, input defining a subject name;

requesting, from a second computer, an experiment to administer based on the subject name;

requesting and receiving test control information based on the experiment;

sending instructions implementing a display controller to one of the plurality of child controller boards based on the test control information, wherein the child controller board is configured to execute the instructions;

receiving, by the first computer a connection request from the display controller;

administering, by the first computer, the experiment by sending and receiving data over the connection.
73. The method of embodiment 72, further comprising:

determining, at the first computer, whether there are more experiments to administer for the subject name; and

iteratively performing the method of embodiment 72 based on the determining.
74. A method of executing an animal test or training protocol using an electronically controlled animal enclosure, the electronic control provided by a plurality of child controllers, comprising:

executing, via an electronic hardware computer, a boot script, the boot script configured to: instantiate a central hub and a main controller; and instantiate a web browser, the web browser configured to fetch a uniform reference locator (URL) from the main controller;

downloading, from the main controller, a display controller;

instantiating the display controller within the web browser;

establishing communication between the display controller and the main controller;

initiating an experiment via the established communication; and

storing, via the main controller, results of the experiment.
75. The method of embodiment 74, wherein the main controller is configured to store the results to a global server.
76. The method of embodiment 74, further comprising requesting experiment control information from a meta runner.
77. The method of embodiment 76, wherein the experiment control information is a script that when executed performs the experiment.
78. A system for animal testing, comprising:

a main controller computer, configured to execute a web browser and an execution thread, the execution thread configured to execute a web server, the web server configured to execute a main controller and a central hub, and

the main controller configured to execute a boot script, the boot script configured to instantiate the main controller, the central hub, and the web browser.
79. The system of embodiment 78, wherein the boot script is further configured to cause the web browser to download a display controller from the main controller, wherein the web browser is configured to instantiate the display controller.
80. The system of embodiment 78, further comprising a plurality of child controllers, wherein the main controller is configured to communicate with each of the plurality of child controllers.
81. The system of embodiment 80, wherein the plurality of child controllers comprise the display controller, an environmental controller, and a noise controller.
82. A device for the controlled dispensing of rewards comprising:

a base;

a mount securing the base to a reward container at an angle with respect to the reward container;

a pathway coupled to the reward container, the pathway having an entry configured to receive a reward and an exit for dispensing the reward;

a gate disposed within the pathway, the gate configured to selectively allow a reward into the pathway; and

a feedback sensor configured to output a signal when the reward enters the pathway.
83. The device of embodiment 82, wherein the reward container includes a dispensing plate coupled to a motor configured to incrementally rotate the dispensing plate with respect to the reward container.
84. The device of embodiment 82, wherein the angle is about ten degrees with respect to the reward container.
85. The device of embodiment 82, wherein the sensor is an infrared sensor.
86. The device of embodiment 82, wherein the infrared sensor is positioned at or adjacent to the exit for dispensing the reward.
87. A system for cognitive testing of non-human animals, comprising:

a plurality of testing systems, each system comprising an animal test enclosure and one or more child controller boards, each child controller board configured to control one or more aspects of an environment of the animal test enclosure;

an experimental launcher computer comprising a hardware processor and a hardware memory, the hardware memory storing instructions that configure the hardware processor to communicate with the one or more child controller boards across the plurality of testing systems to:

start execution of sequences of testing commands using the one or more child controller boards,

display status of the sequences on an electronic display, and

receive results of the sequences of testing commands from the one or more child controller boards and store the results to a stable storage.
88. The system of embodiment 87, wherein the hardware memory stores further instructions that configure the hardware processor to receive one or more attributes of a test subject within one of the animal test enclosures and to store the one or more attributes to a data store.
89. The system of embodiment 88, wherein the attributes include one or more of a subject name, a subject identifier number, a subject weight, a subject sex, or a subject species.
90. The system of embodiment 87, wherein the hardware memory stores further instructions that configure the hardware processor to store the results of the sequences as test log files uploaded from a system for cognitive testing.
91. The system of embodiment 87, wherein the hardware memory stores further instructions that configure the hardware processor to parse the results of the sequences into test result values and display the test result values on an electronic display.
92. The system of embodiment 87, wherein the hardware memory stores further instructions that configure the hardware processor to analyze the results of the sequences as a set.
93. An animal testing system for a non-human primate, comprising:

a floor;

one or more walls coupled to the floor;

a top portion supported by the one or more walls, wherein the one or more walls, the floor, and the top portion define a testing chamber;

an opening, the opening being sized and shaped to allow the non-human primate to enter the testing chamber;

a door being configured to selectively cover the opening;

a grate supported by the one or more walls at a location above the floor;

an interface positioned above the grate and comprising:

a display device disposed so as to be viewable by the non-human primate,

a touch device configured to sense contact by the non-human primate,

a speaker configured to selectively emit an auditory signal,

a pick-up area disposed so as to be accessible by the non-human primate,

a dispense light configured to selectively emit a visual signal, and

a light sensor configured to measure a light level within the testing chamber;

a reward dispenser supported by the top portion and operably connected to the pick-up area;

a main controller circuit board supported by the top portion and comprising a plurality of modular interface connectors; and

a plurality of child controller circuit boards operably connected to the plurality of modular interface connectors.
94. The system of embodiment 93, wherein a first of the plurality of child controller circuit boards is configured to send control signals to the reward dispenser in response to receipt of a command from the main controller circuit board over the corresponding modular interface connector, and send a response to the main controller circuit board based on feedback signals received from the reward dispenser.
95. The system of embodiment 94, wherein a second of the plurality of child controller circuit boards is an environmental controller circuit board, and wherein the environmental controller circuit board is configured to control one or more of house lights and a fan within the testing chamber in response to receipt of a command from the main controller circuit board.
96. The system of embodiment 95, further comprising a temperature sensor configured to measure a temperature of the testing chamber, and wherein the environmental controller circuit board is further configured to receive the temperature measurement from the temperature sensor and adjust the temperature within the enclosure via the fan.
97. The system of embodiment 95, wherein a third of the plurality of child controller circuit boards is a tone controller circuit board, and wherein the tone controller circuit board is configured to control the speaker in response to receipt of a command from the main controller circuit board.
98. The system of embodiment 97, wherein a fourth of the plurality of child controller circuit boards is a noise controller circuit board, and wherein the noise controller circuit board is configured to control a level of white noise within the testing chamber via a speaker and a microphone in response to receipt of a command from the main controller circuit board.
99. The system of embodiment 98, wherein a fifth of the plurality of child controller circuit boards is a display controller circuit board, and wherein the display controller circuit board is configured to control the display device and the touch device.
100. The system of embodiment 99, further comprising a subject camera, wherein a sixth child controller circuit board of the plurality of child controller circuit boards is a video controller circuit board, and wherein the video controller circuit board is configured to control video recording of a subject within the testing chamber via the subject camera.
101. The system of embodiment 93, wherein the main controller circuit board is configured to present a challenge to the non-human primate in the form of a visual stimulus on the display device and receive input indicating a response from the non-human primate from the touch device.
102. The system of embodiment 93, further comprising one or more wheels.
103. The system of embodiment 93, wherein the grate is positioned horizontally within the testing chamber at a distance less than 12 inches from the floor.
104. The system of embodiment 93, further comprising one or more reinforcement braces attaching the top portion to at least one of the walls.
105. A system for cognitive testing, the system comprising:

an output device configured to change state during a cognitive test;

an instruction receiver configured to receive a test instruction including an instruction type and an instruction parameter; and

an interpreter in data communication with the instruction receiver and the output device, the interpreter configured to:
generate a control amount indicating a quantity of adjustment to the output device by comparing at least a portion of environmental information for an area in which the cognitive test is performed with the instruction parameter;
generate a control message indicating a change in state of the output device using the test instruction and the control amount; and
transmit the control message to the output device.
106. The system of Embodiment 105, wherein the environmental information for the area includes one or more of: light, sound, vibration, temperature, humidity, or output level of the output device.
107. The system of Embodiment 105, further comprising a feedback device configured to detect the environmental information for the area in which the cognitive test is performed.
108. The system of Embodiment 105, Embodiment 197, or Embodiment 199, wherein the instruction receiver is further configured to:

receive a list of test instructions including the test instruction, the list of test instructions being specified in a domain specific language; and

parse the test instruction from the list of test instructions using the domain specific language, and

wherein the interpreter is configured to identify a format for the control message for the output device using a cognitive testing activity specified in the domain specific language.
109. The system of Embodiment 105, Embodiment 197, or Embodiment 199, wherein the system further comprises:

a data store configured to store a control message format for a cognitive testing activity for controlling the output device,

wherein the interpreter is configured to generate the control message by at least identifying the control message format for the output device using the cognitive testing activity indicated by the test instruction.
110. The system of Embodiment 109, wherein the system further comprises a second output device configured to change state during the cognitive test, and

wherein the data store is further configured to store a second control message format for the cognitive testing activity for controlling the second output device, and

wherein the interpreter is further configured to:
generate a second control amount indicating a second quantity of adjustment to the second output device by comparing at least a portion of the environmental information with the instruction parameter;
generate a second control message indicating a change in state of the second output device using the test instruction and the second control amount, the second control message being different from the control message; and
transmit the second control message to the second output device.
111. The system of Embodiment 105, wherein the interpreter is further configured to:

receive a response to the change in state; and

store the response in a data store.
112. The system of Embodiment 105, Embodiment 197, or Embodiment 199, further comprising a testing coordination server in data communication with a plurality of instruction receivers including the instruction receiver, wherein the testing coordination server transmits and receives messages to at least one of the plurality of instruction receivers associated with a plurality of sequences of testing instructions.
113. The system of Embodiment 112, wherein the testing coordination server receives a study design to determine a sequence of testing instruction to be run for a given test subject.
114. The system of Embodiment 113, wherein the study design is implemented by logic comprising a fixed list of activities.
115. The system of Embodiment 113, wherein the study design is implemented by logic comprising an algorithm that takes into account prior test results to select a test to be run for the given test subject.
116. The system of Embodiment 113, wherein the study design is implemented by logic expressed in a domain specific language to select a test to be run for the given test subject.
117. The system of Embodiment 116, wherein the test to be run is selected to further the understanding of a cognitive response of the test subject to given conditions or treatments.
118. The system of Embodiment 116, wherein the test to be run includes an assay to perform at least one of the following: determine a cognitive profile of the given test subject, diagnose or identify a change in cognitive function brought about by heredity, disease, injury, or age, or monitor or measure a response of the given test subject to therapy, or for drug screening.
119. The system of Embodiment 116, wherein the test to be run includes an assay to diagnose or identify a change in cognitive function brought about by heredity, disease, injury, or age.
120. The system of Embodiment 116, wherein the test to be run includes an assay to monitor or measure a response of the given test subject to therapy.
121. The system of Embodiment 120, wherein the given test subject is undergoing rehabilitation.
122. The system of Embodiment 116, wherein the test to be run includes an assay for drug screening.
123. The system of Embodiment 122, wherein the drug screening is for identifying agents that can enhance long term memory.
124. The system of Embodiment 116, wherein the test to be run includes a training protocol including instructions that configure the output device to one or more states to improve a cognitive function of the test subject either alone or in conjunction with a pharmaceutical treatment.
125. The system of Embodiment 124, wherein the training protocol comprises at least one of: cognitive training, motor training, instructions to configure the output device to present process-specific tasks, instructions to configure the output device to present skill-based tasks, or an augmented cognitive training protocol.
126. The system of Embodiment 124, wherein the training protocol comprises motor training.
127. The system of Embodiment 124, wherein the training protocol comprises instructions to configure the output device to present process-specific tasks.
128. The system of Embodiment 124, wherein the training protocol comprises instructions to configure the output device to present skill-based tasks.
129. The system of Embodiment 124, wherein the training protocol is an augmented cognitive training protocol.
130. The system of Embodiment 129, wherein the augmented cognitive training protocol includes instructions to configure the output device for rehabilitating a cognitive or motor deficit.
131. The system of Embodiment 130, wherein the cognitive or motor deficit is associated with a neurotrauma disorder.
132. The system of Embodiment 131, wherein the neurotrauma disorder comprises at least one of a stroke, a traumatic brain injury (TBI), a head trauma, and a head injury.
133. The system of Embodiment 129, wherein the augmented cognitive training protocol includes instructions to configure the output device to enhance a cognitive or motor function.
134. The system of Embodiment 105, wherein the output device is configured to adjust an environmental condition in the area in which the cognitive test is performed, and wherein the environmental condition is at least one of light, sound, temperature, or humidity.
135. The system of Embodiment 134, wherein the environmental condition is at least one of light, sound, temperature, or humidity.
136. The system of Embodiment 134, wherein the test instruction includes a set-point for the environmental condition, and wherein the interpreter is configured to periodically receive a detected value for the environmental condition; and generate a second command message for the output device, the second command message including information to adjust the environmental condition to the set-point, the information to adjust the environmental condition determined based on a comparison of the set-point and the detected value.
137. The system of Embodiment 105, Embodiment 197, or Embodiment 199, wherein the output device controls presentation of a sensory stimulus.
138. The system of Embodiment 137, wherein the sensory stimulus comprises one of a visual stimulus, an auditory stimulus, a mechanical stimulus, an olfactory stimulus, or a taste stimulus.
139. The system of Embodiment 105, Embodiment 197, or Embodiment 199, wherein the output device is further configured to transmit a message to the interpreter.
140. The system of Embodiment 139, wherein the message includes an acknowledgement, a notification event, an environmental measurement, status confirmation, or warning.
141. The system of Embodiment 139, wherein the interpreter comprises an interrupt-driven state machine configured to handle the message after receipt by the interpreter, the interrupt-driven state machine implements a cognitive test protocol.
142. The system of Embodiment 141, wherein the interrupt-driven state machine is configured to identify a subsequent test instruction to execute based on the message.
143. The system of Embodiment 141, wherein the cognitive test protocol is expressed in a domain specific language defining a sequence of testing commands in terms of stimuli sets, intervals, response event tests, and system events.
144. The system of Embodiment 143, wherein the domain specific language may include instructions to cause lookup of previous test results, and wherein generating the control message is further based on the previous test results, wherein at least one of difficulty or duration for a test are adjusted based on the previous test results.
145. The system of Embodiment 105, Embodiment 197, or Embodiment 199, wherein the cognitive test measures a cognitive or motor function in a subject.
146. The system of Embodiment 105, Embodiment 197, or Embodiment 199, wherein the cognitive test measures a change in a cognitive or motor function in a subject brought about by heredity, disease, injury, or age.
147. The system of Embodiment 105, Embodiment 197, or Embodiment 199, wherein the cognitive test measures a change in a cognitive or motor function in a subject undergoing therapy or treatment of a neurological disorder.
148. The system of Embodiment 147, wherein the cognitive test includes instructions to configure the output hardware to present a training protocol.
149. The system of Embodiment 148, wherein the training protocol comprises cognitive training.
150. The system of Embodiment 148, wherein the training protocol comprises motor training.
151. The system of Embodiment 148, wherein the training protocol comprises process-specific tasks.
152. The system of Embodiment 148, wherein the training protocol comprises skill-based tasks.
153. The system of Embodiment 148, wherein the training protocol includes instructions to configure the output device to one or more states that enhances a cognitive or motor function.
154. The system of Embodiment 148, wherein the training protocol includes instructions to configure the output device to one or more states that rehabilitates a cognitive deficit or a motor deficit associated with a neurological disorder.
155. The system of Embodiment 154, wherein the cognitive deficit comprises a deficit in memory formation.
156. The system of Embodiment 155, wherein the deficit in memory formation comprises a deficit in long-term memory formation.
157. The system of Embodiment 154, wherein the neurological disorder comprises a neurotrauma.
158. The system of Embodiment 157, wherein the neurotrauma comprises stroke or traumatic brain injury.
159. The system of any one of Embodiments 148-158, further comprising screening for drugs that increase the efficiency of the training protocol.
160. The system of any one of Embodiments 148-158, wherein the training protocol is an augmented training protocol and further comprises administering an augmenting agent in conjunction with training.
161. The system of Embodiment 105, Embodiment 197, or Embodiment 199, wherein the output device comprises a reward dispenser and wherein the test instruction identifies a quantity of a reward to dispense.
162. The system of Embodiment 161, wherein the reward comprises an edible reward.
163. The system of Embodiment 162, wherein the edible reward comprises a pellet, liquid, candy, tablet, food item, or paste.
164. A method of cognitive testing, the method comprising:

receiving a cognitive test configuration indicating a testing unit for performing a cognitive test;

generating a command to adjust a testing hardware element included in the testing unit using the cognitive test configuration and calibration information indicating a state of the testing hardware element; and

transmitting the command to the testing unit.
165. The method of Embodiment 164, wherein the calibration information includes one of: light, sound, vibration, temperature, humidity, or output level of the testing hardware element or the testing unit.
166. The method of Embodiment 164, further comprising receiving the calibration information from the testing unit indicating the state of a testing hardware element included in the testing unit.
167. The method of Embodiment 164, wherein the cognitive test configuration is specified in a domain specific language, and wherein the method further comprises identifying a format for the command for the testing hardware element using a cognitive testing activity specified in the domain specific language.
168. The method of Embodiment 164, further comprising:

storing the calibration information for the testing hardware element in a data store;

determining the calibration information deviates from a calibration threshold for the testing hardware element; and

generating an alert for the testing hardware element, the alert indicating a possible malfunction for the testing hardware element.
169. The method of Embodiment 168, further comprising generating a termination command to end the cognitive test, the termination command including an indication of the possible malfunction for the testing hardware element.
170. The method of Embodiment 164, further comprising:

identifying a resource included in the cognitive test configuration;

determining the resource is not accessible by the testing unit; and

transferring the resource to a location accessible by the testing unit.
171. The method of Embodiment 164, further comprising:

receiving a response to the command; and

storing the response in a data store.
172. An apparatus for cognitive testing, the apparatus comprising:

means for receiving a cognitive test configuration indicating a testing unit for performing a cognitive test;

means for generating a command to adjust a testing hardware element included in the testing unit using the cognitive test configuration and calibration information indicating a state of the testing hardware element; and

means for transmitting the command to the testing unit.
173. The apparatus of Embodiment 172, wherein the calibration information includes one of: light, sound, vibration, temperature, humidity, or output level of the testing hardware element or the testing unit.
174. The apparatus of Embodiment 172, further comprising means for receiving the calibration information from the testing unit indicating the state of a testing hardware element included in the testing unit.
175. The apparatus of Embodiment 172, wherein the means for receiving the cognitive test configuration receives cognitive test configuration specified in a domain specific language, and wherein the apparatus further comprises means for identifying a format for the command for the testing hardware element using a cognitive testing activity specified in the domain specific language.
176. The apparatus of Embodiment 172, further comprising:

means for storing the calibration information for the testing hardware element in a data store;

means for determining the calibration information deviates from a calibration threshold for the testing hardware element; and

means for generating an alert for the testing hardware element, the alert indicating a possible malfunction for the testing hardware element.
177. The apparatus of Embodiment 176, further comprising means for generating a termination command to end the cognitive test, the termination command including an indication of the possible malfunction for the testing hardware element.
178. The apparatus of Embodiment 172, further comprising:

means for identifying a resource included in the cognitive test configuration;

means for determining the resource is not accessible by the testing unit; and

means for transferring the resource to a location accessible by the testing unit.
179. The apparatus of Embodiment 172, further comprising:

means for receiving a response to the command; and

means for storing the response in a data store.
180. A system for cognitive testing of an animal, comprising:

means for providing a sequence of testing commands to a cognitive testing apparatus;

means for receiving a response from the cognitive testing apparatus, the response associated with at least one testing command included in the sequence of testing commands; and

means for adjusting the cognitive testing apparatus to provide feedback regarding the response.
181. The system of Embodiment 180, wherein the means for providing a sequence of testing commands comprises a central hub, and wherein the means for receiving a response and adjusting the cognitive testing apparatus comprises a main controller and one or more independent child controllers.
182. The system of Embodiment 181, wherein the means for providing the sequence of testing commands further comprises a meta hub configured to communicate, via a network, with a plurality of central hubs, the plurality of central hubs including the central hub.
183. The system of Embodiment 182, wherein the meta hub resides on an internet server and the central hub, the main controller, and independent child controllers run on software downloaded to the cognitive testing apparatus at a predetermined secure testing facility.
184. The system of Embodiment 182, wherein the meta hub resides on an internet server and the central hub, the main controller, and independent child controllers run on software downloaded to the cognitive testing apparatus of a subject under test.
185. The system of Embodiment 184, wherein the cognitive testing apparatus comprises a portable electronic communication device.
186. The system of Embodiment 185, wherein the portable electronic communication device comprises a smartphone or a table computer.
187. The system of Embodiment 182, wherein the meta hub executes in a first thread on an internet server, and wherein the central hub and the main controller executed in a second thread on the internet server, and a virtual display controller provides output to a web browser on a remote computer in data communication with the internet server.
188. The system of Embodiment 187, wherein the internet server is a server cluster configured to share load for at least 10,000 threads to execute corresponding instances of the central hub and the main controller.
189. The system of Embodiment 180, wherein the animal is a non-human animal.
190. The system of Embodiment 189, wherein the non-human animal is a non-human primate.
191. The system of Embodiment 190, wherein the non-human primate is a macaque.
192. The system of Embodiment 189, wherein the non-human animal is a non-human mammal.
193. The system of Embodiment 192, wherein the non-human mammal is one of a dog, a mouse, or a rat.
194. The system of Embodiment 189, wherein the non-human animal is a vertebrate.
195. The system of Embodiment 189, wherein the non-human animal is an invertebrate.
196. The system of Embodiment 195, wherein the invertebrate is a fruit fly.
197. A system for cognitive testing, the system comprising:

an output device configured to indicate or change state during a cognitive test;

an instruction receiver configured to receive a test instruction including an instruction type and an instruction parameter; and

an interpreter in data communication with the instruction receiver and the output device, the interpreter configured to generate a control message about a state of the output device.
198. The system of Embodiment 197, or Embodiment 199, wherein the interpreter is further configured to:

generate a control amount indicating a quantity of adjustment to the output device by comparing at least a portion of environmental information for an area in which the cognitive test is performed with the instruction parameter;

generate a control message indicating a change in state of the output device using the test instruction and the control amount; and

transmit the control message to the output device.
199. A system for cognitive testing, the system comprising:

an output device configured to change state during a cognitive test;

an instruction receiver configured to receive a test instruction including an instruction type and an instruction parameter; and

an interpreter in data communication with the instruction receiver and the output device, the interpreter configured to adjust a specific test execution using feedback from at least one device included in the system.
200. The system of Embodiment 197 or Embodiment 199, wherein the interpreter is further configured to:

receive a response to a change in state for the output device; and

store the response in a data store.
201. The system of Embodiment 197 or Embodiment 199, wherein the output device is configured to adjust an environmental condition in an area in which the cognitive test is performed.
202. The system of Embodiment 201, wherein the environmental condition is at least one of light, sound, temperature, or humidity.
203. The system of Embodiment 201, wherein the test instruction includes a set-point for the environmental condition, and wherein the interpreter is configured to periodically receive a detected value for the environmental condition; and generate a second command message for the output device, the second command message including information to adjust the environmental condition to the set-point, the information to adjust the environmental condition determined based on a comparison of the set-point and the detected value.
204. A system, comprising:

a plurality of cognitive testing systems; and

a meta hub processor being in data communication with the plurality of cognitive testing systems and configured to automatically coordinate information regarding multiple test subjects and multiple sequences of testing commands among the plurality of cognitive testing systems;

wherein each cognitive testing system comprises:
a central hub processor configured to provide a testing command for a testing station that is configured to accommodate one of a plurality of the animals;
a plurality of secondary controllers configured to control the testing station, wherein the testing command is associated with one of the plurality of secondary controllers; and
a main controller configured to i) receive the testing command from the central hub
processor, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers,

wherein the one of the plurality of secondary controllers is configured to control the testing station based at least in part on the operating parameter.
205. The system of Embodiment 204, wherein each cognitive testing system further comprises a printed circuit board, wherein the main controller is supported by the printed circuit board, wherein the plurality of secondary controllers comprise a logical secondary controller positioned within the printed circuit board and a physical secondary controller positioned outside and electrically connected to the printed circuit board.
206. The system of Embodiment 205, wherein the logical secondary controller comprises at least one of the following:

a display controller configured to control data interface between the animal and the testing station; and

a video controller configured to control video streams to and from the testing station.
207. The system of Embodiment 205, wherein the physical secondary controller comprises at least

one of the following:

a tone controller configured to control a success or failure tone for the cognitive testing;

a noise controller configured to control noise levels in the testing station;

a reward dispensing controller configured to control reward dispensing in the testing station; and

an environmental controller configured to control a testing environment of the testing station.
208. The system of any of Embodiment 205-207, wherein the operating parameter is configured to control the logical and physical secondary controllers to perform their respective control operations on the testing station.
209. The system of Embodiment 204, wherein the main controller and the secondary controller are located in the testing station.
210. The system of Embodiment 204, further comprising:

a central hub simulator configured to simulate an operation of the central hub processor; and

a main controller simulator configured to simulate an operation of the main controller.
211. The system of Embodiment 204, wherein the one of the plurality of secondary controllers is configured to control at least one hardware component of the testing station or at least one environmental condition in the testing station based at least in part on the operating parameter.
212. The system of Embodiment 211, wherein the at least one hardware component comprises an input device, an output device, a data processing device and a reward dispensing device of the testing station.
213. The system of Embodiment 211, wherein the at least one environmental condition comprises temperature, humidity, light, or sound in the testing station.
214. The system of Embodiment 204, wherein the testing command comprises computer-readable instructions associated with the one of the plurality of secondary controllers.
215. The system of Embodiment 214, wherein the main controller is configured to determine the one of the plurality of secondary controllers based on the computer-readable instructions, and generate the operating parameter for the one of the plurality of secondary controllers to control at least one hardware component of the testing station and/or at least one environmental condition in the testing station.
216. The system of Embodiment 204, wherein the animal is a non-human primate.
217. The system of Embodiment 204, wherein the animal is a human.
218. A system for cognitive testing of an animal, comprising:

a main controller configured to receive a testing command from a central hub processor,

wherein the testing command is associated with one of a plurality of secondary controllers configured to control a testing station that accommodates the animal,

wherein the main controller is further configured to i) determine the one of the plurality of secondary controllers associated with the received testing command, ii) generate an operating parameter for the one of the plurality of secondary controllers based at least in part on the received testing command and iii) provide the generated operating parameter to the one of the plurality of secondary controllers.
219. The system of Embodiment 218, wherein the main controller comprises:

a first interface circuit configured to interface data communication between the central hub processor and the main controller;

a second interface circuit configured to interface data communication between the main controller and the secondary controller; and

a processor configured to determine the one of the plurality of secondary controllers associated with the received testing command and generate the operating parameter for the one of the plurality of secondary controllers based at least in part on the received testing command.
220. The system of Embodiment 219, further comprising a memory storing information indicative of commands received from the central hub processor and associated with the plurality of secondary controllers, wherein the processor is configured to determine the one of the plurality of secondary controllers based at least in part on the information stored in the memory.
221. The system of Embodiment 219, wherein the second interface circuit comprises a plurality of serial ports to be connected to the plurality of secondary controllers, and wherein the processor is configured to detect the one of the plurality of secondary controllers by scanning the serial ports.
222. The system of Embodiment 218, further comprising a printed circuit board, wherein the main controller is supported by the printed circuit board, wherein the at least one secondary controller comprises a logical secondary controller positioned within the printed circuit board and a physical secondary controller positioned outside and electrically connected to the printed circuit board.
223. A system for cognitive testing of an animal, comprising:

a plurality of secondary controllers configured to control a testing station that is configured to accommodate the animal; and

a main controller configured to i) receive a testing command from a central hub

processor, wherein the testing command is associated with one of the plurality of secondary controllers, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers,

wherein the one of the plurality of secondary controllers is configured to control the testing station based at least in part on the operating parameter.
224. The system of Embodiment 223, further comprising a printed circuit board, wherein the main controller is supported by the printed circuit board, wherein the plurality of secondary controllers comprise a logical secondary controller positioned within the printed circuit board and a physical secondary controller positioned outside and electrically connected to the printed circuit board.
225. The system of Embodiment 224, wherein the logical secondary controller comprises at least one of the following:

a display controller configured to control data interface between the animal and the testing station, and

a video controller configured to control video streams to and from the testing station;

and wherein the physical secondary controller comprises at least one of the following:
a tone controller configured to control a success or failure tone for the cognitive testing,
a noise controller configured to control noise levels in the testing station,
a reward dispensing controller configured to control reward dispensing in the testing station; and
an environmental controller configured to control a testing environment of the testing station.
226. A method of cognitive testing of an animal, comprising:

providing a plurality of secondary controllers configured to control a testing station that accommodates the animal;

receiving a testing command from a central hub processor, wherein the testing command is associated with one of the plurality of secondary controllers;

determining the one of the plurality of secondary controllers associated with the received testing command;

generating an operating parameter for the one of the plurality of secondary controllers based at least in part on the received testing command; and

providing the generated operating parameter to the one of the plurality of secondary controllers.
227. The method of Embodiment 226, wherein the determining comprises:

determining whether the received testing command relates to a logical secondary controller function or a physical secondary controller function; and

determining a corresponding physical controller when the received testing command relates to the physical secondary controller function.
228. The method of Embodiment 24, further comprising:

second determining, when the received testing command relates to the logical secondary controller function, whether the received testing command relates to a display controller function or a video controller function;

recognizing a display controller as the one of the plurality of secondary controllers when the received testing command relates to the display controller function; and

recognizing a video controller as the one of the plurality of secondary controllers when the received testing command relates to the video controller function.
229. The method of Embodiment 226, wherein the cognitive testing is used to measure a cognitive or motor function of the animal.
230. The method of Embodiment 226, wherein the cognitive testing is used to measure a change in a cognitive or motor function of the animal brought about by heredity, disease, injury, or age.
231. The method of Embodiment 226, wherein the cognitive testing is used to measure a change in a cognitive or motor function of the animal undergoing therapy or treatment of a neurological disorder.
232. The method of Embodiment 226, wherein the cognitive testing includes a training protocol.
233. The method of Embodiment 232, wherein the training protocol comprises cognitive training.
234. The method of Embodiment 232, wherein the training protocol comprises at least one of: cognitive training, motor training, process-specific tasks, processes for enhancing a cognitive or motor function of the animal, or processes for rehabilitating a cognitive or motor deficit associated with a neurological disorder.
235. The method of Embodiment 232, wherein the training protocol comprises process-specific tasks.
236. The method of Embodiment 232, wherein the training protocol comprises skill-based tasks.
237. The method of Embodiment 232, wherein the training protocol is for use in enhancing a cognitive or motor function of the animal.
238. The method of Embodiment 232, wherein the training protocol is for use in rehabilitating a cognitive or motor deficit associated with a neurological disorder.
239. The method of Embodiment 238, wherein the cognitive deficit is a deficit in memory formation.
240. The method of Embodiment 239, wherein the deficit in memory formation is a deficit in long-term memory formation.
241. The method of Embodiment 238, wherein the neurological disorder is a neurotrauma.
242. The method of Embodiment 241, wherein the neurotrauma is stroke or traumatic brain injury.
243. The method of Embodiment 232, further comprising screening for drugs that increase the efficiency of the training protocol.
244. The method of Embodiment 232, wherein the training protocol is an augmented training protocol and wherein the method further comprises administering an augmenting agent in conjunction with training.
245. A system for cognitive testing of an animal, comprising:

means for receiving a testing command from a central hub processor, wherein the

testing command is associated with one of a plurality of secondary controllers configured to control a testing station that accommodates the animal;

means for determining the one of the plurality of secondary controllers

associated with the received testing command;

means for generating an operating parameter for the one of the plurality of secondary controllers based at least in part on the received testing command; and

means for providing the generated operating parameter to the one of the plurality of secondary controllers.
246. One or more processor-readable storage devices having processor-readable code embodied on the processor-readable storage devices, the processor-readable code for programming one or more processors to perform a method of cognitive testing of an animal, the method comprising:

providing a plurality of secondary controllers configured to control a testing station that accommodates the animal;

receiving a testing command from a central hub processor, wherein the testing command is associated with one of the plurality of secondary controllers;

determining the one of the plurality of secondary controllers associated with the received testing command;

generating an operating parameter for the one of the plurality of secondary controllers based at least in part on the received testing command; and

providing the generated operating parameter to the one of the plurality of secondary controllers.
247. A system for cognitive testing of an animal, comprising:

a central hub processor being in data communication with at least one of a main

controller and a plurality of secondary controllers configured to control a testing station that accommodates the animal, wherein the central hub processor is configured to send a testing command to the main controller, wherein the testing command is associated with one of the plurality of secondary controllers and configured to control the main controller to i) determine the one of the plurality of secondary controllers associated with the testing command, ii) generate an operating parameter for the one of the plurality of secondary controllers based at least in part on the testing command and iii) provide the generated operating parameter to the one of the plurality of secondary controllers.
248. The system of Embodiment 247, wherein the testing command comprises computer-readable instructions associated with the one of the plurality of secondary controllers, and wherein the central hub processor is configured to control the main controller to determine the one of the plurality of secondary controllers based on the computer-readable instructions, and generate the operating parameter for the one of the plurality of secondary controllers to control at least one hardware component of the testing station and/or at least one environmental condition in the testing station.
249. A system for cognitive testing of a non-human animal subject, comprising:

a central hub processor configured to provide a sequence of testing commands;

a main controller configured to receive the testing commands from the central hub processor and parse the received testing commands; and

one or more independent child controllers configured to execute the testing commands, receive responses to the testing commands from the non-human animal subject, and provide feedback regarding the responses.
250. The system of Embodiment 249, wherein the central hub processor is located on a separate computer and configured to communicate data with the main controller over a network.
251. The system of Embodiment 249, wherein the central hub processor and the main controller are located on the same computer.
252. The system of Embodiment 249, wherein the one or more independent child controllers include a physical child controller.
253. The system of Embodiment 252, wherein the physical child controller comprises an Arduino microcontroller.
254. The system of Embodiment 249, wherein the one or more independent child controllers include a virtual child controller.
255. The system of Embodiment 254, wherein the virtual child controller is located on the main controller.
256. The system of Embodiment 254, wherein the virtual child controller is located on a web browser.
257. The system of Embodiment 256, wherein the web browser is located on the main controller.
258. The system of Embodiment 256, wherein the web browser is located on a separate computer and configured to communicate data with the main controller over a network.
259. A computer network for cognitive testing of animal subjects, comprising:

a plurality of cognitive testing systems, wherein each cognitive testing system

comprises a main controller and a plurality of secondary controllers configured to control a testing station that accommodates a non-human animal subject, wherein the main controller is configured to i) receive a testing command, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers, and wherein the one of the plurality of secondary controllers is configured to control the testing station based at least in part on the operating parameter; and

a meta hub processor being in data communication with the plurality of cognitive testing systems and configured to automatically coordinate information regarding multiple test subjects and multiple sequences of testing commands among the plurality of cognitive testing systems.
260. A system for cognitive testing of a human subject, comprising:

a central hub processor configured to provide a sequence of testing commands;

a main controller configured to receive the testing commands from the central hub processor and parse the received testing commands; and

one or more independent child controllers configured to execute the testing commands, receive responses to the testing commands from the human subject, and provide feedback regarding the responses.
261. The system of Embodiment 260, wherein the central hub processor is located on a separate computer and configured to communicate data with the main controller over a network.
262. The system of Embodiment 260, wherein the central hub processor and the main controller are located on the same computer.
263. The system of Embodiment 260, wherein the one or more independent child controllers include a physical child controller.
264. The system of Embodiment 263, wherein the physical child controller comprises an Arduino microcontroller.
265. The system of Embodiment 260, wherein the one or more independent child controllers include a virtual child controller.
266. The system of Embodiment 265, wherein the virtual child controller is located on the main controller.
267. The system of Embodiment 265, wherein the virtual child controller is located on a web browser.
268. The system of Embodiment 267, wherein the web browser is located on the main controller.
269. The system of Embodiment 267, wherein the web browser is located on a separate computer and configured to communicate data with the main controller over a network.
270. A network for cognitive testing of human subjects, comprising:

a plurality of cognitive testing systems, wherein each cognitive testing system comprises a main controller and a plurality of secondary controllers configured to control a testing station that accommodates a human subject, wherein the main controller is configured to i) receive a testing command, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers, and wherein the one of the plurality of secondary controllers is configured to control the testing station based at least in part on the operating parameter; and

a meta hub processor being in data communication with the plurality of cognitive testing systems and configured to automatically coordinate information regarding multiple test subjects and multiple sequences of testing commands among the plurality of cognitive testing systems.
271. A system for cognitive testing of an animal, comprising:

means for providing a sequence of testing commands to the animal;

means for parsing the testing commands to different controllers;

means for receiving a response to the sequence of testing commands from the animal; and

means for providing feedback regarding the response to the animal.
272. The system of Embodiment 271, wherein the means for providing a sequence of testing commands comprises a central hub processor, wherein the means for parsing comprises a main controller, and wherein the means for receiving a response and the means for providing feedback comprise one or more independent child controllers.
273. The system of Embodiment 245, wherein:

the meta hub processor provides test scripts to each central hub processor for execution,

the test scripts, when executed by the respective central hub processors, cause the central hub processors to send commands to corresponding one more of the child controllers and to receive results back such one or more of the child controllers;

the central hub processors log tests results and sends test result logs to the meta hub processor to be saved.
274. An apparatus comprising:

means for separately enclosing each of a plurality of animals;

means for stimulating the animals;

means for monitoring actions of the animals in response to the means for stimulating;

electronic means to support the means for stimulating and the means for monitoring comprising:

a first and a second interface connector to a first bus, wherein the first and second interfaces are each configured to electrically and physically connect to a child controller circuit board, wherein the child controller circuit board is configured to receive input from or generate output to the testing chamber, and comprises each of a reward dispenser controller board, an environmental controller board, a tone controller board, a noise controller board, and a display controller board, each board comprising a hardware board processor, and

a hardware processor configured to communicate with one or more connected hardware board processors over the first bus.

All publications, patents, and patent applications mentioned in this specification are incorporated herein by reference to the same extent as if each individual publication, patent or patent application was specifically and individually incorporated by references

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A system, comprising:
a plurality of cognitive testing systems; and
a meta hub processor being in data communication with the plurality of cognitive testing systems and configured to automatically coordinate information regarding multiple test subjects and multiple sequences of testing commands among the plurality of cognitive testing systems;
wherein each cognitive testing system comprises:
a central hub processor configured to provide a testing command for a testing station that is configured to accommodate one of a plurality of animals;
a plurality of secondary controllers configured to control the testing station, wherein the testing command is associated with one of the plurality of secondary controllers; and
a main controller configured to i) receive the testing command from the central hub processor, ii) determine the one of the plurality of secondary controllers associated with the received testing command, iii) generate an operating parameter for the one of the plurality of secondary controllers based at least in part on the received testing command and iv) provide the generated operating parameter to the one of the plurality of secondary controllers,
wherein the one of the plurality of secondary controllers is configured to control the testing station based at least in part on the operating parameter.

2. The system of claim 1, wherein each cognitive testing system further comprises a printed circuit board, wherein the main controller is supported by the printed circuit board, wherein the plurality of secondary controllers comprise a logical secondary controller positioned within the printed circuit board; and a physical secondary controller positioned outside and electrically connected to the printed circuit board.

3. The system of claim 2, wherein the logical secondary controller comprises at least one of the following:
a display controller configured to control data interface between the animal and the testing station; and
a video controller configured to control video streams to and from the testing station.

4. The system of claim 2, wherein the physical secondary controller comprises at least one of the following:
a tone controller configured to control a success or failure tone for the cognitive testing;
a noise controller configured to control noise levels in the testing station;
a reward dispensing controller configured to control reward dispensing in the testing station; and
an environmental controller configured to control a testing environment of the testing station.

5. The system of claim 2, wherein the operating parameter is configured to control the logical and physical secondary controllers to perform their respective control operations on the testing station.

6. The system of claim 1, further comprising:
a central hub simulator configured to simulate an operation of the central hub processor; and
a main controller simulator configured to simulate an operation of the main controller.

7. The system of claim 1, wherein the one of the plurality of secondary controllers is configured to control at least one hardware component of the testing station or at least one environmental condition in the testing station based at least in part on the operating parameter.

8. The system of claim 7, wherein the at least one hardware component comprises an input device, an output device, a data processing device, and a reward dispensing device of the testing station.

9. The system of claim 1, wherein the testing command comprises computer-readable instructions associated with the one of the plurality of secondary controllers.

10. The system of claim 1, wherein the main controller is configured to determine the one of the plurality of secondary controllers based on the computer-readable instructions, and generate the operating parameter for the one of the plurality of secondary controllers to control at least one hardware component of the testing station and/or at least one environmental condition in the testing station.

11. The system of claim 1, wherein:
the meta hub processor provides test scripts to each central hub processor for execution,
the test scripts, when executed by the respective central hub processors, cause the central hub processors to send commands to corresponding one more of the child controllers and to receive results back such one or more of the child controllers;
the central hub processors log tests results and sends test result logs to the meta hub processor to be saved.

12. A system for cognitive testing of an animal, comprising:
a main controller configured to receive a testing command from a central hub processor, wherein the testing command is associated with one of a plurality of secondary controllers configured to control a testing station that accommodates the animal,
wherein the main controller is further configured to:
determine the one of the plurality of secondary controllers associated with the received testing command,
generate an operating parameter for the one of the plurality of secondary controllers based at least in part on the received testing command and
provide the generated operating parameter to the one of the plurality of secondary controllers.

13. A method of cognitive testing of an animal, comprising:
providing a plurality of secondary controllers configured to control a testing station that accommodates the animal;
receiving a testing command from a central hub processor, wherein the testing command is associated with one of the plurality of secondary controllers;
determining the one of the plurality of secondary controllers associated with the received testing command;
generating an operating parameter for the one of the plurality of secondary controllers based at least in part on the received testing command; and
providing the generated operating parameter to the one of the plurality of secondary controllers.

14. The method of claim 13, wherein the determining comprises:
determining whether the received testing command relates to a logical secondary controller function or a physical secondary controller function; and
determining a corresponding physical controller when the received testing command relates to the physical secondary controller function.

15. The method of claim 13, further comprising:
second determining, when the received testing command relates to the logical secondary controller function, whether the received testing command relates to a display controller function or a video controller function;
recognizing a display controller as the one of the plurality of secondary controllers when the received testing command relates to the display controller function; and
recognizing a video controller as the one of the plurality of secondary controllers when the received testing command relates to the video controller function.

16. The method of claim 13, wherein the cognitive testing is used to measure a cognitive or motor function of the animal.

17. The method of claim 13, wherein the cognitive testing is used to measure a change in a cognitive or motor function of the animal brought about by heredity, disease, injury, or age.

18. The method of claim 13, wherein the cognitive testing is used to measure a change in a cognitive or motor function of the animal undergoing therapy or treatment of a neurological disorder.

19. The method of claim 13, wherein the cognitive testing includes a training protocol.

20. The method of claim 19, wherein the training protocol comprises at least one of: cognitive training, motor training, process-specific tasks, processes for enhancing a cognitive or motor function of the animal, or processes for rehabilitating a cognitive or motor deficit associated with a neurological disorder.
